(19) United States
(12) Patent Application Publication
(10) Pub. No.: US 2006/0115826 A1
(43) Pub. Date: Jun. 1, 2006

(54) Gene expression profiling for identification monitoring and treatment of multiple sclerosis

(76) Inventors: Michael Bevilacqua, Boulder, CO (US); Victor V. Tryon, Woodinville, WA (US); Danute Bankaitis-Davis, Longmont, CO (US); Lisa Siconolfi, Westminster, CO (US); David B. Trollinger, Boulder, CO (US)

Correspondence Address: BROMBERG & SUNSTEIN LLP, 125 SUMMER STREET, BOSTON, MA, 02110-1618, US

(21) Appl. No.: 11/155,930

(22) Filed: Jun. 16, 2005

Related U.S. Application Data
(63) Continuation-in-part of application No. 10/742,458, filed on Dec. 19, 2003.
(63) Continuation-in-part of application No. 10/291,225, filed on Nov. 8, 2002, now Pat. No. 6,960,439.
Application No. 10/291,225 is a continuation-in-part of application No. 09/821,850, filed on Mar. 29, 2001, now Pat. No. 6,692,916.
Application No. 09/821,850 is a continuation-in-part of application No. 09/605,581, filed on Jun. 28, 2000, now abandoned.
(60) Provisional application No. 60/435,257, filed on Dec. 19, 2002.
(60) Provisional application No. 60/141,542, filed on Jun. 28, 1999.
(60) Provisional application No. 60/195,522, filed on Apr. 7, 2000.

(51) Int. Cl. C12Q 1/68; G06F 19/00

(52) U.S. Cl. 435/6; 702/20

(57) ABSTRACT

A method is provided in various embodiments for determining a profile data set for a subject with multiple sclerosis or inflammatory conditions related to multiple sclerosis based on a sample from the subject, wherein the sample provides a source of RNAs. The method includes using amplification for measuring the amount of RNA corresponding to at least 2 constituents from Table 1. The profile data set comprises the measure of each constituent, and amplification is performed under measurement conditions that are substantially repeatable.

### RELATED REFERENCES

The present application is a continuation-in-part of U.S. application Ser. No. 10/742,458, filed Dec. 19, 2003, incorporated by reference herein, which claims priority from provisional patent application Ser. No. 60/435257, filed Dec. 19, 2002, incorporated by reference herein. The present application is also a continuation in part of application Ser. No. 10/291,225, filed Nov. 8, 2002, incorporated by reference herein, which is a continuation in part of application Ser. No. 09/821,850, filed Mar. 29, 2001, incorporated by reference herein, which in turn is a continuation in part of application Ser. No. 09/605,581, filed Jun. 28, 2000, by the same inventors herein, which application claims priority from provisional application Ser. No. 60/141,542, filed Jun. 28, 1999 and provisional application Ser. No. 60/195,522 filed Apr. 7, 2000, both incorporated by reference herein.

### TECHNICAL FIELD AND BACKGROUND ART

The present invention relates to use of gene expression data, and in particular to use of gene expression data in identification, monitoring and treatment of multiple sclerosis and in characterization and evaluation of inflammatory conditions of a subject induced or related to multiple sclerosis.

The prior art has utilized gene expression data to determine the presence or absence of particular markers as diagnostic of a particular condition, and in some circumstances have described the cumulative addition of scores for over expression of particular disease markers to achieve increased accuracy or sensitivity of diagnosis. Information on any condition of a particular patient and a patient's response to types and dosages of therapeutic or nutritional agents has become an important issue in clinical medicine today not only from the aspect of efficiency of medical practice for the health care industry but for improved outcomes and benefits for the patients.

### SUMMARY OF THE INVENTION

In a first embodiment there is provided a method for determining a profile data set for a subject with multiple sclerosis or inflammatory conditions related to multiple sclerosis based on a sample from the subject, the sample providing a source of RNAs, the method comprising using amplification for measuring the amount of RNA corresponding to at least 2 constituents from Table 1 and arriving at a measure of each constituent, wherein the profile data set comprises the measure of each constituent and wherein amplification is performed under measurement conditions that are substantially repeatable.

In addition, the subject may have presumptive signs of multiple sclerosis including at least one of altered sensory, motor, visual or proprioceptive system with at least one of numbness or weakness in one or more limbs, often occurring on one side of the body at a time or the lower half of the body, partial or complete loss of vision, frequently in one eye at a time and often with pain during eye movement, double vision or blurring of vision, tingling or pain in numb areas of the body, electric-shock sensations that occur with certain head movements, tremor, lack of coordination or unsteady gait, fatigue, dizziness, muscle stiffness or spasticity, slurred speech, paralysis, problems with bladder, bowel or sexual function, and mental changes such as forgetfulness or difficulties with concentration, relative to medical standards, or the inflammatory conditions related to multiple sclerosis may be inflammatory.

In other embodiments, the measurement conditions that are substantially repeatable may be within a degree of repeatability of better than five percent, or better than three percent and the efficiencies of amplification for all constituents may be substantially similar wherein the efficiency of amplification for all constituents is within two percent, or alternatively, is less than one percent. In such embodiments, the sample may be selected from the group consisting of blood, a blood fraction, body fluid, a population of cells and tissue from the subject.

In another embodiment there is provided a method of characterizing multiple sclerosis or inflammatory conditions related to multiple sclerosis in a subject, based on a sample from the subject, the sample providing a source of RNAs, the method comprising assessing a profile data set of a plurality of members, each member being a quantitative measure of the amount of a distinct RNA constituent in a panel of constituents selected so that measurement of the constituents enables characterization of the presumptive signs of a systemic infection, wherein such measure for each constituent is obtained under measurement conditions that are substantially repeatable.

In addition, the subject may have presumptive signs of multiple sclerosis including at least one of altered sensory, motor, visual or proprioceptive system with at least one of numbness or weakness in one or more limbs, often occurring on one side of the body at a time or the lower half of the body, partial or complete loss of vision, frequently in one eye at a time and often with pain during eye movement, double vision or blurring of vision, tingling or pain in numb areas of the body, electric-shock sensations that occur with certain head movements, tremor, lack of coordination or unsteady gait, fatigue, dizziness, muscle stiffness or spasticity, slurred speech, paralysis, problems with bladder, bowel or sexual function, and mental changes such as forgetfulness or difficulties with concentration, relative to medical standards, or alternatively, the subject may have presumptive signs of multiple sclerosis that are related to inflammatory conditions. In such embodiments, assessing may further comprises comparing the profile data set to a baseline profile data set for the panel, wherein the baseline profile data set is related to the multiple sclerosis or inflammatory conditions related to multiple sclerosis to be characterized.

In other embodiments, the efficiencies of amplification for all constituents are substantially similar and the multiple sclerosis or inflammatory conditions related to multiple sclerosis are from a microbial infection, more particularly a bacterial infection, or a eukaryotic parasitic infection, or a viral infection, or a fungal infection or are related to systemic inflammatory response syndrome (SIRS). More particularly, the multiple sclerosis or inflammatory conditions that are related to multiple sclerosis may be from bacteremia, viremia, or fungemia, or from septicemia due to any class of microbe. In addition, the multiple sclerosis or inflammatory conditions related to multiple sclerosis may be with respect to a localized tissue of the subject and the sample may be derived from a tissue or fluid of a type distinct from that of the localized tissue.

Other embodiments include storing the profile data set in a digital storage medium, wherein storing the profile data set may include storing it as a record in a database.

Yet another embodiment provides a method for evaluating multiple sclerosis or inflammatory conditions related to multiple sclerosis in a subject based on a first sample from the subject, the sample providing a source of RNAs, the method comprising deriving from the first sample a first profile data set, the profile data set including a plurality of members, each member being a quantitative measure of the amount of a distinct RNA constituent in a panel of constituents selected so that measurement of the constituents enables evaluation of the multiple sclerosis or inflammatory conditions related to multiple sclerosis wherein such measure for each constituent is obtained under measurement conditions that are substantially repeatable. The method also includes producing a calibrated profile data set for the panel, wherein each member of the calibrated profile data set is a function of a corresponding member of the first profile data set and a corresponding member of a baseline profile data set for the panel, and wherein the baseline profile data set is related to the multiple sclerosis or inflammatory conditions related to multiple sclerosis to be evaluated, with the calibrated profile data set being a comparison between the first profile data set and the baseline profile data set, thereby providing evaluation of the multiple sclerosis or inflammatory conditions related to multiple sclerosis of the subject.

In related embodiments, the subject has presumptive signs of multiple sclerosis including at least one of: altered sensory, motor, visual or proprioceptive system with at least one of numbness or weakness in one or more limbs, often occurring on one side of the body at a time or the lower half of the body, partial or complete loss of vision, frequently in one eye at a time and often with pain during eye movement, double vision or blurring of vision, tingling or pain in numb areas of the body, electric-shock sensations that occur with certain head movements, tremor, lack of coordination or unsteady gait, fatigue, dizziness, muscle stiffness or spasticity, slurred speech, paralysis, problems with bladder, bowel or sexual function, and mental changes such as forgetfulness or difficulties with concentration, relative to medical standards, or alternatively, the multiple sclerosis or inflammatory conditions may be related to inflammatory conditions.

In addition, the baseline profile data set may be derived from one or more other samples from the same subject taken under circumstances different from those of the first sample, and the circumstances may be selected from the group consisting of (i) the time at which the first sample is taken, (ii) the site from which the first sample is taken, (iii) the biological condition of the subject when the first sample is taken.

Also, the one or more other samples may be taken over an interval of time that is at least one month between the first sample and the one or more other samples, or taken over an interval of time that is at least twelve months between the first sample and the one or more samples, or they may be taken pre-therapy intervention or post-therapy intervention. In such embodiments, the first sample may be derived from blood and the baseline profile data set may be derived from tissue or body fluid of the subject other than blood. Alternatively, the first sample is derived from tissue or body fluid of the subject and the baseline profile data set is derived from blood.

In other embodiments, the baseline profile data set may be derived from one or more other samples from the same subject, taken when the subject is in a biological condition different from that in which the subject was at the time the first sample was taken, with respect to at least one of age, nutritional history, medical condition, clinical indicator, medication, physical activity, body mass, and environmental exposure, and the baseline profile data set may be derived from one or more other samples from one or more different subjects.

In addition, the one or more different subjects may have in common with the subject at least one of age group, gender, ethnicity, geographic location, nutritional history, medical condition, clinical indicator, medication, physical activity, body mass, and environmental exposure. In other embodiments, a clinical indicator may be used to assess multiple sclerosis or inflammatory conditions related to multiple sclerosis of the one or more different subjects, and may also include interpreting the calibrated profile data set in the context of at least one other clinical indicator, wherein the at least one other clinical indicator is selected from the group consisting of blood chemistry, urinalysis, X-ray or other radiological or metabolic imaging technique, other chemical assays, and physical findings.

In such embodiments, the multiple sclerosis or inflammatory conditions related to multiple sclerosis may be from an autoimmune condition, a microbial infection, a bacterial infection, a eukaryotic parasitic infection, a viral infection, a fungal infection, or alternatively, the multiple sclerosis or inflammatory conditions related to multiple sclerosis may be from systemic inflammatory response syndrome (SIRS), from bacteremia, viremia, fungemia, or septicemia due to any class of microbe.

In yet other embodiments, the function is a mathematical function and is other than a simple difference, including a second function of the ratio of the corresponding member of first profile data set to the corresponding member of the baseline profile data set, or a logarithmic function. In related embodiments, each member of the calibrated profile data set has biological significance.-if it has a value differing by more than an amount D, where D=F(1.1)−F(0.9), and F is the second function. In such embodiments, the first sample is obtained and the first profile data set quantified at a first location, and the calibrated profile data set is produced using a network to access a database stored on a digital storage medium in a second location, wherein the database may be updated to reflect the first profile data set quantified from the sample. Additionally, using a network may include accessing a global computer network.

In related embodiments, the quantitative measure is determined by amplification, and the measurement conditions are such that efficiencies of amplification for all constituents differ by less than approximately 2 percent, or alternatively by less than approximately 1 percent.

Still another embodiment is a method of providing an index that is indicative of multiple sclerosis or inflammatory conditions related to multiple sclerosis of a subject based on a first sample from the subject, the first sample providing a source of RNAs, the method comprising deriving from the first sample a profile data set, the profile data set including a plurality of members, each member being a quantitative measure of the amount of a distinct RNA constituent in a panel of constituents selected so that measurement of the constituents is indicative of the presumptive signs of a systemic infection, the panel including at least two of the constituents of the Gene Expression Panel of Table 1. In deriving the profile data set, such measure for each constituent is achieved under measurement conditions that are substantially repeatable, at least one measure from the profile data set is applied to an index function that provides a mapping from at least one measure of the profile data set into one measure of the presumptive signs of a systemic infection, so as to produce an index pertinent to the multiple sclerosis or inflammatory conditions related to multiple sclerosis of the subject.

In addition, the subject may have presumptive signs of multiple sclerosis including at least one of: altered sensory, motor, visual or proprioceptive system with at least one of numbness or weakness in one or more limbs, often occurring on one side of the body at a time or the lower half of the body, partial or complete loss of vision, frequently in one eye at a time and often with pain during eye movement, double vision or blurring of vision, tingling or pain in numb areas of the body, electric-shock sensations that occur with certain head movements, tremor, lack of coordination or unsteady gait, fatigue, dizziness, muscle stiffness or spasticity, slurred speech, paralysis, problems with bladder, bowel or sexual function, and mental changes such as forgetfulness or difficulties with concentration, relative to medical standards, or alternatively, the multiple sclerosis or inflammatory conditions may be related to inflammatory conditions.

In related embodiments, the index function is constructed as a linear sum of terms having the form: I=ΣCᵢMᵢ^{P(i)}, wherein I is the index, Mᵢ is the value of the member i of the profile data set, Cᵢ is a constant, and P(i) is a power to which Mᵢ is raised, the sum being formed for all integral values of i up to the number of members in the data set. In addition, the values Cᵢ and P(i) are determined using statistical techniques, such as latent class modeling, to correlate data, including clinical, experimentally derived, and any other data pertinent to the presumptive signs of a systemic infection. In alternative embodiments, there is provided a normative value of the index function, determined with respect to a relevant set of subjects, so that the index may be interpreted in relation to the normative value, wherein the normative value may include constructing the index function so that the normative value is approximately 1, alternatively so that the normative value is approximately 0 and deviations in the index function from 0 are expressed in standard deviation units. In still other embodiments, the relevant set of subjects has in common a property that is at least one of age group, gender, ethnicity, geographic location, nutritional history, medical condition, clinical indicator, medication, physical activity, body mass, and environmental exposure, or alternatively has in common a property that is at least one of age group, gender, ethnicity, geographic location, nutritional history, medical condition, clinical indicator, medication, physical activity, body mass, and environmental exposure.

In other embodiments, a clinical indicator may be used to assess the multiple sclerosis or inflammatory conditions related to multiple sclerosis of the relevant set of subjects by interpreting the calibrated profile data set in the context of at least one other clinical indicator, wherein the at least one other clinical indicator is selected from the group consisting of blood chemistry, urinalysis, X-ray or other radiological or metabolic imaging technique, other chemical assays, and physical findings. In addition, the quantitative measure may be determined by amplification, the measurement conditions being such that efficiencies of amplification for all constituents differ by less than approximately 2 percent, or they differ by less than approximately 1 percent, and the measurement conditions that are substantially repeatable are within a degree of repeatability of better than five percent, or within a degree of repeatability of better than three percent.

In such embodiments, the multiple sclerosis or inflammatory conditions related to multiple sclerosis being evaluated are with respect to a localized tissue of the subject and the first sample is derived from tissue or fluid of a type distinct from that of the localized tissue, wherein the multiple sclerosis or inflammatory conditions related to multiple sclerosis are from a microbial infection, more particularly a bacterial infection, still more particularly a eukaryotic parasitic infection, a viral infection, a fungal infection or from a systemic inflammatory response syndrome (SIRS).

Other embodiments provide a method of providing an index, further comprising deriving from at least one other sample at least one other profile data set, the at least one other profile data set including a plurality of members, each being a quantitative measure of the amount of a distinct RNA constituent in a panel of constituents selected so that measurement of the constituents is indicative of the presumptive signs of a systemic infection, wherein the at least one other sample is from the same subject, taken under circumstances different from those of the first sample with respect to at least one of time, nutritional history, medical condition, clinical indicator, medication, physical activity, body mass, and environmental exposure, and applying at least one measure from the at least one other profile data set to an index function that provides a mapping from the at least one measure of the at least one other profile data set into one measure of the multiple sclerosis or inflammatory conditions related to multiple sclerosis under different circumstances, so as to produce at least one other index pertinent to the multiple sclerosis or inflammatory conditions related to multiple sclerosis of the subject under circumstances different from those of the first sample.

Related embodiments include providing an index wherein the index function has 2, 3, 4, or 5 components including disease status, disease severity, or disease course. In addition, the index function may be constructed as a linear sum of terms having the form: I=ΣCᵢMᵢ^{P(i)}, wherein I is the index, Mᵢ is the value of the member i of the profile data set, Cᵢ is a constant, and P(i) is a power to which Mᵢ is raised, the sum being formed for all integral values of i up to the number of members in the data set, wherein the values Cᵢ and P(i) are determined using statistical techniques, such as latent class modeling, to correlate data, including clinical, experimentally derived, and any other data pertinent to the presumptive signs of a systemic infection.

Alternatively, a normative value of the index function is provided, determined with respect to a relevant set of subjects, so that the at least one other index may be interpreted in relation to the normative value, wherein providing the normative value includes constructing the index function so that the normative value is approximately 1, or so that the normative value is approximately 0 and deviations in the index function from 0 are expressed in standard deviation units. Such embodiments may also include using a clinical indicator to assess multiple sclerosis or inflammatory conditions related to multiple sclerosis of the relevant set of subjects by interpreting the calibrated profile data set in the context of at least one other clinical indicator selected from the group consisting of blood chemistry, urinalysis, X-ray or other radiological or metabolic imaging technique, other chemical assays, and physical findings.

As in other embodiments, the quantitative measure is determined by amplification, and the measurement conditions are such that efficiencies of amplification for all constituents differ by less than approximately 2 percent, or differ by less than approximately 1 percent, and the measurement conditions that are substantially repeatable are within a degree of repeatability of better than five percent or within a degree of repeatability of better than three percent.

In addition, the multiple sclerosis or inflammatory conditions related to multiple sclerosis are with respect to a localized tissue of the subject and the first sample is derived from tissue or fluid of a type distinct from that of the localized tissue.

Still other embodiments include a method for providing an index wherein the multiple sclerosis or inflammatory conditions related to multiple sclerosis are from an autoimmune condition, a microbial infection, a bacterial infection, a viral infection, a fungal infection, a eukaryotic parasite infection, or from systemic inflammatory response syndrome (SIRS) and the panel of constituents includes at least two constituents of Table 1.

Another embodiment provides a method for evaluating multiple sclerosis or inflammatory conditions related to multiple sclerosis of a subject based on a first sample from the subject, the first sample providing a source of RNAs, the method comprising deriving from the first sample a first profile data set, the first profile data set including a plurality of members, each member being a quantitative measure of the amount of a distinct RNA constituent in a panel of constituents selected so that measurement of the constituents enables evaluation of the multiple sclerosis or inflammatory conditions related to multiple sclerosis wherein such measure for each constituent is obtained under measurement conditions that are substantially repeatable. The method also includes producing a calibrated profile data set for the panel, wherein each member of the calibrated profile data set is a function of a corresponding member of the first profile data set and a corresponding member of a baseline profile data set for the panel, wherein each member of the baseline profile data set is a normative measure determined with respect to a relevant set of subjects of the amount of one of the constituents in the panel and the baseline profile data set is related to the multiple sclerosis or inflammatory conditions related to multiple sclerosis to be evaluated, and the calibrated profile data set is a comparison between the first profile data set and the baseline profile data set, thereby providing evaluation of the multiple sclerosis or inflammatory conditions related to multiple sclerosis of the subject.

In such an embodiment, the subject may have presumptive signs of multiple sclerosis including at least one of: altered sensory, motor, visual or proprioceptive system with at least one of numbness or weakness in one or more limbs, often occurring on one side of the body at a time or the lower half of the body, partial or complete loss of vision, frequently in one eye at a time and often with pain during eye movement, double vision or blurring of vision, tingling or pain in numb areas of the body, electric-shock sensations that occur with certain head movements, tremor, lack of coordination or unsteady gait, fatigue, dizziness, muscle stiffness or spasticity, slurred speech, paralysis, problems with bladder, bowel or sexual function, and mental changes such as forgetfulness or difficulties with concentration, relative to medical standards, or the multiple sclerosis or inflammatory conditions may be related to inflammatory conditions.

Additionally, the relevant set of subjects is a set of healthy subjects having in common a property that is at least one of age group, gender, ethnicity, geographic location, nutritional history, medical condition, clinical indicator, medication, physical activity, body mass, and environmental exposure. As with other embodiments, the quantitative measure is determined by amplification, and the measurement conditions are such that efficiencies of amplification for all constituents differ by less than approximately 2 percent, or they differ by less than approximately 1 percent, and the measurement conditions are substantially repeatable within a degree of repeatability of better than five percent or within a degree of repeatability of better than three percent.

In such embodiments, the multiple sclerosis or inflammatory conditions related to multiple sclerosis being evaluated is with respect to a localized tissue of the subject and the first sample is derived from tissue or fluid of a type distinct from that of the localized tissue and the profile data set may be stored in a digital storage medium, including storing it as a record in a database. In addition, the baseline profile data set is derived from one or more other samples from the same subject taken under circumstances different from those of the first sample, wherein the one or more other samples are taken pre-therapy intervention or alternatively taken post-therapy intervention, or the one or more other samples are taken over an interval of time that is at least one month between an initial sample and the sample, or at least twelve months between an initial sample and the sample. Also, the first sample is derived from blood and the baseline profile data set is derived from tissue or body fluid of the subject other than blood, or alternatively, the first sample is derived from tissue or body fluid of the subject and the baseline profile data set is derived from blood.

Yet another embodiment provides a method for evaluating multiple sclerosis or inflammatory conditions related to multiple sclerosis of a subject based on a first sample from the subject and a second sample from a defined population of indicator cells, the samples providing a source of RNAs, the method comprising applying the first sample or a portion thereof to the defined population of indicator cells. The method also includes deriving from the second sample a first profile data set, the first profile data set including a plurality of members, each member being a quantitative measure of the amount of a distinct RNA or protein constituent in a panel of constituents selected so that measurement of the constituents enables measurement of the presumptive signs of a systemic infection, wherein such measure for each constituent is obtained under measurement conditions that are substantially repeatable, and also includes producing a calibrated profile data set for the panel, wherein each member of the calibrated profile data set is a function of a corresponding member of the first profile data set and a corresponding member of a baseline profile data set for the panel, wherein each member of the baseline data set is a normative measure determined with respect to a relevant set of subjects of the amount of one of the constituents in the panel and wherein the baseline profile data set is related to the multiple sclerosis or inflammatory conditions related to multiple sclerosis to be evaluated, the calibrated profile data set being a comparison between the first profile data set and the baseline profile data set, thereby providing evaluation of the multiple sclerosis or inflammatory conditions related to multiple sclerosis of the subject.

In related embodiments, the subject may have presumptive signs of multiple sclerosis including at least one of: altered sensory, motor, visual or proprioceptive system with at least one of numbness or weakness in one or more limbs, often occurring on one side of the body at a time or the lower half of the body, partial or complete loss of vision, frequently in one eye at a time and often with pain during eye movement, double vision or blurring of vision, tingling or pain in numb areas of the body, electric-shock sensations that occur with certain head movements, tremor, lack of coordination or unsteady gait, fatigue, dizziness, muscle stiffness or spasticity, slurred speech, paralysis, problems with bladder, bowel or sexual function, and mental changes such as forgetfulness or difficulties with concentration, relative to medical standards, or alternatively, the multiple sclerosis or inflammatory conditions may be related to inflammatory conditions.

In addition, the relevant set of subjects has in common a property that is at least one of age group, gender, ethnicity, geographic location, nutritional history, medical condition, clinical indicator, medication, physical activity, body mass, and environmental exposure. Additionally, a clinical indicator may be used to assess multiple sclerosis or inflammatory conditions related to multiple sclerosis of the relevant set of subjects by interpreting the calibrated profile data set in the context of at least one other clinical indicator, wherein the at least one other clinical indicator is selected from the group consisting of blood chemistry, urinalysis, X-ray or other radiological or metabolic imaging technique, other chemical assays, and physical findings.

As with other embodiments, the quantitative measure is determined by amplification, and the measurement conditions are such that efficiencies of amplification for all constituents differ by less than approximately 2 percent, or they differ by less than approximately 1 percent, and the measurement conditions are substantially repeatable within a degree of repeatability of better than five percent, or within a degree of repeatability of better than three percent. Also, the multiple sclerosis being evaluated is with respect to a localized tissue of the subject and the first sample is derived from tissue or fluid of a type distinct from that of the localized tissue, and the multiple sclerosis or inflammatory conditions related to multiple sclerosis is a microbial infection.

In related embodiments, the baseline profile data set is derived from one or more other samples from the same subject taken under circumstances different from those of the first sample, wherein the one or more other samples are taken pre-therapy intervention, or are taken post-therapy intervention, or are taken over an interval of time that is at least one month between an initial sample and the sample, or are taken over an interval of time that is at least twelve months between an initial sample and the sample. In such embodiments, the first sample is derived from blood and the baseline profile data set is derived from tissue or body fluid of the subject other than blood, or the first sample is derived from tissue or body fluid of the subject and the baseline profile data set is derived from blood.

In another embodiment of the invention, a method for evaluating multiple sclerosis or inflammatory conditions related to multiple sclerosis of a target population of cells affected by a first agent, based on a sample from the target population of cells to which the first agent has been administered, the sample providing a source of RNAs, is presented. The method comprises deriving from the sample a first profile data set, the first profile data set including a plurality of members, each member being a quantitative measure of the amount of a distinct RNA constituent in a panel of constituents selected so that measurement of the constituents enables evaluation of the multiple sclerosis or inflammatory conditions related to multiple sclerosis affected by the first agent, wherein such measure for each constituent is obtained under measurement conditions that are substantially repeatable; and producing a calibrated profile data set for the panel, wherein each member of the calibrated profile data set is a function of a corresponding member of the first profile data set and a corresponding member of a baseline profile data set for the panel, wherein each member of the baseline data set is a normative measure determined with respect to a relevant set of target populations of cells of the amount of one of the constituents in the panel, and wherein the baseline profile data set is related to the multiple sclerosis or inflammatory conditions related to multiple sclerosis to be evaluated, the calibrated profile data set being a comparison between the first profile data set and the baseline profile data set, thereby providing an evaluation of the multiple sclerosis or inflammatory conditions related to multiple sclerosis of the target population of cells affected by the first agent.

The target population of cells may have presumptive signs of a systemic infection including at least one of: elevated white blood cell count, elevated temperature, elevated heart rate, and elevated or reduced blood pressure, relative to medical standards. The multiple sclerosis or inflammatory conditions related to multiple sclerosis may be related to inflammatory conditions arising from at least one of: an autoimmune condition, an injury, blunt trauma, surgery, a microbial infection, a bacterial infection, a viral infection, a fungal infection, a eukaryotic parasite infection, or from systemic inflammatory response syndrome (SIRS). The relevant set of target populations of cells may be a set of healthy target populations of cells. Alternatively, the relevant set of target populations of cells may have in common a property that is at least one of age group, gender, ethnicity, geographic location, nutritional history, medical condition, clinical indicator, medication, physical activity, body mass, and environmental exposure. In such a case, a clinical indicator may be used to assess multiple sclerosis or inflammatory conditions related to multiple sclerosis of the relevant set of target populations of cells, and the method further comprises interpreting the calibrated profile data set in the context of at least one other clinical indicator; the at least one other clinical indicator may be selected from the group consisting of blood chemistry, urinalysis, X-ray or other radiological or metabolic imaging technique, other chemical assays, and physical findings. The quantitative measure may be determined by amplification, and the measurement conditions are such that efficiencies of amplification for all constituents differ by less than approximately 2 percent, or alternatively, less than approximately 1 percent. The measurement conditions that are substantially repeatable may be within a degree of repeatability of better than five percent, or alternatively better than three percent. Also, the multiple sclerosis or inflammatory conditions related to multiple sclerosis being evaluated may be with respect to a localized tissue of the subject and the first sample is derived from tissue or fluid of a type distinct from that of the localized tissue. The multiple sclerosis or inflammatory conditions related to multiple sclerosis may be from an autoimmune condition, a microbial infection, a bacterial infection, a eukaryotic parasitic infection, a viral infection, a fungal infection, systemic inflammatory response syndrome (SIRS), bacteremia, viremia, fungemia, or septicemia due to any class of microbe. A related embodiment of the method may further comprise storing the profile data set in a digital storage medium. Storing the profile data set may include storing it as a record in a database. The embodiment may include the limitations that the first sample is derived from blood and the baseline profile data set is derived from tissue or body fluid of the subject other than blood. Alternatively, the first sample may be derived from tissue or body fluid of the subject and the baseline profile data set is derived from blood. As well, the baseline profile data set may be derived from one or more other samples from the same subject taken under circumstances different from those of the first sample. Such one or more other samples may be taken pre-therapy intervention, post-therapy intervention, or over an interval of time that is at least one month between an initial sample and the sample.

Other embodiments of the invention are directed toward a method for evaluating multiple sclerosis or inflammatory conditions related to multiple sclerosis of a target population of cells affected by a first agent in relation to the multiple sclerosis or inflammatory conditions related to multiple sclerosis of the target population of cells affected by a second agent, based on a first sample from the target population cells to which the first agent has been administered and a second sample from the target population of cells to which the second agent has been administered, the samples providing a source of RNAs. Such a method includes the steps of deriving from the first sample a first profile data set and from the second sample a second profile data set, the first and second profile data sets each including a plurality of members, each member being a quantitative measure of the amount of a distinct RNA constituent in a panel of constituents selected so that measurement of the constituents enables evaluation of the multiple sclerosis or inflammatory conditions related to multiple sclerosis affected by the first agent in relation to the second agent, wherein such measure for each constituent is obtained under measurement conditions that are substantially repeatable; and producing a first calibrated profile data set and a second calibrated profile data set for the panel, wherein (i) each member of the first calibrated profile data set is a function of a corresponding member of the first profile data set and a corresponding member of a baseline profile data set for the panel, and (ii) each member of the second calibrated profile data set is a function of a corresponding member of the second profile data set and a corresponding member of the baseline profile data set, wherein each member of the baseline data set is a normative measure, determined with respect to a relevant set of subjects, of the amount of one of the constituents in the panel, and wherein the baseline profile data set is related to the multiple sclerosis or inflammatory conditions related to multiple sclerosis to be evaluated, the first and second calibrated profile data sets being a comparison between the first profile data set and the baseline profile set and a comparison between the second profile data set and the baseline profile data set, thereby providing an evaluation of the multiple sclerosis or inflammatory conditions related to multiple sclerosis of the target population of cells affected by the first agent in relation to the multiple sclerosis or inflammatory conditions related to multiple sclerosis of the target population of cells affected by the second agent. The target population of cells may have presumptive signs of a systemic infection including at least one of: elevated white blood cell count, elevated temperature, elevated heart rate, and elevated or reduced blood pressure, relative to medical standards. As well, the target population of cells may have presumptive signs of a systemic infection that are related to inflammatory conditions arising from at least one of: an autoimmune condition, an injury, blunt trauma, surgery, a microbial infection, a bacterial infection, a viral infection, a fungal infection, a eukaryotic parasite infection, or from systemic inflammatory response syndrome (SIRS). The first agent may be a first drug and the second agent may be a second drug. Alternatively, the first agent is a drug and the second agent is a complex mixture or a nutriceutical. The quantitative measure may be determined by amplification, and the measurement conditions are such that efficiencies of amplification for all constituents differ by less than approximately 2 percent, or alternatively by less than approximately 1 percent. The measurement conditions that are substantially repeatable may be within a degree of repeatability of better than five percent, or alternatively better than three percent. The multiple sclerosis or inflammatory conditions related to multiple sclerosis being evaluated may be with respect to a localized tissue of the subject and the first sample is derived from tissue or fluid of a type distinct from that of the localized tissue. The multiple sclerosis or inflammatory conditions related to multiple sclerosis may be from an autoimmune condition, a microbial infection, bacterial infection, a eukaryotic parasitic infection, a viral infection, a fungal infection, systemic inflammatory response syndrome (SIRS), bacteremia, viremia, fungemia, or septicemia due to any class of microbe. This method may further include the step of storing the first and second profile data sets in a digital storage medium. The first and second profile data sets may include storing each data set as a record in a database. The baseline profile data set may be derived from one or more other samples from the same subject taken under circumstances different from those of the first sample, or alternatively different from those of the second sample. The first sample may be derived from blood and the baseline profile data set may be derived from tissue or body fluid of the subject other than blood. The first sample may be derived from tissue or body fluid of the subject and the baseline profile data set may be derived from blood.

In yet another embodiment of the invention, a method of providing an index that is indicative of an inflammatory condition of a subject with presumptive signs of a systemic infection, based on a first sample from the subject, the first sample providing a source of RNAs, is presented. The method comprises deriving from the first sample a profile data set, the profile data set including a plurality of members, each member being a quantitative measure of the amount of a distinct RNA constituent in a panel of constituents selected so that measurement of the constituents is indicative of the inflammatory condition, the panel including at least two of the constituents of the Gene Expression Panel of Table 1; and in deriving the profile data set, achieving such measure for each constituent under measurement conditions that are substantially repeatable; applying at least one measure from the profile data set to an index function that provides a mapping from at least one measure of the profile data set into at least one measure of the inflammatory condition, so as to produce an index pertinent to the inflammatory condition of the sample; wherein the index function uses data from a baseline profile data set for the panel, each member of the baseline data set being a normative measure, determined with respect to a relevant set of subjects, of the amount of one of the constituents in the panel, wherein the baseline data set is related to the inflammatory condition to be evaluated. The subject may have presumptive signs of a systemic infection including at least one of: elevated white blood cell count, elevated temperature, elevated heart rate, and elevated or reduced blood pressure, relative to medical standards. Alternatively, the presumptive signs of a systemic infection are related to inflammatory conditions arising from at least one of: an autoimmune condition, an injury, blunt trauma, surgery, a microbial infection, a bacterial infection, a viral infection, a fungal infection, a eukaryotic parasite infection, or from systemic inflammatory response syndrome (SIRS). The at least one measure of the profile data set that is applied to the index function may be 2, 3, 4, or 5.

Still other embodiments provide a method of using an index to direct therapy intervention in a subject with multiple sclerosis or inflammatory conditions related to multiple sclerosis, the method comprising providing an index according to any of the above-discussed embodiments, comparing the index to a normative value of the index, determined with respect to a relevant set of subjects to obtain a difference, and using the difference between the index and the normative value for the index to direct therapy intervention, wherein therapy intervention is microbe-specific therapy, or is bacteria-specific therapy, or is fungus-specific therapy, or is virus-specific therapy, or is eukaryotic parasite-specific therapy.

Another embodiment provides a method for differentiating a type of pathogen within a class of pathogens of interest in a subject with multiple sclerosis or inflammatory conditions related to multiple sclerosis, based on at least one sample from the subject, the sample providing a source of RNA, the method comprising: determining at least one profile data set for the subject, comparing the profile data set to at least one baseline profile data set, determined with respect to at least one relevant set of samples within the class of pathogens of interest to obtain a difference, and using the difference to differentiate the type of pathogen in the at least one profile data set for the subject from the class of pathogen in the at least one baseline profile data set, wherein the class of pathogens is microbial. Alternatively, the class of pathogens is bacterial and the difference is used to differentiate a Gram(+) bacterial pathogen from a Gram(−) bacterial pathogen. Alternatively, the class of pathogens is fungal and the difference is used to differentiate an acute Candida pathogen from a chronic Candida pathogen. More particularly, the class of pathogens is viral and the difference is used to differentiate a DNA viral pathogen from an RNA viral pathogen, or the class of pathogens is viral and the difference is used to differentiate a rhinovirus pathogen from an influenza pathogen. Still more particularly, the class of pathogens is eukaryotic parasites and the difference is used to differentiate a plasmodium parasite pathogen from a trypanosomal pathogen.

Yet another embodiment provides a method of using an index for differentiating a type of pathogen within a class of pathogens of interest in a subject with multiple sclerosis or inflammatory conditions related to multiple sclerosis, based on at least one sample from the subject, the method comprising providing at least one index according to any of the above disclosed embodiments for the subject, comparing the at least one index to at least one normative value of the index, determined with respect to at least one relevant set of subjects to obtain at least one difference, and using the at least one difference between the at least one index and the at least one normative value for the index to differentiate the type of pathogen from the class of pathogen.

### BRIEF DESCRIPTION OF THE DRAWINGS

The foregoing features of the invention will be more readily understood by reference to the following detailed description, taken with reference to the accompanying drawings, in which:

FIG. 1A shows the results of assaying 24 genes from the Source Inflammation Gene Panel (shown in Table 1) on eight separate days during the course of optic neuritis in a single male subject.

1B illustrates use of an inflammation index in relation to the data of FIG. 1A, in accordance with an embodiment of the present invention.

FIG. 2 is a graphical illustration of the same inflammation index calculated at 9 different, significant clinical milestones.

FIG. 3 shows the effects of single dose treatment with 800 mg of ibuprofen in a single donor as characterized by the index.

FIG. 4 shows the calculated acute inflammation index displayed graphically for five different conditions.

FIG. 5 shows a Viral Response Index for monitoring the progress of an upper respiratory infection (URI).

FIGS. 6 and 7 compare two different populations using Gene Expression Profiles (with respect to the 48 loci of the Inflammation Gene Expression Panel of Table 1).

FIG. 8 compares a normal population with a rheumatoid arthritis population derived from a longitudinal study.

FIG. 9 compares two normal populations, one longitudinal and the other cross sectional.

FIG. 10 shows the shows gene expression values for various individuals of a normal population.

FIG. 11 shows the expression levels for each of four genes (of the Inflammation Gene Expression Panel of Table 1), of a single subject, assayed monthly over a period of eight months.

FIGS. 12 and 13 similarly show in each case the expression levels for each of 48 genes (of the Inflammation Gene Expression Panel of Table 1), of distinct single subjects (selected in each case on the basis of feeling well and not taking drugs), assayed, in the case of FIG. 12 weekly over a period of four weeks, and in the case of FIG. 13 monthly over a period of six months.

FIG. 14 shows the effect over time, on inflammatory gene expression in a single human subject., of the administration of an anti-inflammatory steroid, as assayed using the Inflammation Gene Expression Panel of Table 1.

FIG. 15, in a manner analogous to FIG. 14, shows the effect over time, via whole blood samples obtained from a human subject, administered a single dose of prednisone, on expression of 5 genes (of the Inflammation Gene Expression Panel of Table 1).

FIG. 16 also shows the effect over time, on inflammatory gene expression in a single human subject suffering from rheumatoid arthritis, of the administration of a TNF-inhibiting compound, but here the expression is shown in comparison to the cognate locus average previously determined (in connection with FIGS. 6 and 7) for the normal (i.e., undiagnosed, healthy) population.

FIG. 17A further illustrates the consistency of inflammatory gene expression in a population.

FIG. 17B shows the normal distribution of index values obtained from an undiagnosed population.

FIG. 17C illustrates the use of the same index as FIG. 17B, where the inflammation median for a normal population has been set to zero and both normal and diseased subjects are plotted in standard deviation units relative to that median.

FIG. 18 plots, in a fashion similar to that of FIG. 17A, Gene Expression Profiles, for the same 7 loci as in FIG. 17A, two different (responder v. non-responder) 6-subject populations of rheumatoid arthritis patients.

FIG. 19 thus illustrates use of the inflammation index for assessment of a single subject suffering from rheumatoid arthritis, who has not responded well to traditional therapy with methotrexate.

FIG. 20 similarly illustrates use of the inflammation index for assessment of three subjects suffering from rheumatoid arthritis, who have not responded well to traditional therapy with methotrexate.

Each of FIGS. 21-23 shows the inflammation index for an international group of subjects, suffering from rheumatoid arthritis, undergoing three separate treatment regimens.

FIG. 24 illustrates use of the inflammation index for assessment of a single subject suffering from inflammatory bowel disease.

FIG. 25 shows Gene Expression Profiles with respect to 24 loci (of the Inflammation Gene Expression Panel of Table 1) for whole blood treated with Ibuprofen in vitro in relation to other non-steroidal anti-inflammatory drugs (NSAIDs).

FIG. 26 illustrates how the effects of two competing anti-inflammatory compounds can be compared objectively, quantitatively, precisely, and reproducibly.

FIGS. 27 through 41 illustrate the use of gene expression panels in early identification and monitoring of infectious disease.

FIG. 27 uses a novel bacterial Gene Expression Panel of 24 genes, developed to discriminate various bacterial conditions in a host biological system.

FIG. 28 shows differential expression for a single locus, IFNG, to LTA derived from three distinct sources: S. pyogenes, B. subtilis, and S. aureus.

FIGS. 29 and 30 show the response after two hours of the Inflammation 48A and 48B loci respectively (discussed above in connection with FIGS. 6 and 7 respectively) in whole blood to administration of a Gram-positive and a Gram-negative organism.

FIGS. 31 and 32 correspond to FIGS. 29 and 30 respectively and are similar to them, with the exception that the monitoring here occurs 6 hours after administration.

FIG. 33 compares the gene expression response induced by E. coli and by an organism-free E. coli filtrate.

FIG. 34 is similar to FIG. 33, but here the compared responses are to stimuli from E. coli filtrate alone and from E. coli filtrate to which has been added polymyxin B.

FIG. 35 illustrates the gene expression responses induced by S. aureus at 2, 6, and 24 hours after administration.

FIGS. 36 through 41 compare the gene expression induced by E. coli and S. aureus under various concentrations and times.

FIG. 42 illustrates application of a statistical T-test to identify potential members of a signature gene expression panel that is capable of distinguishing between normal subjects and subjects suffering from unstable rheumatoid arthritis.

FIG. 43 illustrates, for a panel of 17 genes, the expression levels for 8 patients presumed to have bacteremia.

FIG. 44 illustrates application of a statistical T-test to identify potential members of a signature gene expression panel that is capable of distinguishing between normal subjects and subjects suffering from bacteremia

FIG. 45 illustrates application of an algorithm (shown in the figure), providing an index pertinent to rheumatoid arthritis (RA) as applied respectively to normal subjects, RA patients, and bacteremia patients.

FIG. 46 illustrates application of an algorithm (shown in the figure), providing an index pertinent to bacteremia as applied respectively to normal subjects, rheumatoid arthritis patients, and bacteremia patients.

FIG. 47 illustrates, for a panel of 47 genes selected genes from Table 1, the expression levels for a patient suffering from multiple sclerosis on dates May 22, 2002 (no treatment), May 28, 2002 (after 5 mg prednisone given on May 22), and Jul. 15, 2002 (after 100 mg prednisone given on May 28, tapering to 5 mg within one week).

### DETAILED DESCRIPTION OF SPECIFIC EMBODIMENTS

### DEFINITIONS

The following terms shall have the meanings indicated unless the context otherwise requires:

“Algorithm” is a set of rules for describing a biological condition. The rule set may be defined exclusively algebraically but may also include alternative or multiple decision points requiring domain-specific knowledge, expert interpretation or other clinical indicators.

An “agent” is a “composition” or a “stimulus”, as those terms are defined herein, or a combination of a composition and a stimulus.

“Amplification” in the context of a quantitative RT-PCR assay is a function of the number of DNA replications that are tracked to provide a quantitative determination of its concentration. “Amplification” here refers to a degree of sensitivity and specificity of a quantitative assay technique. Accordingly, amplification provides a measurement of concentrations of constituents that is evaluated under conditions wherein the efficiency of amplification and therefore the degree of sensitivity and reproducibility for measuring all constituents is substantially similar.

A “baseline profile data set” is a set of values associated with constituents of a Gene Expression Panel resulting from evaluation of a biological sample (or population or set of samples) under a desired biological condition that is used for mathematically normative purposes. The desired biological condition may be, for example, the condition of a subject (or population or set of subjects) before exposure to an agent or in the presence of an untreated disease or in the absence of a disease. Alternatively, or in addition, the desired biological condition may be health of a subject or a population or set of subjects. Alternatively, or in addition, the desired biological condition may be that associated with a population or set of subjects selected on the basis of at least one of age group, gender, ethnicity, geographic location, nutritional history, medical condition, clinical indicator, medication, physical activity, body mass, and environmental exposure.

A “set” or “population” of samples or subjects refers to a defined or selected group of samples-or subjects wherein there is an underlying commonality or relationship between the members included in the set or population of samples or subjects.

A “population of cells” refers to any group of cells wherein there is an underlying commonality or relationship between the members in the population of cells, including a group of cells taken from an organism or from a culture of cells or from a biopsy, for example,

A “biological condition” of a subject is the condition of the subject in a pertinent realm that is under observation, and such realm may include any aspect of the subject capable of being monitored for change in condition, such as health, disease including cancer; autoimmune condition; trauma; aging; infection; tissue degeneration; developmental steps; physical fitness; obesity, and mood. As can be seen, a condition in this context may be chronic or acute or simply transient. Moreover, a targeted biological condition may be manifest throughout the organism or population of cells or may be restricted to a specific organ (such as skin, heart, eye or blood), but in either case, the condition may be monitored directly by a sample of the affected population of cells or indirectly by a sample derived elsewhere from the subject. The term “biological condition” includes a “physiological condition”.

“Body fluid” of a subject includes blood, urine, spinal fluid, lymph, mucosal secretions, prostatic fluid, semen, haemolymph or any other body fluid known in the art for a subject.

“Calibrated profile data set” is a function of a member of a first profile data set and a corresponding member of a baseline profile data set for a given constituent in a panel.

A “clinical indicator” is any physiological datum used alone or in conjunction with other data in evaluating the physiological condition of a collection of cells or of an organism. This term includes pre-clinical indicators.

A “composition” includes a chemical compound, a nutriceutical, a pharmaceutical, a homeopathic formulation, an allopathic formulation, a naturopathic formulation, a combination of compounds, a toxin, a food, a food supplement, a mineral, and a complex mixture of substances, in any physical state or in a combination of physical states.

To “derive” a profile data set from a sample includes determining a set of values associated with constituents of a Gene Expression Panel either (i) by direct measurement of such constituents in a biological sample or (ii) by measurement of such constituents in a second biological sample that has been exposed to the original sample or to matter derived from the original sample.

“Distinct RNA or protein constituent” in a panel of constituents is a distinct expressed product of a gene, whether RNA or protein. An “expression” product of a gene includes the gene product whether RNA or protein resulting from translation of the messenger RNA.

A “Gene Expression Panel” is an experimentally verified set of constituents, each constituent being a distinct expressed product of a gene, whether RNA or protein, wherein constituents of the set are selected so that their measurement provides a measurement of a targeted biological condition.

A “Gene Expression Profile” is a set of values associated with constituents of a Gene Expression Panel resulting from evaluation of a biological sample (or population or set of samples).

A “Gene Expression Profile Inflammatory Index” is the value of an index function that provides a mapping from an instance of a Gene Expression Profile into a single-valued measure of inflammatory condition.

The “health” of a subject includes mental, emotional, physical, spiritual, allopathic, naturopathic and homeopathic condition of the subject.

“Index” is an arithmetically or mathematically derived numerical characteristic developed for aid in simplifying or disclosing or informing the analysis of more complex quantitative information. A disease or population index may be determined by the application of a specific algorithm to a plurality of subjects or samples with a common biological condition.

“Inflammation” is used herein in the general medical sense of the word and may be an acute or chronic; simple or suppurative; localized or disseminated; cellular and tissue response, initiated or sustained by any number of chemical, physical or biological agents or combination of agents.

“Inflammatory state” is used to indicate the relative biological condition of a subject resulting from inflammation, or characterizing the degree of inflammation

A “large number” of data sets based on a common panel of genes is a number of data sets sufficiently large to permit a statistically significant conclusion to be drawn with respect to an instance of a data set based on the same panel.

A “normative” condition of a subject to whom a composition is to be administered means the condition of a subject before administration, even if the subject happens to be suffering from a disease.

A “panel” of genes is a set of genes including at least two constituents.

A “sample” from a subject may include a single cell or multiple cells or fragments of cells or an aliquot of body fluid, taken from the subject, by means including venipuncture, excretion, ejaculation, massage, biopsy, needle aspirate, lavage sample, scraping, surgical incision or intervention or other means known in the art.

A “Signature Profile” is an experimentally verified subset of a Gene Expression Profile selected to discriminate a biological condition, agent or physiological mechanism of action.

A “Signature Panel” is a subset of a Gene Expression Panel, the constituents of which are selected to permit discrimination of a biological condition, agent or physiological mechanism of action.

A “subject” is a cell, tissue, or organism, human or non-human, whether in vivo, ex vivo or in vitro, under observation. When we refer to evaluating the biological condition of a subject based on a sample from the subject, we include using blood or other tissue sample from a human subject to evaluate the human subject's condition; but we also include, for example, using a blood sample itself as the subject to evaluate, for example, the effect of therapy or an agent upon the sample.

A “stimulus” includes (i) a monitored physical interaction with a subject, for example ultraviolet A or B, or light therapy for seasonal affective disorder, or treatment of psoriasis with psoralen or treatment of melanoma with embedded radioactive seeds, other radiation exposure, and (ii) any monitored physical, mental, emotional, or spiritual activity or inactivity of a subject.

“Therapy” includes all interventions whether biological, chemical, physical, metaphysical, or combination of the foregoing, intended to sustain or alter the monitored biological condition of a subject.

The PCT patent application publication number WO 01/25473, published Apr. 12, 2001, entitled “Systems and Methods for Characterizing a Biological Condition or Agent Using Calibrated Gene Expression Profiles,” filed for an invention by inventors herein, and which is herein incorporated by reference, discloses the use of Gene Expression Panels for the evaluation of (i) biological condition (including with respect to health and disease) and (ii) the effect of one or more agents on biological condition (including with respect to health, toxicity, therapeutic treatment and drug interaction).

In particular, Gene Expression Panels may be used for measurement of therapeutic efficacy of natural or synthetic compositions or stimuli that may be formulated individually or in combinations or mixtures for a range of targeted biological conditions; prediction of toxicological effects and dose effectiveness of a composition or mixture of compositions for an individual or for a population or set of individuals or for a population of cells; determination of how two or more different agents administered in a single treatment might interact so as to detect any of synergistic, additive, negative, neutral or toxic activity; performing pre-clinical and clinical trials by providing new criteria for pre-selecting subjects according to informative profile data sets for revealing disease status; and conducting preliminary dosage studies for these patients prior to conducting phase 1 or 2 trials. These Gene Expression Panels may be employed with respect to samples derived from subjects in order to evaluate their biological condition.

A Gene Expression Panel is selected in a manner so that quantitative measurement of RNA or protein constituents in the Panel constitutes a measurement of a biological condition of a subject. In one kind of arrangement, a calibrated profile data set is employed. Each member of the calibrated profile data set is a function of (i) a measure of a distinct constituent of a Gene Expression Panel and (ii) a baseline quantity.

We have found that valuable and unexpected results may be achieved when the quantitative measurement of constituents is performed under repeatable conditions (within a degree of repeatability of measurement of better than twenty percent, and preferably five percent or better, and more preferably three percent or better). For the purposes of this description and the following claims, we regard a degree of repeatability of measurement of better than twenty percent as providing measurement conditions that are “substantially repeatable”. In particular, it is desirable that, each time a measurement is obtained corresponding to the level of expression of a constituent in a particular sample, substantially the same measurement should result for the substantially the same level of expression. In this manner, expression levels for a constituent in a Gene Expression Panel may be meaningfully compared from sample to sample. Even if the expression level measurements for a particular constituent are inaccurate (for example, say, 30% too low), the criterion of repeatability means that all measurements for this constituent, if skewed, will nevertheless be skewed systematically, and therefore measurements of expression level of the constituent may be compared meaningfully. In this fashion valuable information may be obtained and compared concerning expression of the constituent under varied circumstances.

In addition to the criterion of repeatability, it is desirable that a second criterion also be satisfied, namely that quantitative measurement of constituents is performed under conditions wherein efficiencies of amplification for all constituents are substantially similar (within one to two percent and typically one percent or less). When both of these criteria are satisfied, then measurement of the expression level of one constituent may be meaningfully compared with measurement of the expression level of another constituent in a given sample and from sample to sample.

Present embodiments relate to the use of an index or algorithm resulting from quantitative measurement of constituents, and optionally in addition, derived from either expert analysis or computational biology (a) in the analysis of complex data sets; (b) to control or normalize the influence of uninformative or otherwise minor variances in gene expression values between samples or subjects; (c) to simplify the characterization of a complex data set for comparison to other complex data sets, databases or indices or algorithms derived from complex data sets; (d) to monitor a biological condition of a subject; (e) for measurement of therapeutic efficacy of natural or synthetic compositions or stimuli that may be formulated individually or in combinations or mixtures for a range of targeted biological conditions; (f) for predictions of toxicological effects and dose effectiveness of a composition or mixture of compositions for an individual or for a population or set of individuals or for a population of cells; (g) for determination of how two or more different agents administered in a single treatment might interact so as to detect any of synergistic, additive, negative, neutral of toxic activity (h) for performing pre-clinical and clinical trials by providing new criteria for pre-selecting subjects according to informative profile data sets for revealing disease status and conducting preliminary dosage studies for these patients prior to conducting phase 1 or 2 trials.

Gene expression profiling and the use of index characterization for a particular condition or agent or both may be used to reduce the cost of phase 3 clinical trials and may be used beyond phase 3 trials; labeling for approved drugs; selection of suitable medication in a class of medications for a particular patient that is directed to their unique physiology; diagnosing or determining a prognosis of a medical condition or an infection which may precede onset of symptoms or alternatively diagnosing adverse side effects associated with administration of a therapeutic agent; managing the health care of a patient; and quality control for different batches of an agent or a mixture of agents.

### The Subject

The methods disclosed here may be applied to cells of humans, mammals or other organisms without the need for undue experimentation by one of ordinary skill in the art because all cells transcribe RNA and it is known in the art how to extract RNA from all types of cells.

### Selecting Constituents of a Gene Expression Panel

The general approach to selecting constituents of a Gene Expression Panel has been described in PCT application publication number WO 01/25473. We have designed and experimentally verified a wide range of Gene Expression Panels, each panel providing a quantitative measure, of biological condition, that is derived from a sample of blood or other tissue. For each panel, experiments have verified that a Gene Expression Profile using the panel's constituents is informative of a biological condition. (We show elsewhere that in being informative of biological condition, the Gene Expression Profile can be used to used, among other things, to measure the effectiveness of therapy, as well as to provide a target for therapeutic intervention.) Table 1, listed below, includes relevant genes which may be selected for a given Gene Expression Panel, such as the Gene Expression Panels provided in various figures:

Table 1. Multiple Sclerosis or Inflammatory Conditions Related to Multiple Sclerosis Gene Expression Panel

In general, panels may be constructed and experimentally verified by one of ordinary skill in the art in accordance with the principles articulated in the present application.

### Design of Assays

We commonly run a sample through a panel in quadruplicate; that is, a sample is divided into aliquots and for each aliquot we measure concentrations of each constituent in a Gene Expression Panel. Over a total of 900 constituent assays, with each assay conducted in quadruplicate, we found an average coefficient of variation, (standard deviation/average)*100, of less than 2 percent, typically less than 1 percent, among results for each assay. This figure is a measure of what we call “intra-assay variability”. We have also conducted assays on different occasions using the same sample material. With 72 assays, resulting from concentration measurements of constituents in a panel of 24 members, and such concentration measurements determined on three different occasions over time, we found an average coefficient of variation of less than 5 percent, typically less than 2 percent. We regard this as a measure of what we call “inter-assay variability”.

We have found it valuable in using the quadruplicate test results to identify and eliminate data points that are statistical “outliers”; such data points are those that differ by a percentage greater, for example, than 3% of the average of all four values and that do not result from any systematic skew that is, greater, for example, than 1%. Moreover, if more than-one data point in a set of four is excluded by this procedure, then all data for the relevant constituent is discarded.

### Measurement of Gene Expression for a Constituent in the Panel

For measuring the amount of a particular RNA in a sample, we have used methods known to one of ordinary skill in the art to extract and quantify transcribed RNA from a sample with respect to a constituent of a Gene Expression Panel. (See detailed protocols below. Also see PCT application publication number WO 98/24935 herein incorporated by reference for RNA analysis protocols). Briefly, RNA is extracted from a sample such as a tissue, body fluid, or culture medium in which a population of cells of a subject might be growing. For example, cells may be lysed and RNA eluted in a suitable solution in which to conduct a DNAse reaction. First strand synthesis may be performed using a reverse transcriptase. Gene amplification, more specifically quantitative PCR assays, can then conducted and the gene of interest size calibrated against a marker such as 18S rRNA (Hirayama et al., Blood 92, 1998: 46-52). Samples are measured in multiple duplicates, for example, 4 replicates. Relative quantitation of the mRNA is determined by the difference in threshhold cycles between the internal control and the gene of interest. In an embodiment of the invention, quantitative PCR is performed using amplification, reporting agents and instruments such as those supplied commercially by Applied Biosystems (Foster City, Calif.). Given a defined efficiency of amplification of target transcripts, the point (e.g., cycle number) that signal from amplified target template is detectable may be directly related to the amount of specific message transcript in the measured sample. Similarly, other quantifiable signals such as fluorescence, enzyme activity, disintegrations per minute, absorbance, etc., when correlated to a known concentration of target templates (e.g., a reference standard curve) or normalized to a standard with limited variability can be used to quantify the number of target templates in an unknown sample.

Although not limited to amplification methods, quantitative gene expression techniques may utilize amplification of the target transcript. Alternatively or in combination with amplification of the target transcript, amplification of the reporter signal may also be used. Amplification of the target template may be accomplished by isothermic gene amplification strategies, or by gene amplification by thermal cycling such as PCR.

It is desirable to obtain a definable and reproducible correlation between the amplified target or reporter and the concentration of starting templates. We have discovered that this objective can be achieved by careful attention to, for example, consistent primer-template ratios and a strict adherence to a narrow permissible level of experimental amplification efficiencies (for example 99.0 to 100% relative efficiency, typically 99.8 to 100% relative efficiency). For example, in determining gene expression levels with regard to a single Gene Expression Profile, it is necessary that all constituents of the panels maintain a similar and limited range of primer template ratios (for example, within a 10-fold range) and amplification efficiencies (within, for example, less than 1%) to permit accurate and precise relative measurements for each constituent. We regard amplification efficiencies as being “substantially similar”, for the purposes of this description and the following claims, if they differ by no more than approximately 10%. Preferably they should differ by less than approximately 2% and more preferably by less than approximately 1%. These constraints should be observed over the entire range of concentration levels to be measured associated with the relevant biological condition. While it is thus necessary for various embodiments herein to satisfy criteria that measurements are achieved under measurement conditions that are substantially repeatable and wherein specificity and efficiencies of amplification for all constituents are substantially similar, nevertheless, it is within the scope of the present invention as claimed herein to achieve such measurement conditions by adjusting assay results that do not satisfy these criteria directly, in such a manner as to compensate for errors, so that the criteria are satisfied after suitable adjustment of assay results.

In practice, we run tests to assure that these conditions are satisfied. For example, we typically design and manufacture a number of primer-probe sets, and determine experimentally which set gives the best performance. Even though primer-probe design and manufacture can be enhanced using computer techniques known in the art, and notwithstanding common practice, we still find that experimental validation is useful. Moreover, in the course of experimental validation, we associate with the selected primer-probe combination a set of features:

The reverse primer should be complementary to the coding DNA strand. In one embodiment, the primer should be located across an intron-exon junction, with not more than three bases of the three-prime end of the reverse primer complementary to the proximal exon. (If more than three bases are complementary, then it would tend to competitively amplify genomic DNA.)

In an embodiment of the invention, the primer probe should amplify cDNA of less than 110 bases in length and should not amplify genomic DNA or transcripts or cDNA from related but biologically irrelevant loci.

A suitable target of the selected primer probe is first strand cDNA, which may be prepared, in one embodiment, is described as follows:

(a) Use of whole blood for ex vivo assessment of a biological condition affected by an agent.

Human blood is obtained by venipuncture and prepared for assay by separating samples for baseline, no stimulus, and stimulus with sufficient volume for at least three time points. Typical stimuli include lipopolysaccharide (LPS), phytohemagglutinin (PHA) and heat-killed staphylococci (HKS) or carrageean and may be used individually (typically) or in combination. The aliquots of heparinized, whole blood are mixed without stimulus and held at 37° C. in an atmosphere of 5% CO2 for 30 minutes. Stimulus is added at varying concentrations, mixed and held loosely capped at 37° C. for 30 min. Additional test compounds may be added at this point and held for varying times depending on the expected pharmacokinetics of the test compound. At defined times, cells are collected by centrifugation, the plasma removed and RNA extracted by various standard means.

Nucleic acids, RNA and or DNA are purified from cells, tissues or fluids of the test population of cells or indicator cell lines. RNA is preferentially obtained from the nucleic acid mix using a variety of standard procedures (or RNA Isolation Strategies, pp. 55-104, in RNA Methodologies, A laboratory guide for isolation and characterization, 2nd edition, 1998, Robert E. Farrell, Jr., Ed., Academic Press), in the present using a filter-based RNA isolation system from Ambion (RNAqueous™, Phenol-free Total RNA Isolation Kit, Catalog #1912, version 9908; Austin, Tex.).

In accordance with one procedure, the whole blood assay for Gene Expression Profiles determination was carried out as follows: Human whole blood was drawn into 10 mL Vacutainer tubes with Sodium Heparin. Blood samples were mixed by gently inverting tubes 4-5 times. The blood was used within 10-15 minutes of draw. In the experiments, blood was diluted 2-fold, i.e. per sample per time point, 0.6 mL whole blood +0.6 mL stimulus. The assay medium was prepared and the stimulus added as appropriate.

A quantity (0.6 mL) of whole blood was then added into each 12×75 mm polypropylene tube. 0.6 mL of 2× LPS (from E. coli serotye 0127:B8, Sigma#L3880 or serotype 055, Sigma #M4005, 10 ng/ml, subject to change in different lots) into LPS tubes was added. Next, 0.6 mL assay medium was added to the “control” tubes with duplicate tubes for each condition. The caps were closed tightly. The tubes were inverted 2-3 times to mix samples. Caps were loosened to first stop and the tubes incubated@37° C., 5% CO2 for 6 hours. At 6 hours, samples were gently mixed to resuspend blood cells, and 1 mL was removed from each tube (using a micropipettor with barrier tip), and transfered to a 2 mL “dolphin” microfuge tube (Costar #3213).

The samples were then centrifuged for 5 min at 500×g, ambient temperature (IEC centrifuge or equivalent, in microfuge tube adapters in swinging bucket), and as much serum from each tube was removed as possible and discarded. Cell pellets were placed on ice; and RNA extracted as soon as possible using an Ambion RNAqueous kit.

(b) Amplification Strategies.

Specific RNAs are amplified using message specific primers or random primers. The specific primers are synthesized from data obtained from public databases (e.g., Unigene, National Center for Biotechnology Information, National Library of Medicine, Bethesda, Md.), including information from genomic and cDNA libraries obtained from humans and other animals. Primers are chosen to preferentially amplify from specific RNAs obtained from the test or indicator samples, see, for example, RT PCR, Chapter 15 in RNA Methodologies, A laboratory guide for isolation and characterization, 2nd edition, 1998,Robert E. Farrell, Jr., Ed., Academic Press; or Chapter 22 pp. 143-151, RNA isolation and characterization protocols, Methods in molecular biology, Volume 86, 1998, R. Rapley and D. L. Manning Eds., Human Press, or 14 in Statistical refinement of primer design parameters, Chapter 5, pp. 55-72, PCR applications: protocols for functional genomics, M. A. Innis, D. H. Gelfand and J. J. Sninsky, Eds., 1999, Academic Press). Amplifications are carried out in either isothermic conditions or using a thermal cycler (for example, a ABI 9600 or 9700 or 7700 obtained from Applied Biosystems, Foster City, Calif.; see Nucleic acid detection methods, pp. 1-24, in Molecular methods for virus detection, D. L. Wiedbrauk and D. H., Farkas, Eds., 1995, Academic Press). Amplified nucleic acids are detected using fluorescent-tagged detection primers (see, for example, Taqman™ PCR Reagent Kit, Protocol, part number 402823 revision A, 1996, Applied Biosystems, Foster City Calif.) that are identified and synthesized from publicly known databases as described for the amplification primers. In the present case, amplified DNA is detected and quantified using the ABI Prism 7700 Sequence Detection System obtained from Applied Biosystems (Foster City, Calif.). Amounts of specific RNAs contained in the test sample or obtained from the indicator cell lines can be related to the relative quantity of fluorescence observed (see for example, Advances in quantitative PCR technology: 5′ nuclease assays, Y. S. Lie and C. J. Petropolus, Current Opinion in Biotechnology, 1998, 9:43-48, or Rapid thermal cycling and PCR kinetics, pp. 211-229, chapter 14 in PCR applications: protocols for functional genomics, M. A. Innis, D. H. Gelfand and J. J. Sninsky, Eds., 1999, Academic Press).

As a particular implementation of the approach described here, we describe in detail a procedure for synthesis of first strand cDNA for use in PCR. This procedure can be used for both whole blood RNA and RNA extracted from cultured cells (i.e. THP-1 cells).

### Materials

1. Applied Biosystems TAQMAN Reverse Transcription Reagents Kit (P/N 808-0234). Kit Components: 10× TaqMan RT Buffer, 25 mM Magnesium chloride, deoxyNTPs mixture, Random Hexamers, RNase Inhibitor, MultiScribe Reverse Transcriptase (50 U/mL) (2) RNase/DNase free water (DEPC Treated Water from Ambion (P/N 9915G), or equivalent)

### Methods

1. Place RNase Inhibitor and MultiScribe Reverse Transcriptase on ice immediately. All other reagents can be thawed at room temperature and then placed on ice.

2. Remove RNA samples from −80° C. freezer and thaw at room temperature and then place immediately on ice.

3. Prepare the following cocktail of Reverse Transcriptase Reagents for each 100 mL RT reaction (for multiple samples, prepare extra cocktail to allow for pipetting error):

| | 1 reaction (mL) | 11X, e.g. 10 samples (mL) |
|---|---|---|
| 10X RT Buffer | 10.0 | 110.0 |
| 25 mM MgCl2 | 22.0 | 242.0 |
| dNTPs | 20.0 | 220.0 |
| Random Hexamers | 5.0 | 55.0 |
| RNAse Inhibitor | 2.0 | 22.0 |
| Reverse Transcriptase | 2.5 | 27.5 |
| Water | 18.5 | 203.5 |
| Total: | 80.0 | 880.0 (80 mL per sample) |

4. Bring each RNA sample to a total volume of 20 mL in a 1.5 mL microcentrifuge tube (for example, for THP-1 RNA, remove 10 mL RNA and dilute to 20 mL with RNase/DNase free water, for whole blood RNA use 20 mL total RNA) and add 80 mL RT reaction mix from step 5,2,3. Mix by pipetting up and down.

5. Incubate sample at room temperature for 10 minutes.

6. Incubate sample at 37° C. for 1 hour.

7. Incubate sample at 90° C. for 10 minutes.

8. Quick spin samples in microcentrifuge.

9. Place sample on ice if doing PCR immediately, otherwise store sample at −20° C. for future use.

10. PCR QC should be run on all RT samples using 18S and b-actin (see SOP 200-020).

The use of the primer probe with the first strand cDNA as described above to permit measurement of constituents of a Gene Expression Panel is as follows:

Set up of a 24-gene Human Gene Expression Panel for Inflammation.

### Materials

1. 20× Primer/Probe Mix for each gene of interest.

2. 20× Primer/Probe Mix for 18S endogenous control.

3. 2× Taqman Universal PCR Master Mix.

4. cDNA transcribed from RNA extracted from cells.

5. Applied Biosystems 96-Well Optical Reaction Plates.

6. Applied Biosystems Optical Caps, or optical-clear film.

7. Applied Biosystem Prism 7700 Sequence Detector.

### Methods

1. Make stocks of each Primer/Probe mix containing the Primer/Probe for the gene of interest, Primer/Probe for 18S endogenous control, and 2× PCR Master Mix as follows. Make sufficient excess to allow for pipetting error e.g. approximately 10% excess. The following example illustrates a typical set up for one gene with quadruplicate samples testing two conditions (2 plates).

| | 1X(1 well) | 9X (2 plates worth) |
|---|---|---|
| 2X Master Mix | 12.50 | 112.50 |
| 20X 18S Primer/Probe Mix | 1.25 | 11.25 |
| 20X Gene of interest Primer/Probe Mix | 1.25 | 11.25 |
| Total | 15.00 | 135.00 |

2. Make stocks of cDNA targets by diluting 95 μl of cDNA into 2000 μl of water. The amount of cDNA is adjusted to give Ct values between 10 and 18, typically between 12 and 13.

3. Pipette 15 μl of Primer/Probe mix into the appropriate wells of an Applied Biosystems 96-Well Optical Reaction Plate.

4. Pipette 10 μl of cDNA stock solution into each well of the Applied Biosystems 96-Well Optical Reaction Plate.

5. Seal the plate with Applied Biosystems Optical Caps, or optical-clear film.

6. Analyze the plate on the AB Prism 7700 Sequence Detector.

Methods herein may also be applied using proteins where sensitive quantitative techniques, such as an Enzyme Linked ImmunoSorbent Assay (ELISA) or mass spectroscopy, are available and well-known in the art for measuring the amount of a protein constituent. (see WO 98/24935 herein incorporated by reference).

### Baseline Profile Data Sets

The analyses of samples from single individuals and from large groups of individuals provide a library of profile data sets relating to a particular panel or series of panels. These profile data sets may be stored as records in a library for use as baseline profile data sets. As the term “baseline” suggests, the stored baseline profile data sets serve as comparators for providing a calibrated profile data set that is informative about a biological condition or agent. Baseline profile data sets may be stored in libraries and classified in a number of cross-referential ways. One form of classification may rely on the characteristics of the panels from which the data sets are derived. Another form of classification may be by particular biological condition. The concept of biological condition encompasses any state in which a cell or population of cells may be found at any one time. This state may reflect geography of samples, sex of subjects or any other discriminator. Some of the discriminators may overlap. The libraries may also be accessed for records associated with a single subject or particular clinical trial. The classification of baseline profile data sets may further be annotated with medical information about a particular subject, a medical condition, a particular agent etc.

The choice of a baseline profile data set for creating a calibrated profile data set is related to the biological condition to be evaluated, monitored, or predicted, as well as, the intended use of the calibrated panel, e.g., as to monitor drug development, quality control or other uses. It may be desirable to access baseline profile data sets from the same subject for whom a first profile data set is obtained or from different subject at varying times, exposures to stimuli, drugs or complex compounds; or may be derived from like or dissimilar populations or sets of subjects.

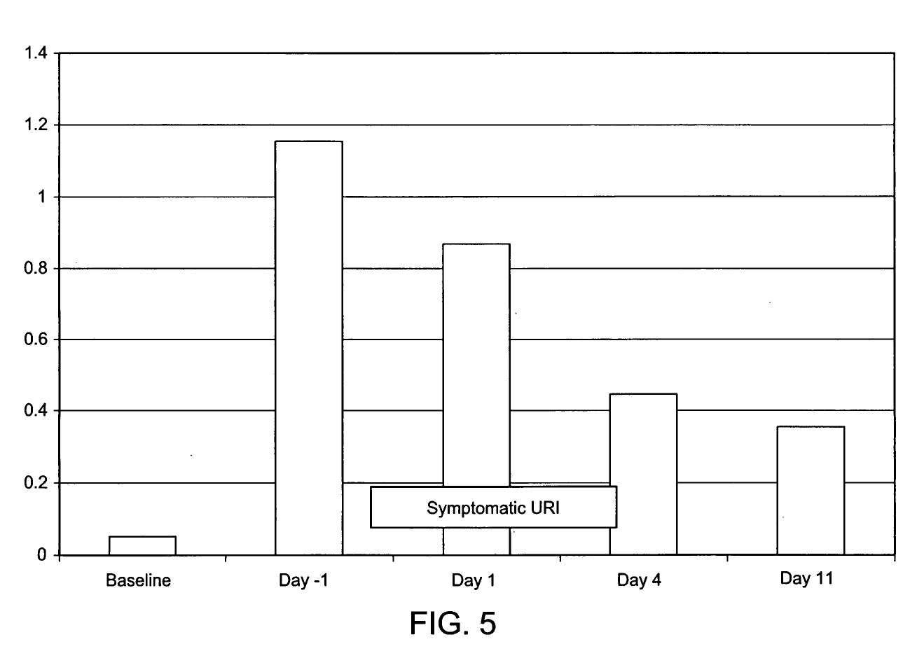

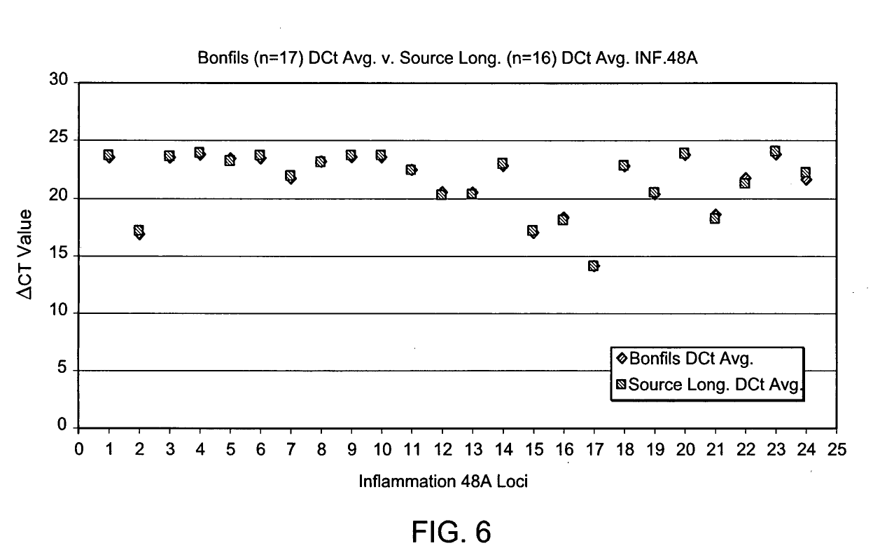

The profile data set may arise from the same subject for which the first data set is obtained, where the sample is taken at a separate or similar time, a different or similar site or in a different or similar biological condition. For example, FIG. 5 provides a protocol in which the sample is taken before stimulation or after stimulation. The profile data set obtained from the unstimulated sample may serve as a baseline profile data set for the sample taken after stimulation. The baseline data set may also be derived from a library containing profile data sets of a population or set of subjects having some defining characteristic or biological condition. The baseline profile data set may also correspond to some ex vivo or in vitro properties associated with an in vitro cell culture. The resultant calibrated profile data sets may then be stored as a record in a database or library (FIG. 6) along with or separate from the baseline profile data base and optionally the first profile data set although the first profile data set would normally become incorporated into a baseline profile data set under suitable classification criteria. The remarkable consistency of Gene Expression Profiles associated with a given biological condition makes it valuable to store profile data, which can be used, among other things for normative reference purposes. The normative reference can serve to indicate the degree to which a subject conforms to a given biological condition (healthy or diseased) and, alternatively or in addition, to provide a target for clinical intervention.

Selected baseline profile data sets may be also be used as a standard by which to judge manufacturing lots in terms of efficacy, toxicity, etc. Where the effect of a therapeutic agent is being measured, the baseline data set may correspond to Gene Expression Profiles taken before administration of the agent. Where quality control for a newly manufactured product is being determined, the baseline data set may correspond with a gold standard for that product. However, any suitable normalization techniques may be employed. For example, an average baseline profile data set is obtained from authentic material of a naturally grown herbal nutriceutical and compared over time and over different lots in order to demonstrate consistency, or lack of consistency, in lots of compounds prepared for release.

### Calibrated Data

Given the repeatability we have achieved in measurement of gene expression, described above in connection with “Gene Expression Panels” and “gene amplification”, we conclude that where differences occur in measurement under such conditions, the differences are attributable to differences in biological condition. Thus we have found that calibrated profile data sets are highly reproducible in samples taken from the same individual under the same conditions. We have similarly found that calibrated profile data sets are reproducible in samples that are repeatedly tested. We have also found repeated instances wherein calibrated profile data sets obtained when samples from a subject are exposed ex vivo to a compound are comparable to calibrated profile data from a sample that has been exposed to a sample in vivo. We have also found, importantly, that an indicator cell line treated with an agent can in many cases provide calibrated profile data sets comparable to those obtained from in vivo or ex vivo populations of cells. Moreover, we have found that administering a sample from a subject onto indicator cells can provide informative calibrated profile data sets with respect to the biological condition of the subject including the health, disease states, therapeutic interventions, aging or exposure to environmental stimuli or toxins of the subject.

### Calculation of Calibrated Profile Data Sets and Computational Aids

The calibrated profile data set may be expressed in a spreadsheet or represented graphically for example, in a bar chart or tabular form but may also be expressed in a three dimensional representation. The function relating the baseline and profile data may be a ratio expressed as a logarithm. The constituent may be itemized on the x-axis and the logarithmic scale may be on the y-axis. Members of a calibrated data set may be expressed as a positive value representing a relative enhancement of gene expression or as a negative value representing a relative reduction in gene expression with respect to the baseline.

Each member of the calibrated profile data set should be reproducible within a range with respect to similar samples taken from the subject under similar conditions. For example, the calibrated profile data sets may be reproducible within one order of magnitude with respect to similar samples taken from the subject under similar conditions. More particularly, the members may be reproducible within 50%, more particularly reproducible within 20%, and typically within 10%. In accordance with embodiments of the invention, a pattern of increasing, decreasing and no change in relative gene expression from each of a plurality of gene loci examined in the Gene Expression Panel may be used to prepare a calibrated profile set that is informative with regards to a biological condition, biological efficacy of an agent treatment conditions or for comparison to populations or sets of subjects or samples, or for comparison to populations of cells. Patterns of this nature may be used to identify likely candidates for a drug trial, used alone or in combination with other clinical indicators to be diagnostic or prognostic with respect to a biological condition or may be used to guide the development of a pharmaceutical or nutriceutical through manufacture, testing and marketing.

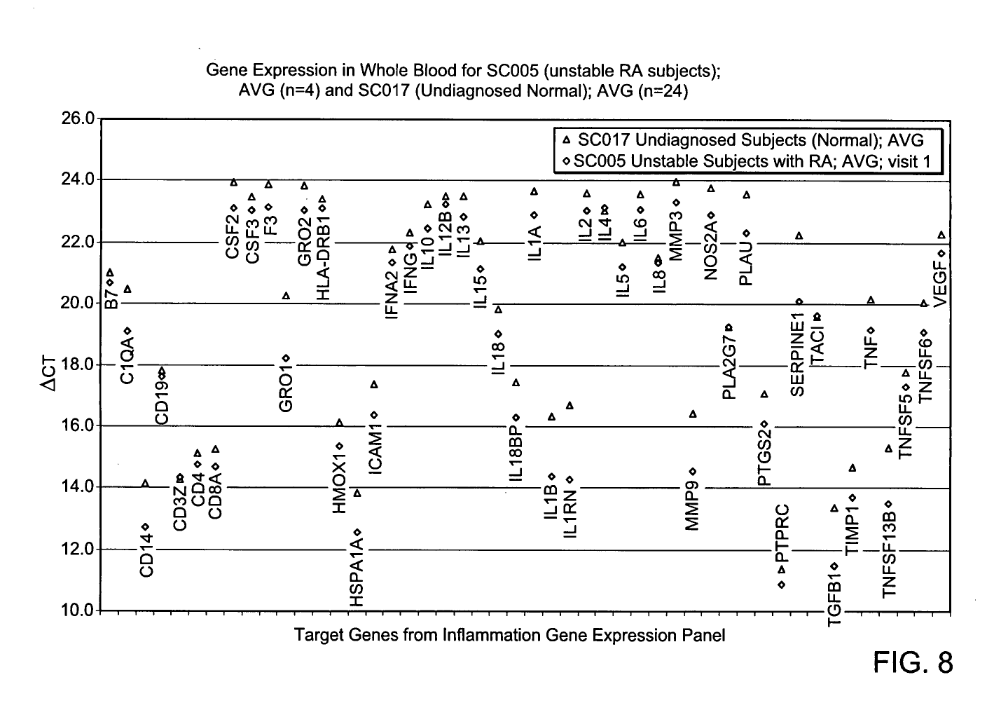

The numerical data obtained from quantitative gene expression and numerical data from calibrated gene expression relative to a baseline profile data set may be stored in databases or digital storage mediums and may retrieved for purposes including managing patient health care or for conducting clinical trials or for characterizing a drug. The data may be transferred in physical or wireless networks via the World Wide Web, email, or internet access site for example or by hard copy so as to be collected and pooled from distant geographic sites (FIG. 8).

In an embodiment of the present invention, a descriptive record is stored in a single database or multiple databases where the stored data includes the raw gene expression data (first profile data set) prior to transformation by use of a baseline profile data set, as well as a record of the baseline profile data set used to generate the calibrated profile data set including for example, annotations regarding whether the baseline profile data set is derived from a particular Signature Panel and any other annotation that facilitates interpretation and use of the data.

Because the data is in a universal format, data handling may readily be done with a computer. The data is organized so as to provide an output optionally corresponding to a graphical representation of a calibrated data set.

For example, a distinct sample derived from a subject being at least one of RNA or protein may be denoted as P_{I}. The first profile data set derived from sample P_{I} is denoted Mⱼ, where Mⱼ is a quantitative measure of a distinct RNA or protein constituent of P_{I}. The record Ri is a ratio of M and P and may be annotated with additional data on the subject relating to, for example, age, diet, ethnicity, gender, geographic location, medical disorder, mental disorder, medication, physical activity, body mass and environmental exposure. Moreover, data handling may further include accessing data from a second condition database which may contain additional medical data not presently held with the calibrated profile data sets. In this context, data access may be via a computer network.

The above described data storage on a computer may provide the information in a form that can be accessed by a user. Accordingly, the user may load the information onto a second access site including downloading the information. However, access may be restricted to users having a password or other security device so as to protect the medical records contained within. A feature of this embodiment of the invention is the ability of a user to add new or annotated records to the data set so the records become part of the biological information.

The graphical representation of calibrated profile data sets pertaining to a product such as a drug provides an opportunity for standardizing a product by means of the calibrated profile, more particularly a signature profile. The profile may be used as a feature with which to demonstrate relative efficacy, differences in mechanisms of actions, etc. compared to other drugs approved for similar or different uses.

The various embodiments of the invention may be also implemented as a computer program product for use with a computer system. The product may include program code for deriving a first profile data set and for producing calibrated profiles. Such implementation may include a series of computer instructions fixed either on a tangible medium, such as a computer readable medium (for example, a diskette, CD-ROM, ROM, or fixed disk), or transmittable to a computer system via a modem or other interface device, such as a communications adapter coupled to a network. The network coupling may be for example, over optical or wired communications lines or via wireless techniques (for example, microwave, infrared or other transmission techniques) or some combination of these. The series of computer instructions preferably embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (for example, shrink wrapped software), preloaded with a computer system (for example, on system ROM or fixed disk), or distributed from a server or electronic bulletin board over a network (for example, the Internet or World Wide Web). In addition, a computer system is further provided including derivative modules for deriving a first data set and a calibration profile data set.

The calibration profile data sets in graphical or tabular form, the associated databases, and the calculated index or derived algorithm, together with information extracted from the panels, the databases, the data sets or the indices or algorithms are commodities that can be sold together or separately for a variety of purposes as described in WO 01/25473.

### Index Construction

In combination, (i) the remarkable consistency of Gene Expression Profiles with respect to a biological condition across a population or set of subject or samples, or across a population of cells and (ii) the use of procedures that provide substantially reproducible measurement of constituents in a Gene Expression Panel giving rise to a Gene Expression Profile, under measurement conditions wherein specificity and efficiencies of amplification for all constituents of the panel are substantially similar, make possible the use of an index that characterizes a Gene Expression Profile, and which therefore provides a measurement of a biological condition.

An index may be constructed using an index function that maps values in a Gene Expression Profile into a single value that is pertinent to the biological condition at hand. The values in a Gene Expression Profile are the amounts of each constituent of the Gene Expression Panel that corresponds to the Gene Expression Profile. These constituent amounts form a profile data set, and the index function generates a single value—the index—from the members of the profile data set.

The index function may conveniently be constructed as a linear sum of terms, each term being what we call a “contribution function” of a member of the profile data set. For example, the contribution function may be a constant times a power of a member of the profile data set. So the index function would have the form
I=ΣCᵢMᵢ^{P(i)},
where I is the index, Mᵢ is the value of the member i of the profile data set, Cᵢ is a constant, and P(i) is a power to which Mᵢ is raised, the sum being formed for all integral values of i up to the number of members in the data set. We thus have a linear polynomial expression.

The values Cᵢ and P(i) may be determined in a number of ways, so that the index I is informative of the pertinent biological condition. One way is to apply statistical techniques, such as latent class modeling, to the profile data sets to correlate clinical data or experimentally derived data, or other data pertinent to the biological condition. In this connection, for example, may be employed the software from Statistical Innovations, Belmont, Mass., called Latent Gold®. See the web pages at statisticalinnovations.com/lg/, which are hereby incorporated herein by reference.

Alternatively, other simpler modeling techniques may be employed in a manner known in the art. The index function for inflammation may be constructed, for example, in a manner that a greater degree of inflammation (as determined by the a profile data set for the Inflammation Gene Expression Profile) correlates with a large value of the index function. In a simple embodiment, therefore, each P(i) may be +1 or −1, depending on whether the constituent increases or decreases with increasing inflammation. As discussed in further detail below, we have constructed a meaningful inflammation index that is proportional to the expression
¼{IL1A}+¼{IL1B}+¼{TNF}+¼{INFG}−1/{IL10},
where the braces around a constituent designate measurement of such constituent and the constituents are a subset of the Inflammation Gene Expression Panel of Table 1.

Just as a baseline profile data set, discussed above, can be used to provide an appropriate normative reference, and can even be used to create a Calibrated profile data set, as discussed above, based on the normative reference, an index that characterizes a Gene Expression Profile can also be provided with a normative value of the index function used to create the index. This normative value can be determined with respect to a relevant population or set of subjects or samples or to a relevant population of cells, so that the index may be interpreted in relation to the normative value. The relevant population or set of subjects or samples, or relevant population of cells may have in common a property that is at least one of age range, gender, ethnicity, geographic location, nutritional history, medical condition, clinical indicator, medication, physical activity, body mass, and environmental exposure.

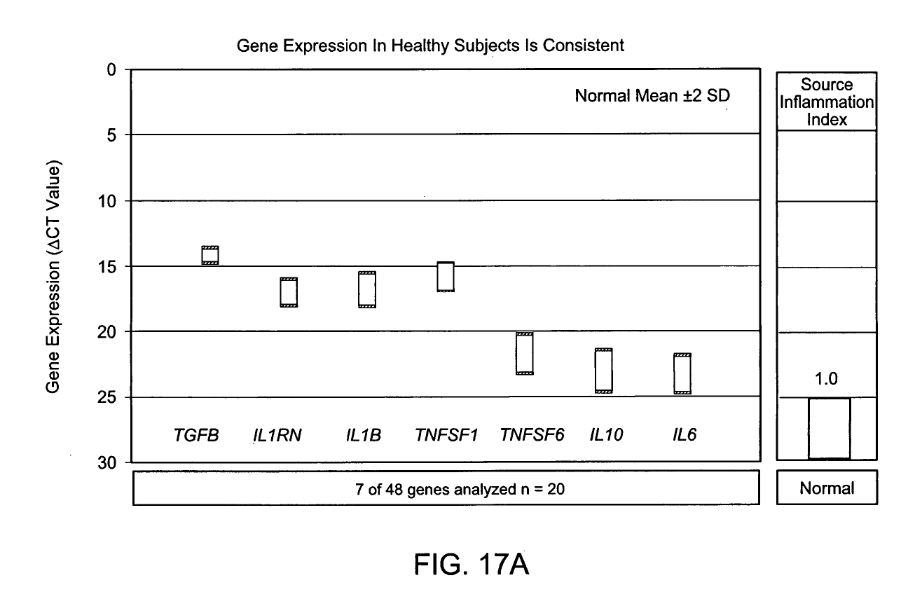

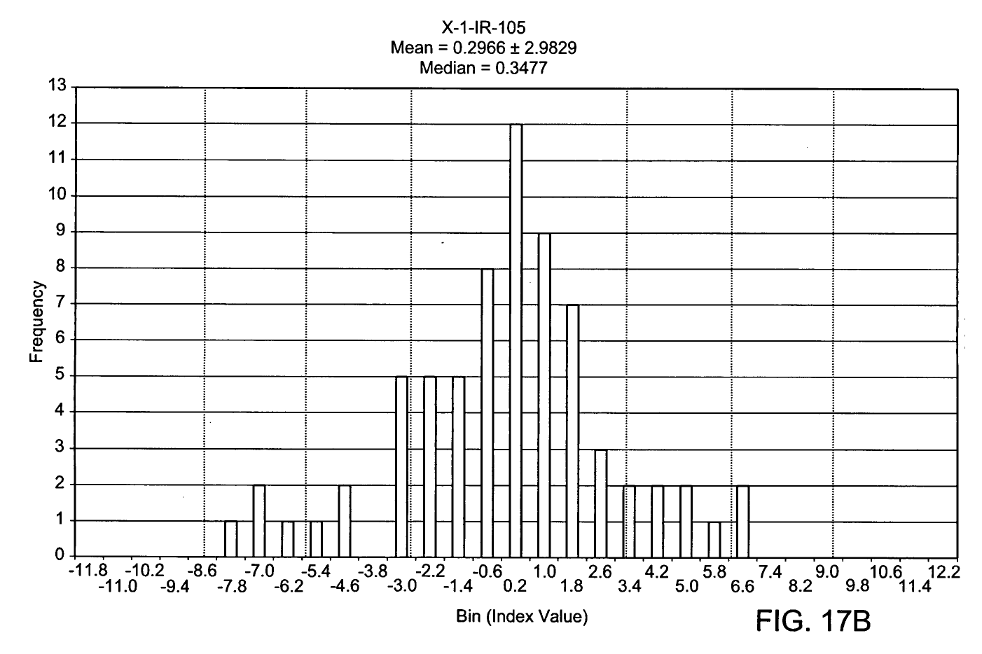

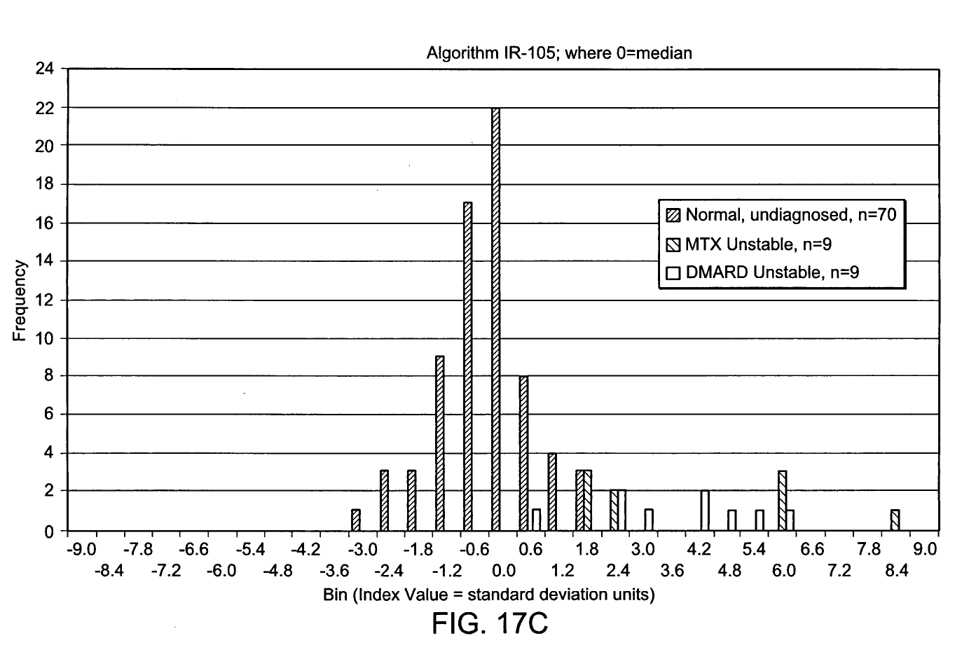

As an example, the index can be constructed, in relation to a normative Gene Expression Profile for a population or set of healthy subjects, in such a way that a reading of approximately 1 characterizes normative Gene Expression Profiles of healthy subjects. Let us further assume that the biological condition that is the subject of the index is inflammation; a reading of 1 in this example thus corresponds to a Gene Expression Profile that matches the norm for healthy subjects. A substantially higher reading then may identify a subject experiencing an inflammatory condition. The use of 1 as identifying a normative value, however, is only one possible choice; another logical choice is to use 0 as identifying the normative value. With this choice, deviations in the index from zero can be indicated in standard deviation units (so that values lying between −1 and +1 encompass 90% of a normally distributed reference population or set of subjects. Since we have found that Gene Expression Profile values (and accordingly constructed indices based on them) tend to be normally distributed, the 0-centered index constructed in this manner is highly informative. It therefore facilitates use of the index-in diagnosis of disease and setting objectives for treatment. The choice of 0 for the normative value, and the use of standard deviation units, for example, are illustrated in FIG. 17B, discussed below.

### EXAMPLES

### Example 1

### Acute Inflammatory Index to Assist in Analysis of Large, Complex Data Sets.

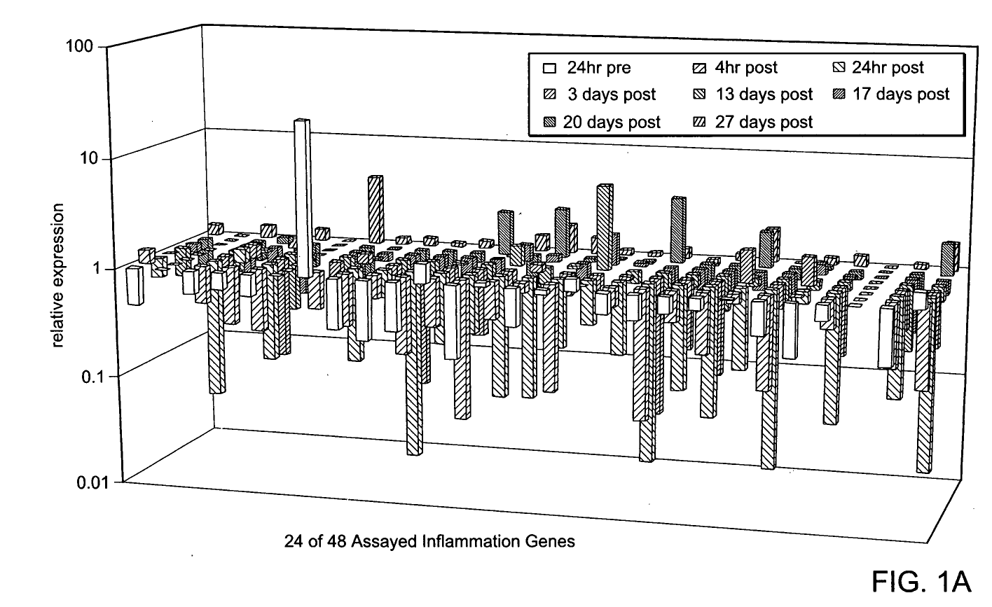

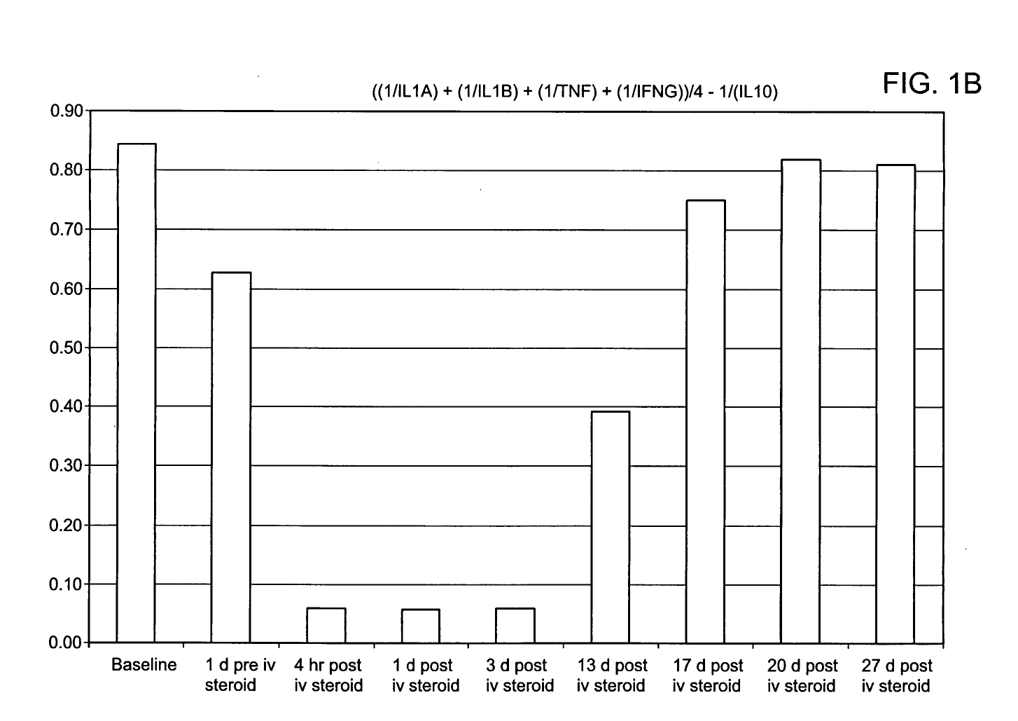

In one embodiment of the invention the index value or algorithm can be used to reduce a complex data set to a single index value that is informative with respect to the inflammatory state of a subject. This is illustrated in FIGS. 1A and 1B.

FIG. 1A is entitled Source Precision Inflammation Profile Tracking of A Subject Results in a Large, Complex Data Set. The figure shows the results of assaying 24 genes from the Inflammation Gene Expression Panel (shown in Table 1) on eight separate days during the course of optic neuritis in a single male subject.

FIG. 1B shows use of an Acute Inflammation Index. The data displayed in FIG. 1A above is shown in this figure after calculation using an index function proportional to the following mathematical expression: (¼{IL1A}+¼{IL1B}+¼{TNF}+¼{INFG}−1/{IL10}).

### Example 2

### Use of Acute Inflammation Index or Algorithm to Monitor a Biological Condition of a Sample or a Subject

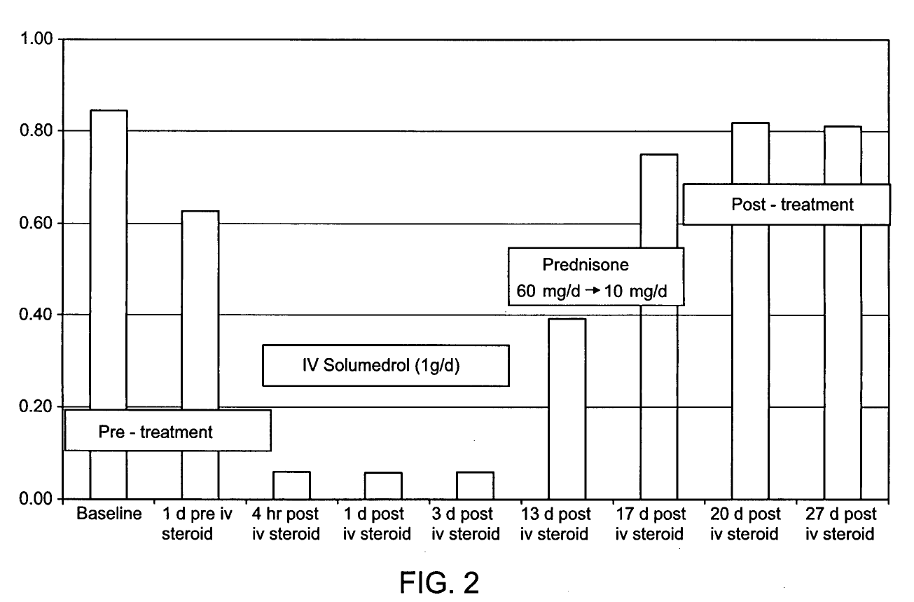

The inflammatory state of a subject reveals information about the past progress of the biological condition, future progress, response to treatment, etc. The Acute Inflammation Index may be used to reveal such information about the biological condition of a subject. This is illustrated in FIG. 2.

The results of the assay for inflammatory gene expression for each day (shown for 24 genes in each row of FIG. 1A) is displayed as an individual histogram after calculation. The index reveals clear trends in inflammatory status that may correlated with therapeutic intervention (FIG. 2).

FIG. 2 is a graphical illustration of the acute inflammation index calculated at 9 different, significant clinical milestones from blood obtained from a single patient treated medically with for optic neuritis. Changes in the index values for the Acute Inflammation Index correlate strongly with the expected effects of therapeutic intervention. Four clinical milestones have been identified on top of the Acute Inflammation Index in this figure including (1) prior to treatment with steroids, (2) treatment with IV solumedrol at 1 gram per day, (3) post-treatment with oral prednisone at 60 mg per day tapered to 10 mg per day and (4) post treatment. The data set is the same as for FIG. 1. The index is proportional to ¼{IL1A}+¼{]ILB}+¼{TNF}+¼{INFG}−1/{IL10}. As expected, the acute inflammation index falls rapidly with treatment with IV steroid, goes up during less efficacious treatment with oral prednisone and returns to the pre-treatment level after the steroids have been discontinued and metabolized completely.

### Example 3

### Use of the Acute Inflammatory Index to Set Dose

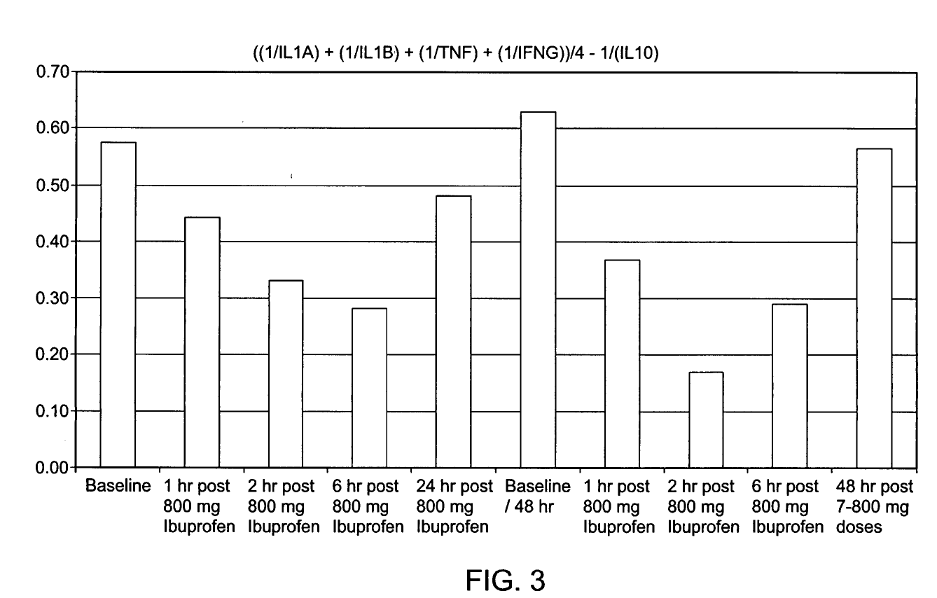

Including concentrations and timing, for compounds in development or for compounds to be tested in human and non-human subjects as shown in FIG. 3. The acute inflammation index may be used as a common reference value for therapeutic compounds or interventions without common mechanisms of action. The compound that induces a gene response to a compound as indicated by the index, but fails to ameliorate a known biological conditions may be compared to a different compounds with varying effectiveness in treating the biological condition.

FIG. 3 shows the effects of single dose treatment with 800 mg of ibuprofen in a single donor as characterized by the Acute Inflammation Index. 800 mg of over-the-counter ibuprofen were taken by a single subject at Time=0 and Time=48 hr. Gene expression values for the indicated five inflammation-related gene loci were determined as described below at times=2, 4, 6, 48, 50, 56 and 96 hours. As expected the acute inflammation index falls immediately after taking the non-steroidal anti-inflammatory ibuprofen and returns to baseline after 48 hours. A second dose at T=48 follows the same kinetics at the first dose and returns to baseline at the end of the experiment at T=96.

### Example 4

### Use of the Acute Inflammation Index to Characterize Efficacy, Safety, and Mode of Physiological Action for an Agent

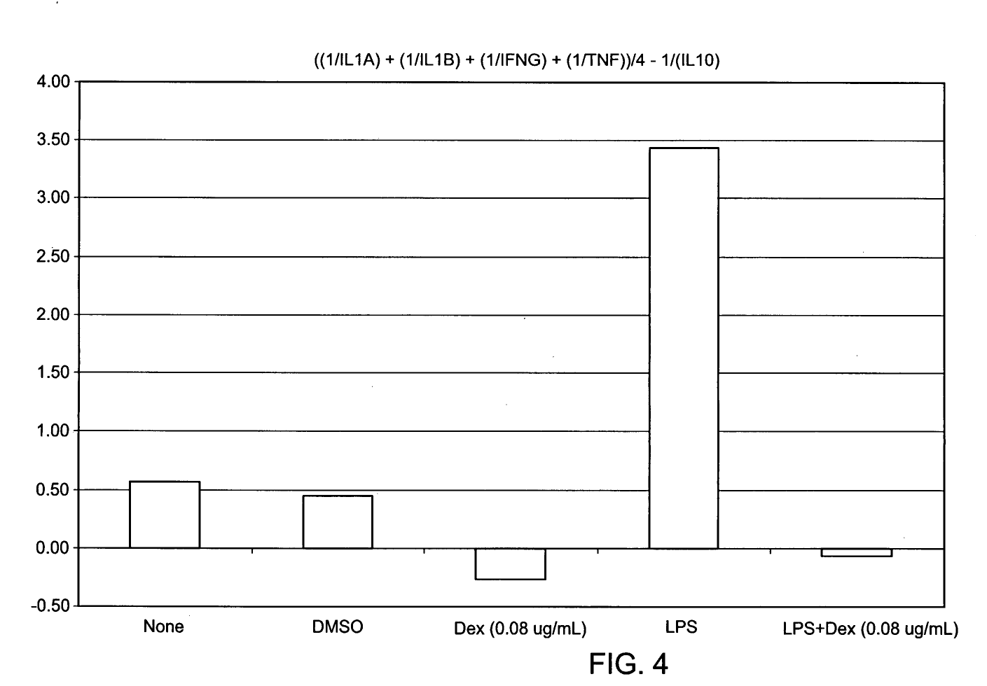

Which may be in development and/or may be complex in nature. This is illustrated in FIG. 4.

FIG. 4 shows that the calculated acute inflammation index displayed graphically for five different conditions including (A) untreated whole blood; (B) whole blood treated in vitro with DMSO, an non-active carrier compound; (C) otherwise unstimulated whole blood treated in vitro with dexamethasone (0.08 ug/ml); (D) whole blood stimulated in vitro with lipopolysaccharide, a known pro-inflammatory compound, (LPS, 1 ng/ml) and (E) whole blood treated in vitro with LPS (1 ng/ml) and dexamethasone (0.08 ug/ml). Dexamethasone is used as a prescription compound that is commonly used medically as an anti-inflammatory steroid compound. The acute inflammation index is calculated from the experimentally determined gene expression levels of inflammation-related genes expressed in human whole blood obtained from a single patient. Results of mRNA expression are expressed as Ct's in this example, but may be expressed as, e.g., relative fluorescence units, copy number or any other quantifiable, precise and calibrated form, for the genes IL1A, IL1B, TNF, IFNG and IL10. From the gene expression values, the acute inflammation values were determined algebraically according in proportion to the expression ¼{IL1A}+¼{IL1B}+¼{TNF}+¼{INFG}−1/{IL10}.

### Example 5

### Development and Use of Population Normative Values for Gene Expression Profiles

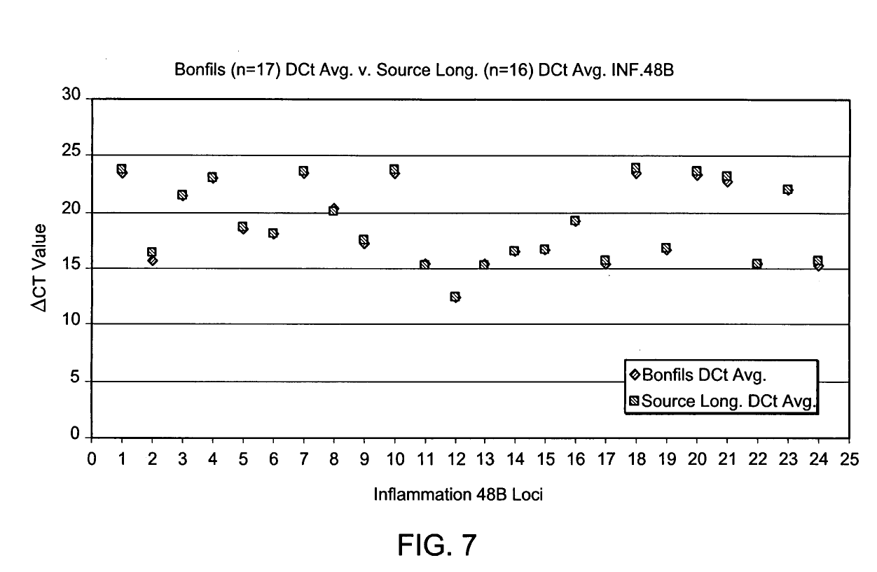

FIGS. 6 and 7 show the arithmetic mean values for gene expression profiles (using the 48 loci of the Inflammation Gene Expression Panel of Table 1) obtained from whole blood of two distinct patient populations (patient sets). These patient sets are both normal or undiagnosed. The first patient set, which is identified as Bonfils (the plot points for which are represented by diamonds), is composed of 17 subjects accepted as blood donors at the Bonfils Blood Center in Denver, Colo. The second patient set is 9 donors, for which Gene Expression Profiles were obtained from assays conducted four times over a four-week period. Subjects in this second patient set (plot points for which are represented by squares) were recruited from employees of Source Precision Medicine, Inc., the assignee herein. Gene expression averages for each population were calculated for each of 48 gene loci of the Gene Expression Inflammation Panel. The results for loci 1-24 (sometimes referred to below as the Inflammation 48A loci) are shown in FIG. 6 and for loci 25-48 (sometimes referred to below as the Inflammation 48B loci) are shown in FIG. 7.

The consistency between gene expression levels of the two distinct patient sets is dramatic. Both patient sets show gene expressions for each of the 48 loci that are not significantly different from each other. This observation suggests that there is a “normal” expression pattern for human inflammatory genes, that a Gene Expression Profile, using the Inflammation Gene Expression Panel of Table 1 (or a subset thereof) characterizes that expression pattern, and that a population-normal expression pattern can be used, for example, to guide medical intervention for any biological condition that results in a change from the normal expression pattern.

In a similar vein, FIG. 8 shows arithmetic mean values for gene expression profiles (again using the 48 loci of the Inflammation Gene Expression Panel of Table 1) also obtained from whole blood of two distinct patient populations (patient sets). One patient set, expression values for which are represented by triangular data points, is 24 normal, undiagnosed subjects (who therefore have no known inflammatory disease). The other patient set, the expression values for which are represented by diamond-shaped data points, is four patients with rheumatoid arthritis and who have failed therapy (who therefore have unstable rheumatoid arthritis).

As remarkable as the consistency of data from the two distinct normal patient sets shown in FIGS. 6 and 7 is the systematic divergence of data from the normal and diseased patient sets shown in FIG. 8. In 45 of the shown 48 inflammatory gene loci, subjects with unstable rheumatoid arthritis showed, on average, increased inflammatory gene expression (lower cycle threshold values; Ct), than subjects without disease. The data thus further demonstrate that is possible to identify groups with specific biological conditions using gene expression if the precision and calibration of the underlying assay are carefully designed and controlled according to the teachings herein.

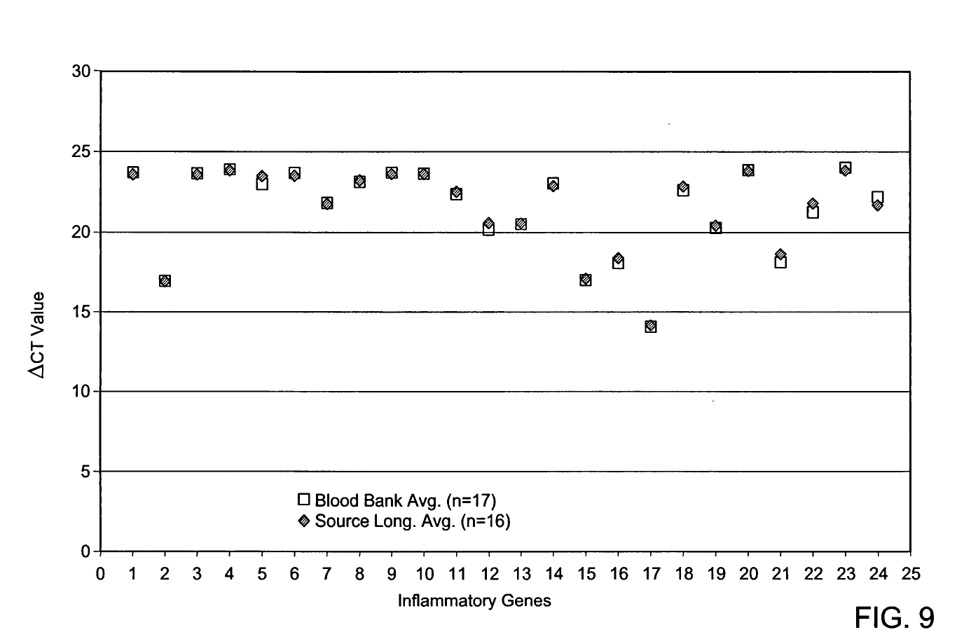

FIG. 9, in a manner analogous to FIG. 8, shows the shows arithmetic mean values for gene expression profiles using 24 loci of the Inflammation Gene Expression Panel of Table 1) also obtained from whole blood of two distinct patient sets. One patient set, expression values for which are represented by diamond-shaped data points, is 17 normal, undiagnosed subjects (who therefore have no known inflammatory disease) who are blood donors. The other patient set, the expression values for which are represented by square-shaped data points, is 16 subjects, also normal and undiagnosed, who have been monitored over six months, and the averages of these expression values are represented by the square-shaped data points. Thus the cross-sectional gene expression-value averages of a first healthy population match closely the longitudinal gene expression-value averages of a second healthy population, with approximately 7% or less variation in measured expression value on a gene-to-gene basis.

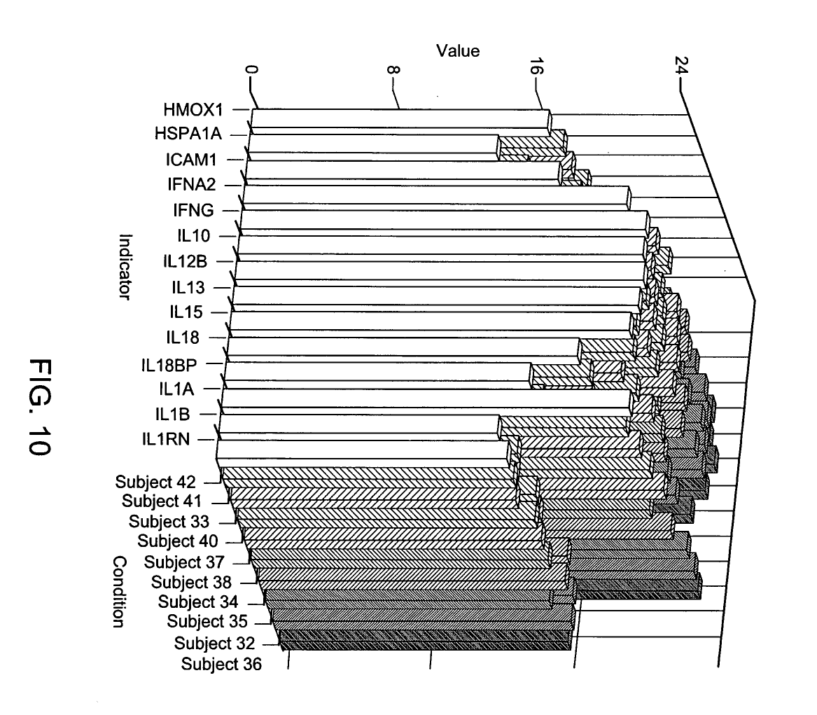

FIG. 10 shows the shows gene expression values (using 14 loci of the Inflammation Gene Expression Panel of Table 1) obtained from whole blood of 44 normal undiagnosed blood donors (data for 10 subjects of which is shown). Again, the gene expression values for each member of the population (set) are closely matched to those for the entire set, represented visually by the consistent peak heights for each of the gene loci. Other subjects of the set and other gene loci than those depicted here display results that are consistent with those shown here.

In consequence of these principles, and in various embodiments of the present invention, population normative values for a Gene Expression Profile can be used in comparative assessment of individual subjects as to biological condition, including both for purposes of health and/or disease. In one embodiment the normative values for a Gene Expression Profile may be used as a baseline in computing a “calibrated profile data set” (as defined at the beginning of this section) for a subject that reveals the deviation of such subject's gene expression from population normative values. Population normative values for a Gene Expression Profile can also be used as baseline values in constructing index functions in accordance with embodiments of the present invention. As a result, for example, an index function can be constructed to reveal not only the extent of an individual's inflammation expression generally but also in relation to normative values.

### Example 6

### Consistency of Expression Values, of Constituents in Gene Expression Panels, Over Time as Reliable Indicators of Biological Condition

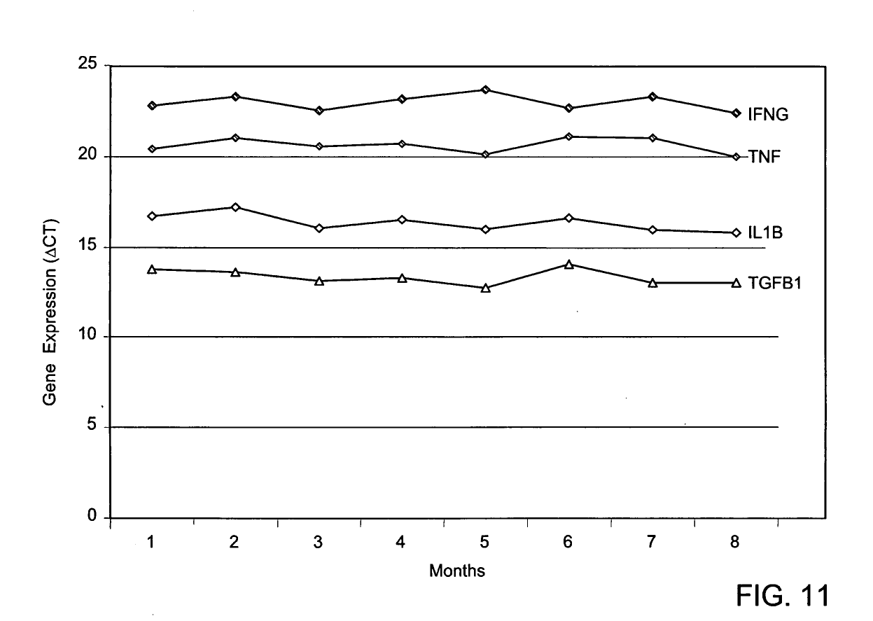

FIG. 11 shows the expression levels for each of four genes (of the Inflammation Gene Expression Panel of Table 1), of a single subject, assayed monthly over a period of eight months. It can be seen that the expression levels are remarkably consistent over time.

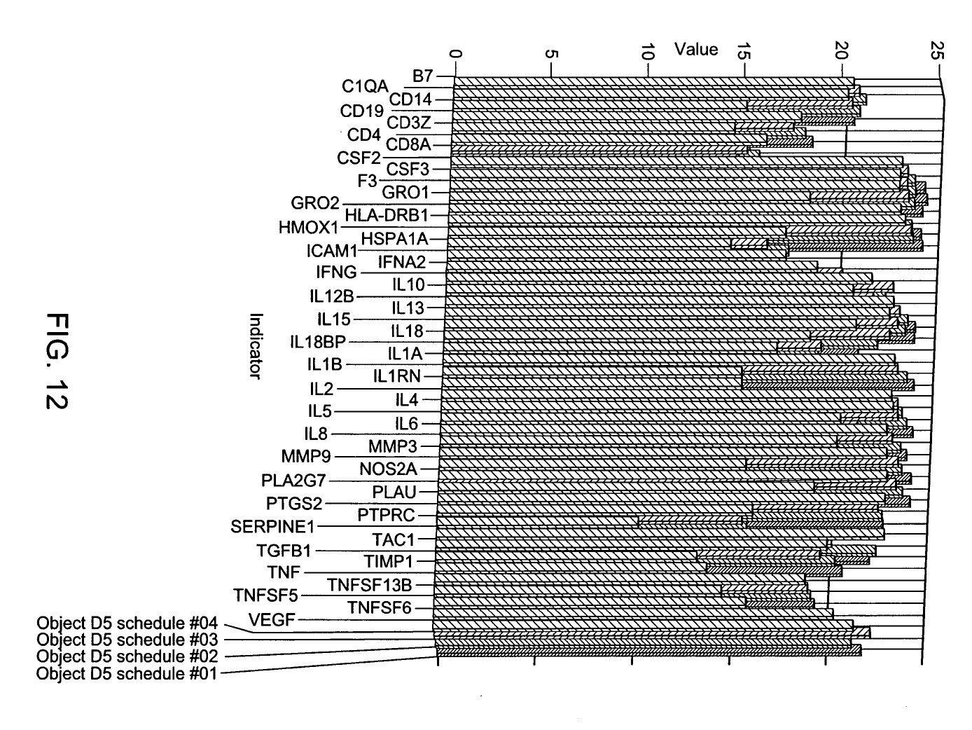

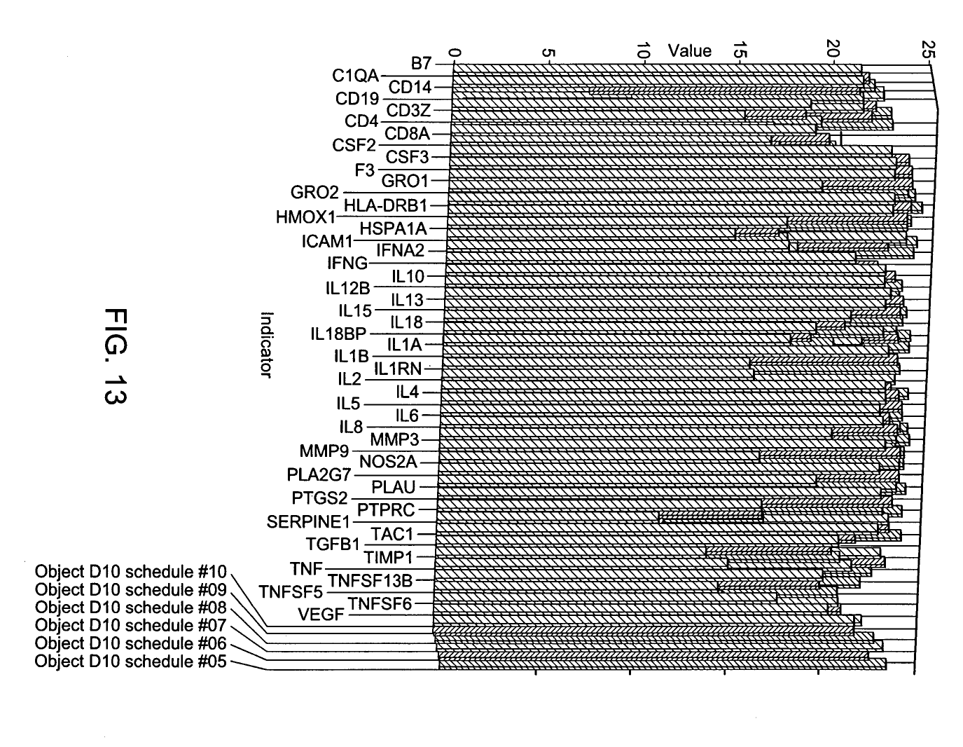

FIGS. 12 and 13 similarly show in each case the expression levels for each of 48 genes (of the Inflammation Gene Expression Panel of Table 1), of distinct single subjects (selected in each case on the basis of feeling well and not taking drugs), assayed, in the case of FIG. 12 weekly over a period of four weeks, and in the case of FIG. 13 monthly over a period of six months. In each case, again the expression levels are remarkably consistent over time, and also similar across individuals.

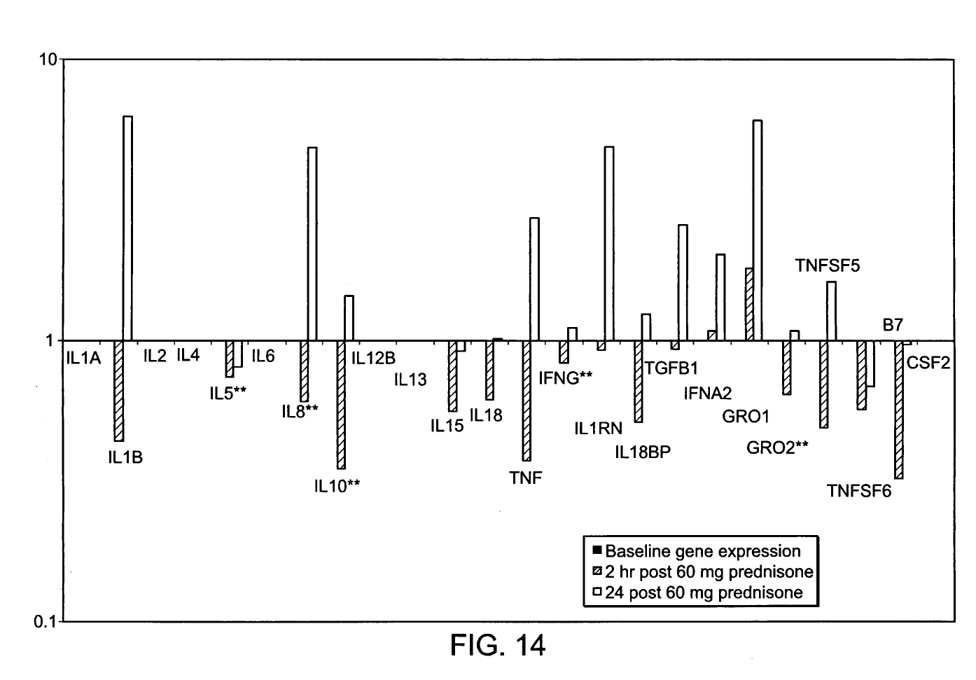

FIG. 14 also shows the effect over time, on inflammatory gene expression in a single human subject, of the administration of an anti-inflammatory steroid, as assayed using the Inflammation Gene Expression Panel of Table 1. In this case, 24 of 48 loci are displayed. The subject had a baseline blood sample drawn in a PAX RNA isolation tube and then took a single 60 mg dose of prednisone, an anti-inflammatory, prescription steroid. Additional blood samples were drawn at 2 hr and 24 hr post the single oral dose. Results for gene expression are displayed for all three time points, wherein values for the baseline sample are shown as unity on the x-axis. As expected, oral treatment with prednisone resulted in the decreased expression of most of inflammation-related gene loci, as shown by the 2-hour post-administration bar graphs. However, the 24-hour post-administration bar graphs show that, for most of the gene loci having reduced gene expression at 2 hours, there were elevated gene expression levels at 24 hr.

Although the baseline in FIG. 14 is based on the gene expression values before drug intervention associated with the single individual tested, we know from the previous example, that healthy individuals tend toward population normative values in a Gene Expression Profile using the Inflammation Gene Expression Panel of Table 1 (or a subset of it). We conclude from FIG. 14 that in an attempt to return the inflammatory gene expression levels to those demonstrated in FIGS. 6 and 7 (normal or set levels), interference with the normal expression induced a compensatory gene expression response that over-compensated for the drug-induced response, perhaps because the prednisone had been significantly metabolized to inactive forms or eliminated from the subject.

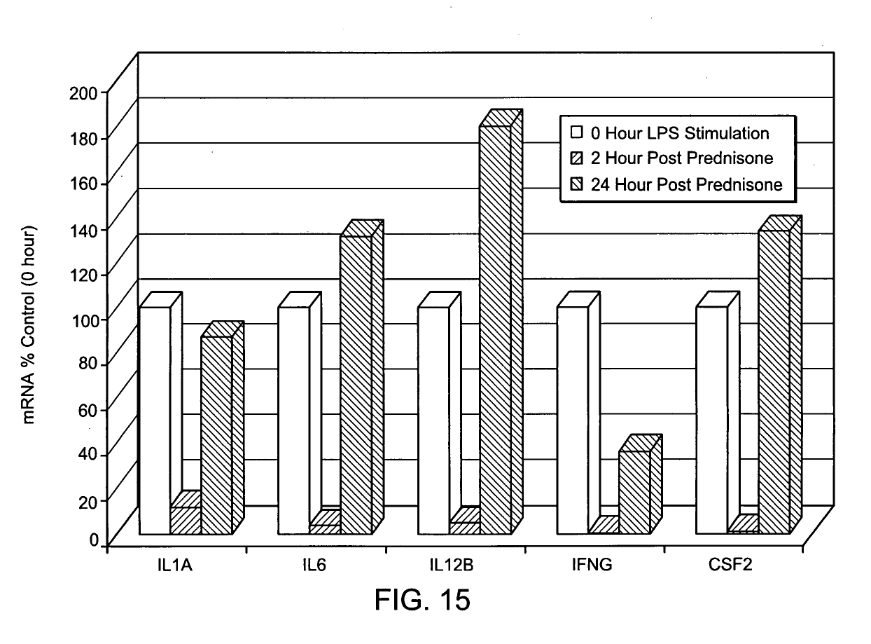

FIG. 15, in a manner analogous to FIG. 14, shows the effect over time, via whole blood samples obtained from a human subject, administered a single dose of prednisone, on expression of 5 genes (of the Inflammation Gene Expression Panel of Table 1). The samples were taken at the time of administration (t=0) of the prednisone, then at two and 24 hours after such administration. Each whole blood sample was challenged by the addition of 0.1 ng/ml of lipopolysaccharide (a Gram-negative endotoxin) and a gene expression profile of the sample, post-challenge, was determined. It can seen that the two-hour sample shows dramatically reduced gene expression of the 5 loci of the Inflammation Gene Expression Panel, in relation to the expression levels at the time of administration (t=0). At 24 hours post administration, the inhibitory effect of the prednisone is no longer apparent, and at 3 of the 5 loci, gene expression is in fact higher than at t=0, illustrating quantitatively at the molecular level the well-known rebound effect.

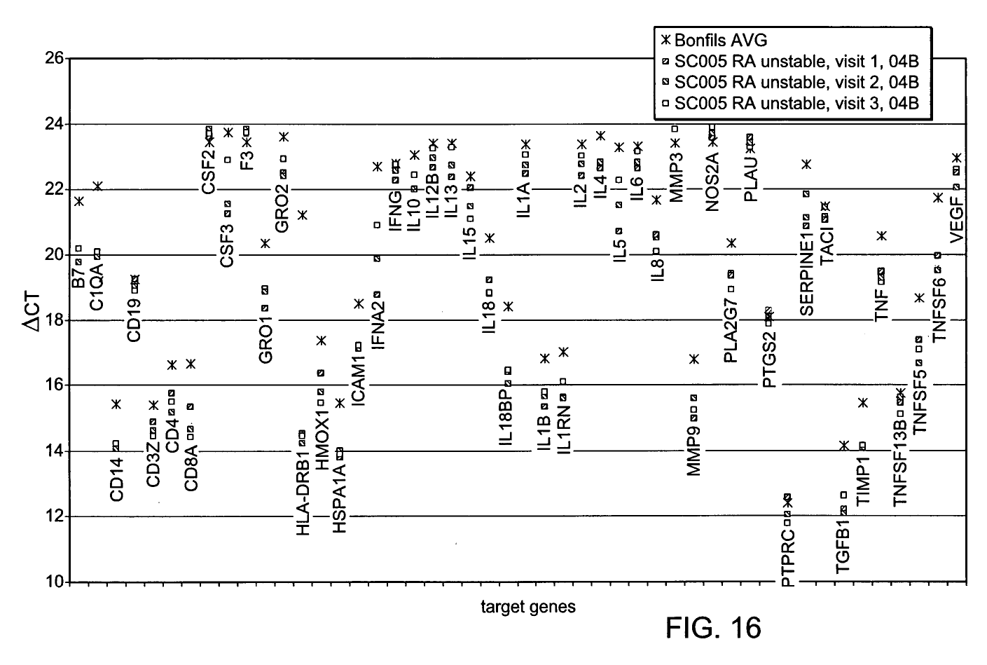

FIG. 16 also shows the effect over time, on inflammatory gene expression in a single human subject suffering from rheumatoid arthritis, of the administration of a TNF-inhibiting compound, but here the expression is shown in comparison to the cognate locus average previously determined (in connection with FIGS. 6 and 7) for the normal (i.e., undiagnosed, healthy) patient set. As part of a larger international study involving patients with rheumatoid arthritis, the subject was followed over a twelve-week period. The subject was enrolled in the study because of a failure to respond to conservative drug therapy for rheumatoid arthritis and a plan to change therapy and begin immediate treatment with a TNF-inhibiting compound. Blood was drawn from the subject prior to initiation of new therapy (visit 1). After initiation of new therapy, blood was drawn at 4 weeks post change in therapy (visit 2), 8 weeks (visit 3), and 12 weeks (visit 4) following the start of new therapy. Blood was collected in PAX RNA isolation tubes, held at room temperature for two hours and then frozen at −30° C.

Frozen samples were shipped to the central laboratory at Source Precision Medicine, the assignee herein, in Boulder, Colo. for determination of expression levels of genes in the 48-gene Inflammation Gene Expression Panel of Table 1. The blood samples were thawed and RNA extracted according to the manufacturer's recommended procedure. RNA was converted to cDNA and the level of expression of the 48 inflammatory genes was determined. Expression results are shown for 11 of the 48 loci in FIG. 16. When the expression results for the 11 loci are compared from visit one to a population average of normal blood donors from the United States, the subject shows considerable difference. Similarly, gene expression levels at each of the subsequent physician visits for each locus are compared to the same normal average value. Data from visits 2, 3 and 4 document the effect of the change in therapy. In each visit following the change in the therapy, the level of inflammatory gene expression for 10 of the 11 loci is closer to the cognate locus -average previously determined for the normal (i.e., undiagnosed, healthy) patient set.

FIG. 17A further illustrates the consistency of inflammatory gene expression, illustrated here with respect to 7 loci of (of the Inflammation Gene Expression Panel of Table 1), in a set of 44 normal, undiagnosed blood donors. For each individual locus is shown the range of values lying within ±2 standard deviations of the mean expression value, which corresponds to 95% of a normally distributed population. Notwithstanding the great width of the confidence interval (95%), the measured gene expression value (ΔCT)—remarkably—still lies within 10% of the mean, regardless of the expression level involved. As described in further detail below, for a given biological condition an index can be constructed to provide a measurement of the condition. This is possible as a result of the conjunction of two circumstances: (i) there is a remarkable consistency of Gene Expression Profiles with respect to a biological condition across a population and (ii) there can be employed procedures that provide substantially reproducible measurement of constituents in a Gene Expression Panel giving rise to a Gene Expression Profile, under measurement conditions wherein specificity and efficiencies of amplification for all constituents of the panel are substantially similar and which therefore provides a measurement of a biological condition. Accordingly, a function of the expression values of representative constituent loci of FIG. 17A is here used to generate an inflammation index value, which is normalized so that a reading of 1 corresponds to constituent expression values of healthy subjects, as shown in the right-hand portion of FIG. 17A.

In FIG. 17B, an inflammation index value was determined for each member of a set of 42 normal undiagnosed blood donors, and the resulting distribution of index values, shown in the figure, can be seen to approximate closely a normal distribution, notwithstanding the relatively small subject set size. The values of the index are shown relative to a 0-based median, with deviations from the median calibrated in standard deviation units. Thus 90% of the subject set lies within +1 and −1 of a 0 value. We have constructed various indices, which exhibit similar behavior.

FIG. 17C illustrates the use of the same index as FIG. 17B, where the inflammation median for a normal population of subjects has been set to zero and both normal and diseased subjects are plotted in standard deviation units relative to that median. An inflammation index value was determined for each member of a normal, undiagnosed population of 70 individuals (black bars). The resulting distribution of index values, shown in FIG. 17C, can be seen to approximate closely a normal distribution. Similarly, index values were calculated for individuals from two diseased population groups, (1) rheumatoid arthritis patients treated with methotrexate (MTX) who are about to change therapy to more efficacious drugs (e.g., TNF inhibitors)(hatched bars), and (2) rheumatoid arthritis patients treated with disease modifying anti-rheumatoid drugs (DMARDS) other than MTX, who are about to change therapy to more efficacious drugs (e.g., MTX). Both populations of subjects present index values that are skewed upward (demonstrating increased inflammation) in comparison to the normal distribution. This figure thus illustrates the utility of an index to derived from Gene Expression Profile data to evaluate disease status and to provide an objective and quantifiable treatment objective. When these two populations of subjects were treated appropriately, index values from both populations returned to a more normal distribution (data not shown here).

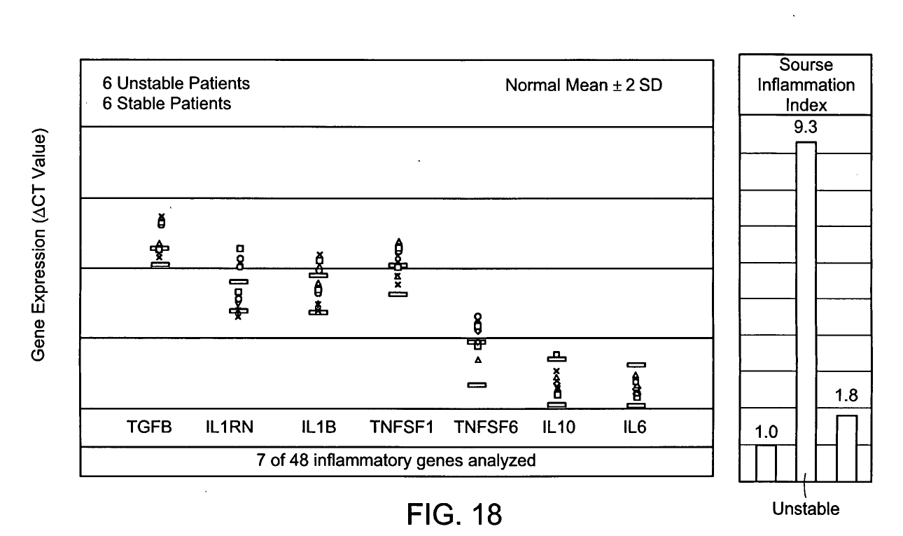

FIG. 18 plots, in a fashion similar to that of FIG. 17A, Gene Expression Profiles, for the same 7 loci as in FIG. 17A, two different 6-subject populations of rheumatoid arthritis patients. One population (called “stable” in the figure) is of patients who have responded well to treatment and the other population (called “unstable” in the figure) is of patients who have not responded well to treatment and whose therapy is scheduled for change. It can be seen that the expression values for the stable patient population, lie within the range of the 95% confidence interval, whereas the expression values for the unstable patient population for 5 of the 7 loci are outside and above this range. The right-hand portion of the figure shows an average inflammation index of 9.3 for the unstable population and an average inflammation index of 1.8 for the stable population, compared to 1 for a normal undiagnosed population of patients. The index thus provides a measure of the extent of the underlying inflammatory condition, in this case, rheumatoid arthritis. Hence the index, besides providing a measure of biological condition, can be used to measure the effectiveness of therapy as well as to provide a target for therapeutic intervention.

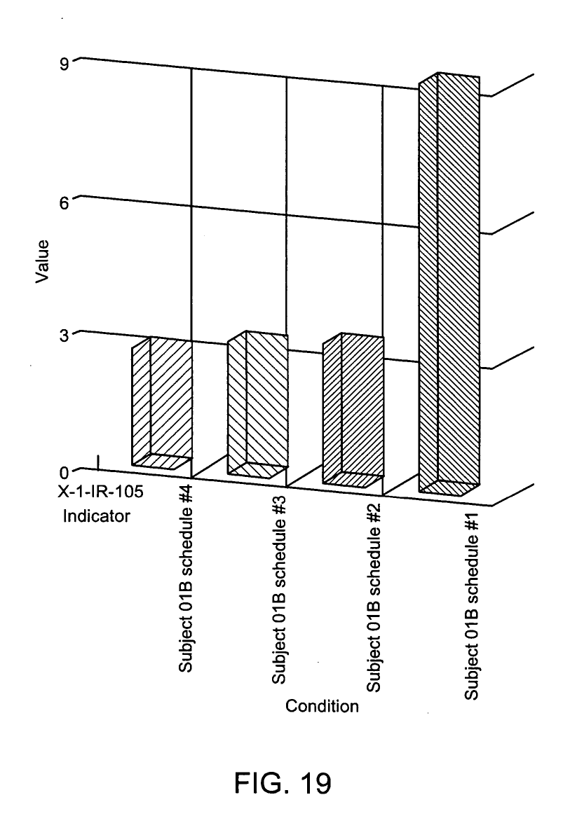

FIG. 19 thus illustrates use of the inflammation index for assessment of a single subject suffering from rheumatoid arthritis, who has not responded well to traditional therapy with methotrexate. The inflammation index for this subject is shown on the far right at start of a new therapy (a TNF inhibitor), and then, moving leftward, successively, 2 weeks, 6 weeks, and 12 weeks thereafter. The index can be seen moving towards normal, consistent with physician observation of-the patient as responding to the new treatment.

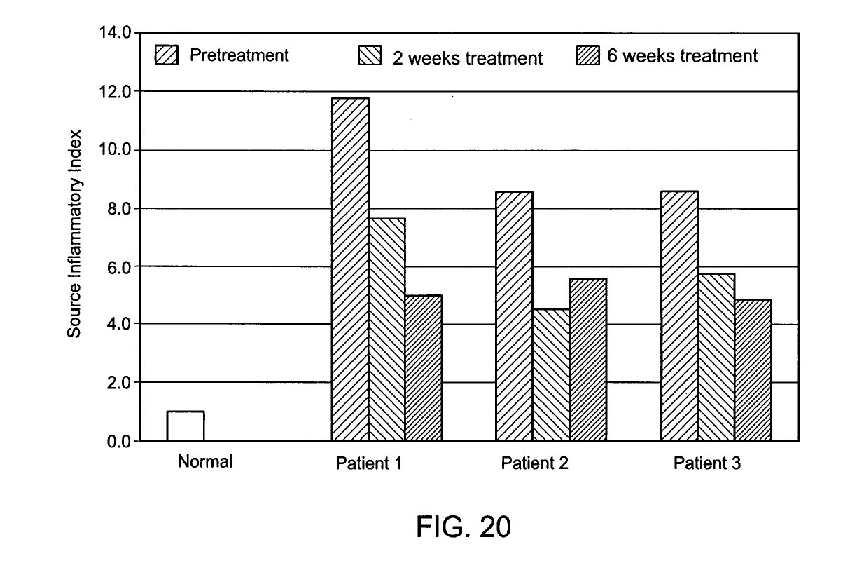

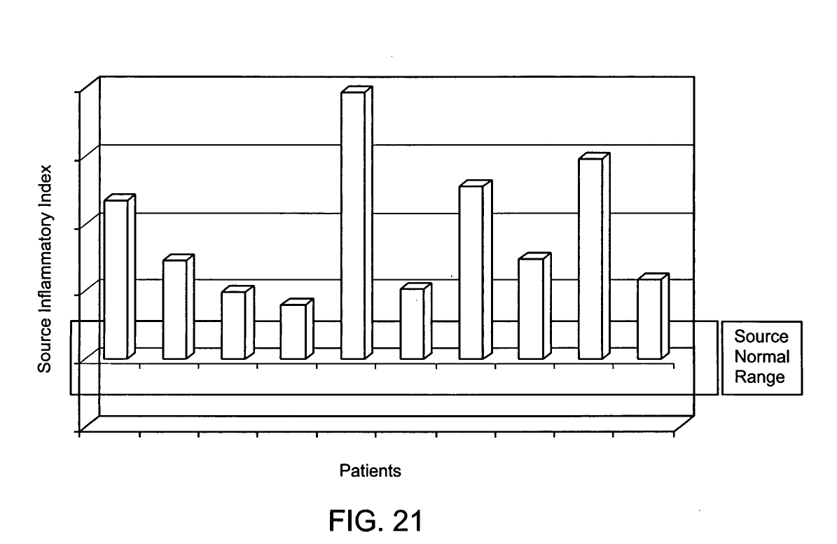

FIG. 20 similarly illustrates use of the inflammation index for assessment of three subjects suffering from rheumatoid arthritis, who have not responded well to traditional therapy with methotrexate, at the beginning of new treatment (also with a TNF inhibitor), and 2 weeks and 6 weeks thereafter. The index in each case can again be seen moving generally towards normal, consistent with physician observation of the patients as responding to the new treatment.

Each of FIGS. 21-23 shows the inflammation index for an international group of subjects, suffering from rheumatoid arthritis, each of whom has been characterized as stable (that is, not anticipated to be subjected to a change in therapy) by the subject's treating physician. FIG. 21 shows the index for each of 10 patients in the group being treated with methotrexate, which known to alleviate symptoms without addressing the underlying disease. FIG. 22 shows the index for each of 10 patients in the group being treated with Enbrel (an TNF inhibitor), and FIG. 23 shows the index for each 10 patients being treated with Remicade (another TNF inhibitor). It can be seen that the inflammation index for each of the patients in FIG. 21 is elevated compared to normal, whereas in FIG. 22, the patients being treated with Enbrel as a class have an inflammation index that comes much closer to normal (80% in the normal range). In FIG. 23, it can be seen that, while all but one of the patients being treated with Remicade have an inflammation index at or below normal, two of the patients have an abnormally low inflammation index, suggesting an immunosuppressive, response to this drug. (Indeed, studies have shown that Remicade has been associated with serious infections in some subjects, and here the immunosuppressive effect is quantified.) Also in FIG. 23, one subject has an inflammation index that is significantly above the normal range. This subject in fact was also on a regimen of an anti-inflammation steroid (prednisone) that was being tapered; within approximately one week after the inflammation index was sampled, the subject experienced a significant flare of clinical symptoms.

Remarkably, these examples show a measurement, derived from the assay of blood taken from a subject, pertinent to the subject's arthritic condition. Given that the measurement pertains to the extent of inflammation, it can be expected that other inflammation-based conditions, including, for example, cardiovascular disease, may be monitored in a similar fashion.

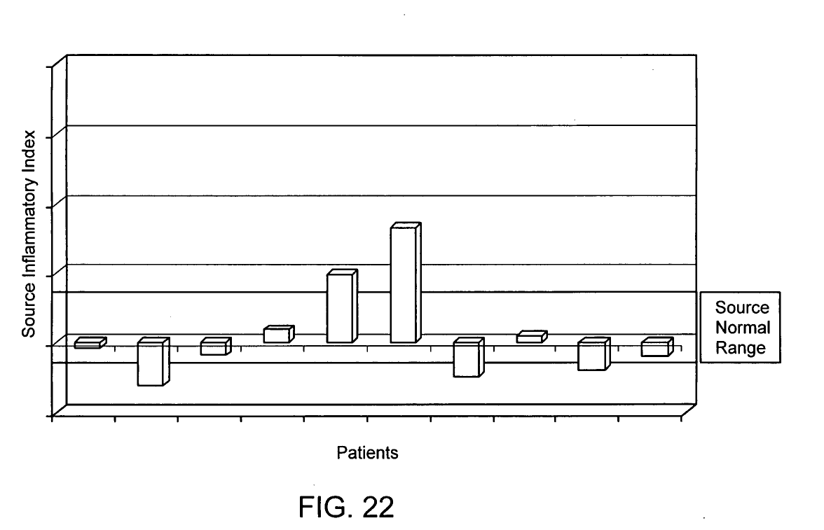

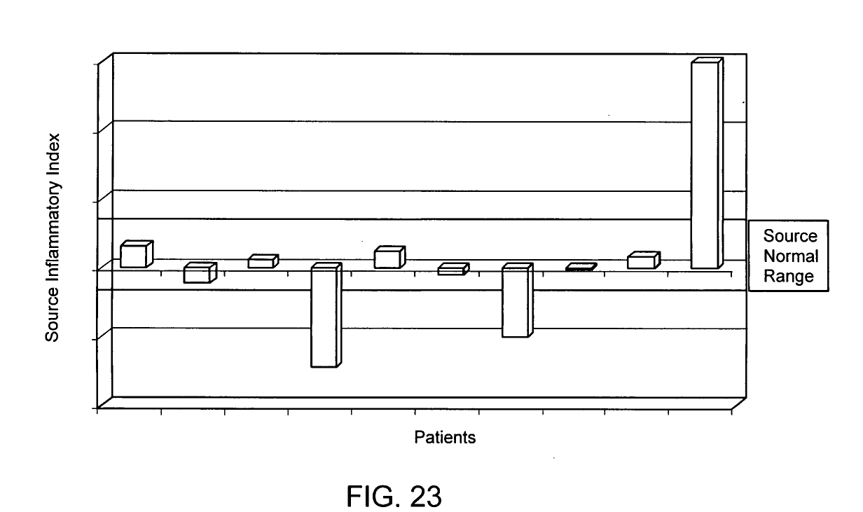

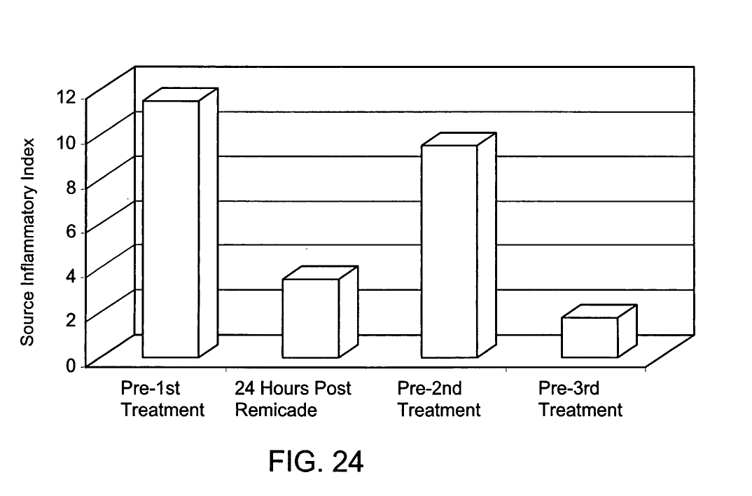

FIG. 24 illustrates use of the inflammation index for assessment of a single subject suffering from inflammatory bowel disease, for whom treatment with Remicade was initiated in three doses. The graphs show the inflammation index just prior to first treatment, and then 24 hours after the first treatment; the index has returned to the normal range. The index was elevated just prior to the second dose, but in the normal range prior to the third dose. Again, the index, besides providing a measure of biological condition, is here used to measure the effectiveness of therapy (Remicade), as well as to provide a target for therapeutic intervention in terms of both dose and schedule.

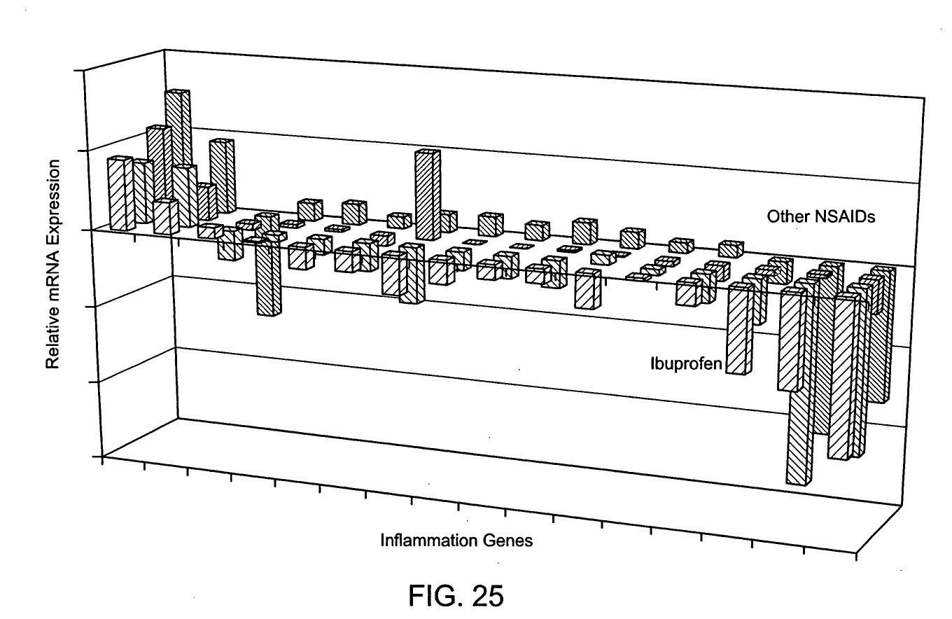

FIG. 25 shows Gene Expression Profiles with respect to 24 loci (of the Inflammation Gene Expression Panel of Table 1) for whole blood treated with Ibuprofen in vitro in relation to other non-steroidal anti-inflammatory drugs (NSADDs). The profile for Ibuprofen is in front. It can be seen that all of the NSAIDs, including Ibuprofen share a substantially similar profile, in that the patterns of gene expression across the loci are similar. Notwithstanding these similarities, each individual drug has its own distinctive signature.

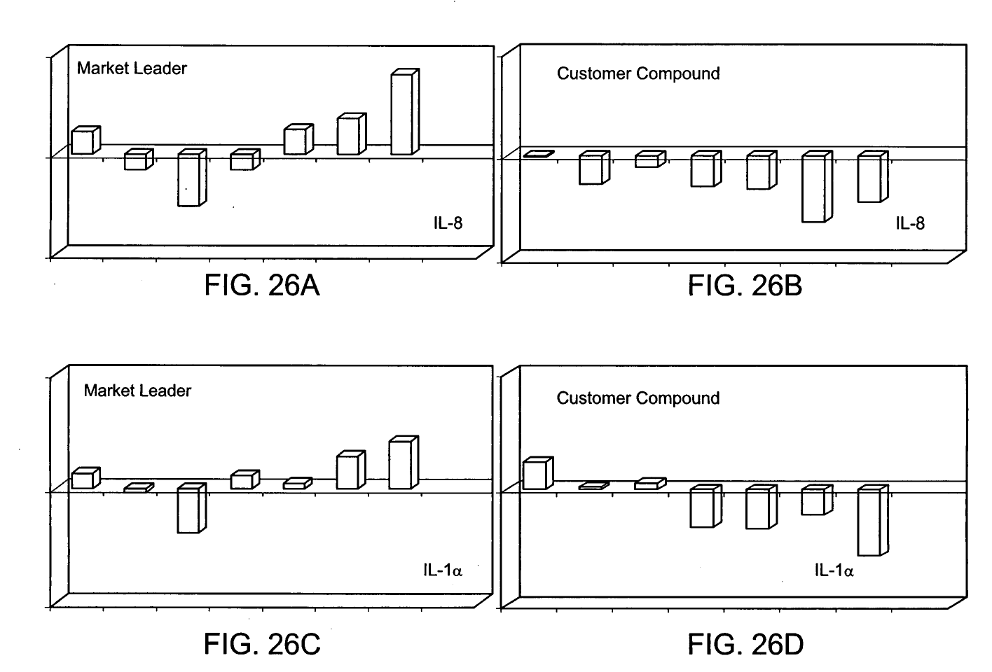

FIG. 26 illustrates how the effects of two competing anti-inflammatory compounds can be compared objectively, quantitatively, precisely, and reproducibly. In this example, expression of each of a panel of two genes (of the Inflammation Gene Expression Panel of Table 1) is measured for varying doses (0.08-250 μg/ml) of each drug in vitro in whole blood. The market leader drug shows a complex relationship between dose and inflammatory gene response. Paradoxically, as the dose is increased, gene expression for both loci initially drops and then increases in the case the case of the market leader. For the other compound, a more consistent response results, so that as the dose is increased, the gene expression for both loci decreases more consistently.

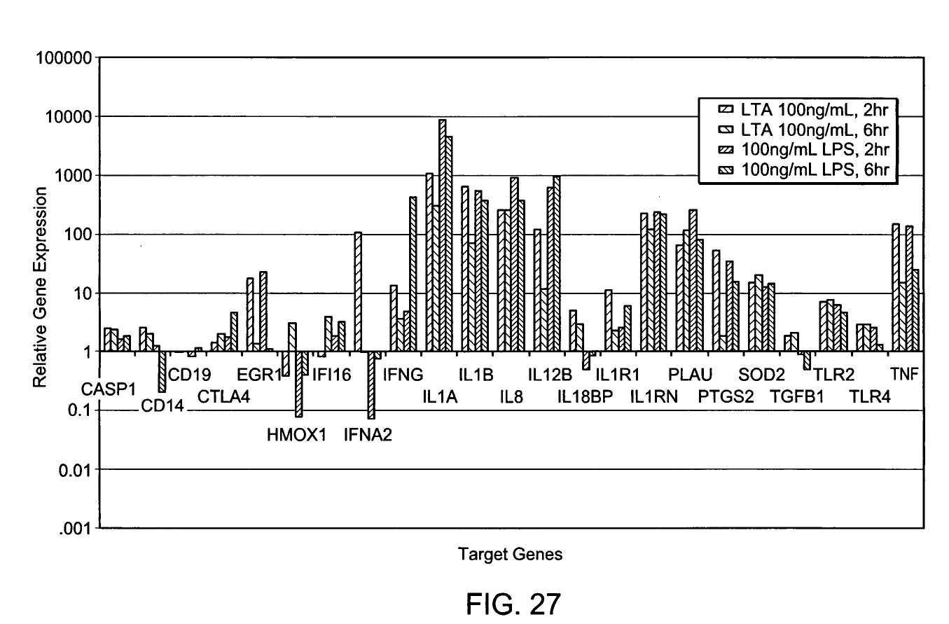

FIGS. 27 through 41 illustrate the use of gene expression panels in early identification and monitoring of infectious disease. These figures plot the response, in expression products of the genes indicated, in whole blood, to the administration of various infectious agents or products associated with infectious agents. In each figure, the gene expression levels are “calibrated”, as that term is defined herein, in relation to baseline expression levels determined with respect to the whole blood prior to administration of the relevant infectious agent. In this respect the figures are similar in nature to various figures of our below-referenced patent application WO 01/25473 (for example, FIG. 15 therein). The concentration change is shown ratiometrically, and the baseline level of 1 for a particular gene locus corresponds to an expression level for such locus that is the same, monitored at the relevant time after addition of the infectious agent or other stimulus, as the expression level before addition of the stimulus. Ratiometric changes in concentration are plotted on a logarithmic scale. Bars below the unity line represent decreases in concentration and bars above the unity line represent increases in concentration, the magnitude of each bar indicating the magnitude of the ratio of the change. We have shown in WO 01/25473 and other experiments that, under appropriate conditions, Gene Expression Profiles derived in vitro by exposing whole blood to a stimulus can be representative of Gene Expression Profiles derived in vivo with exposure to a corresponding stimulus.

FIG. 27 uses a novel bacterial Gene Expression Panel of 24 genes, developed to discriminate various bacterial conditions in a host biological system. Two different stimuli are employed: lipotechoic acid (LTA), a gram positive cell wall constituent, and lipopolysaccharide (LPS), a gram negative cell wall constituent. The final concentration immediately after administration of the stimulus was 100 ng/mL, and the ratiometric changes in expression, in relation to pre-administration levels, were monitored for each stimulus 2 and 6 hours after administration. It can be seen that differential expression can be observed as early as two hours after administration, for example, in the IFNA2 locus, as well as others, permitting discrimination in response between gram positive and gram negative bacteria.

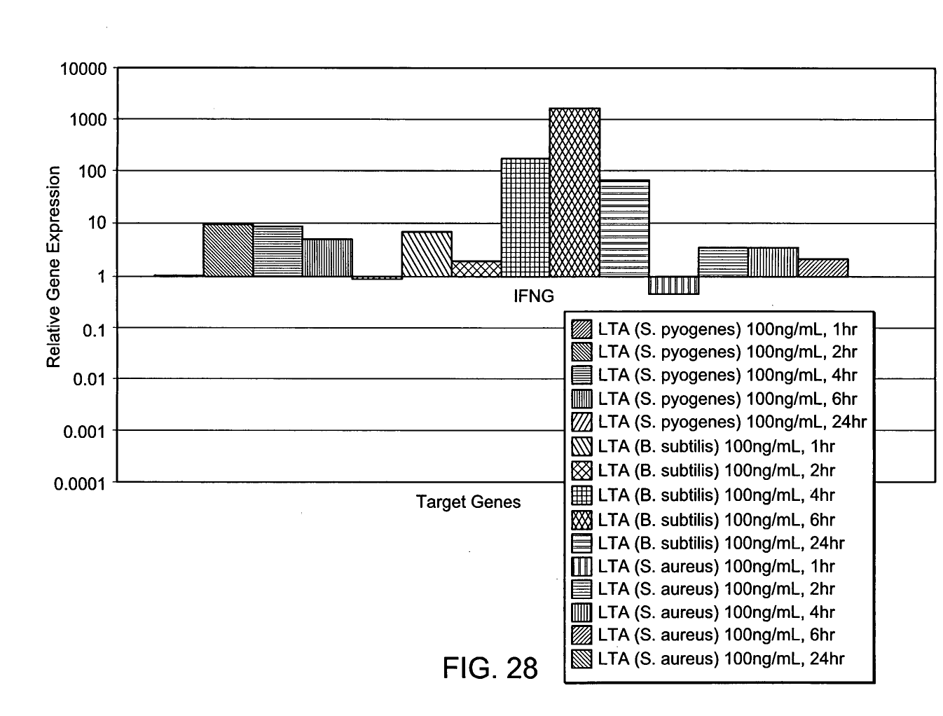

FIG. 28 shows differential expression for a single locus, IFNG, to LTA derived from three distinct sources: S. pyogenes, B. subtilis, and S. aureus. Each stimulus was administered to achieve a concentration of 100 ng/mL, and the response was monitored at 1, 2, 4, 6, and 24 hours after administration. The results suggest that Gene Expression Profiles can be used to distinguish among different infectious agents, here different species of gram positive bacteria.

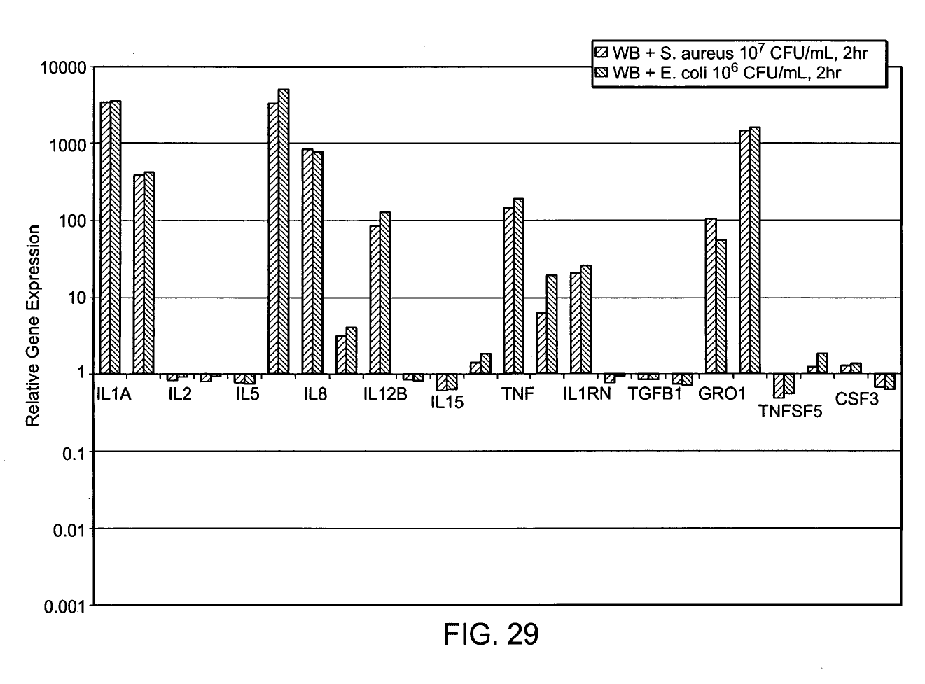

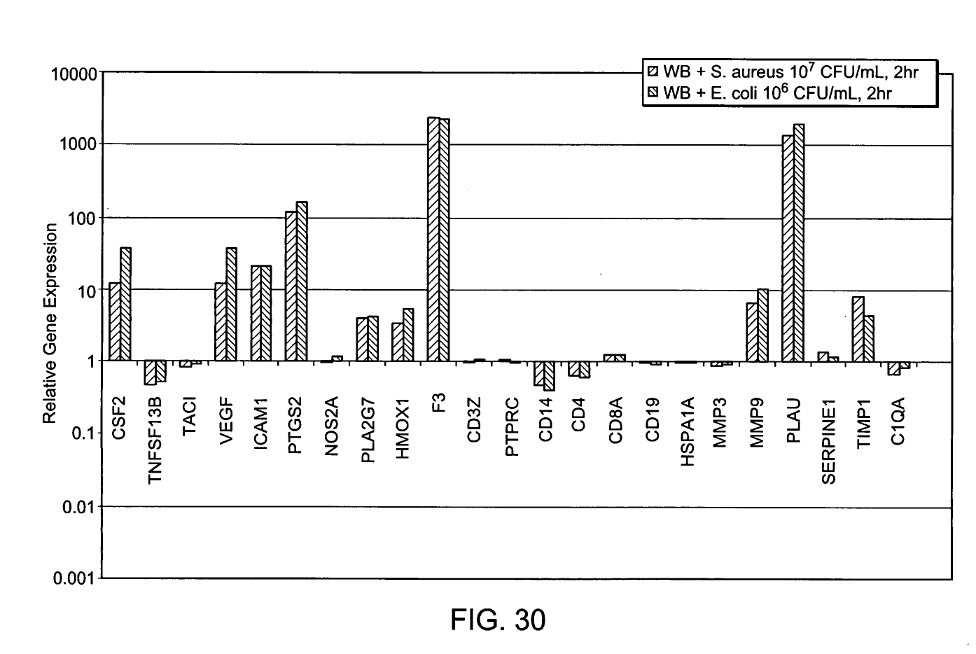

FIGS. 29 and 30 show the response of the Inflammation 48A and 48B loci respectively (discussed above in connection with FIGS. 6 and 7 respectively) in whole blood to administration of a stimulus of S. aureus and of a stimulus of E. coli (in the indicated concentrations, just after administration, of 10⁷ and 10⁶ CFU/mL respectively), monitored 2 hours after administration in relation to the pre-administration baseline. The figures show that many of the loci respond to the presence of the bacterial infection within two hours after infection.

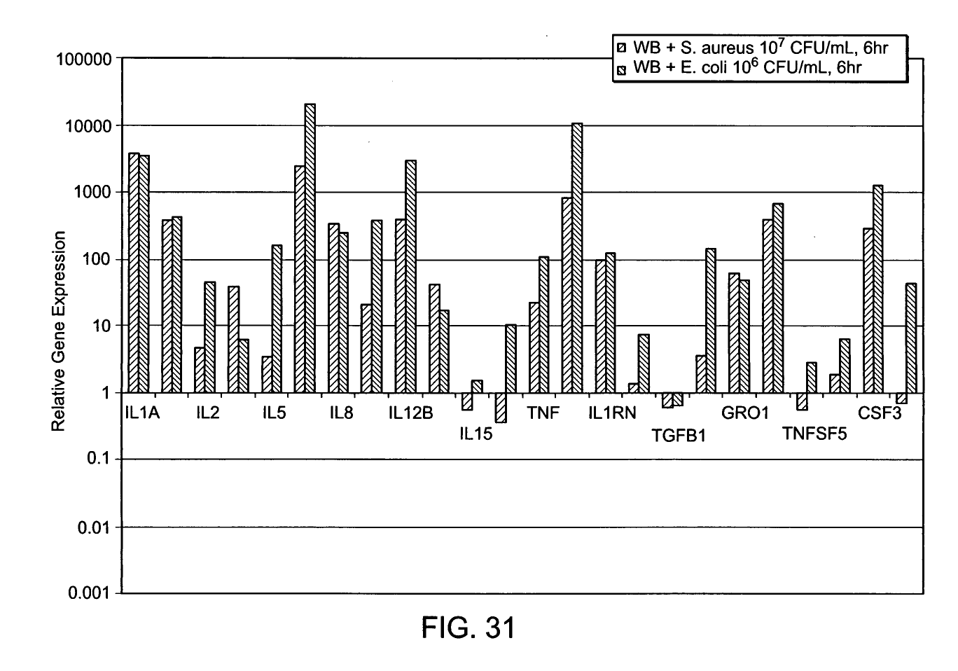

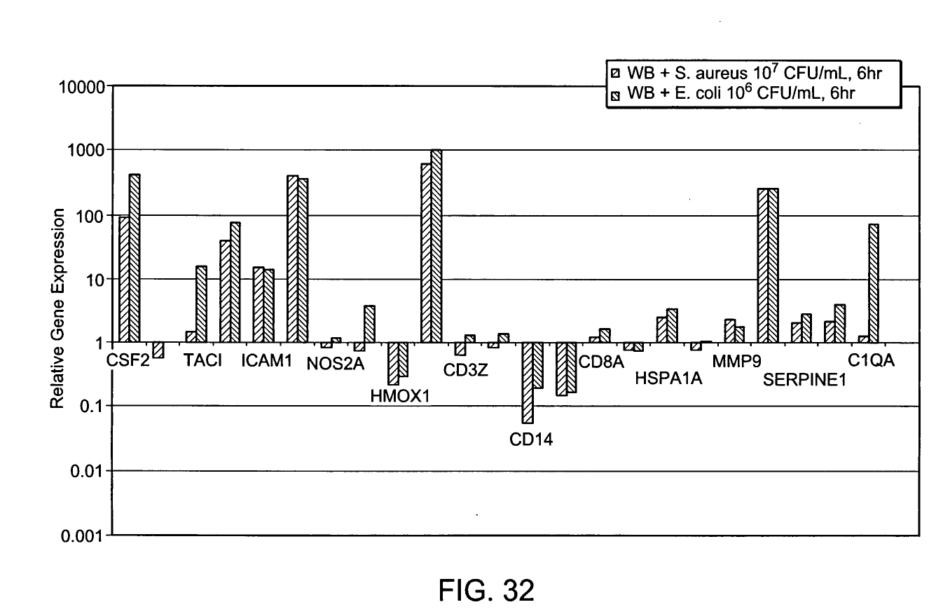

FIGS. 31 and 32 correspond to FIGS. 29 and 30 respectively and are similar to them, with the exception that the monitoring here occurs 6 hours after administration. More of the loci are responsive to the presence of infection. Various loci, such as IL2, show expression levels that discriminate between the two infectious agents.

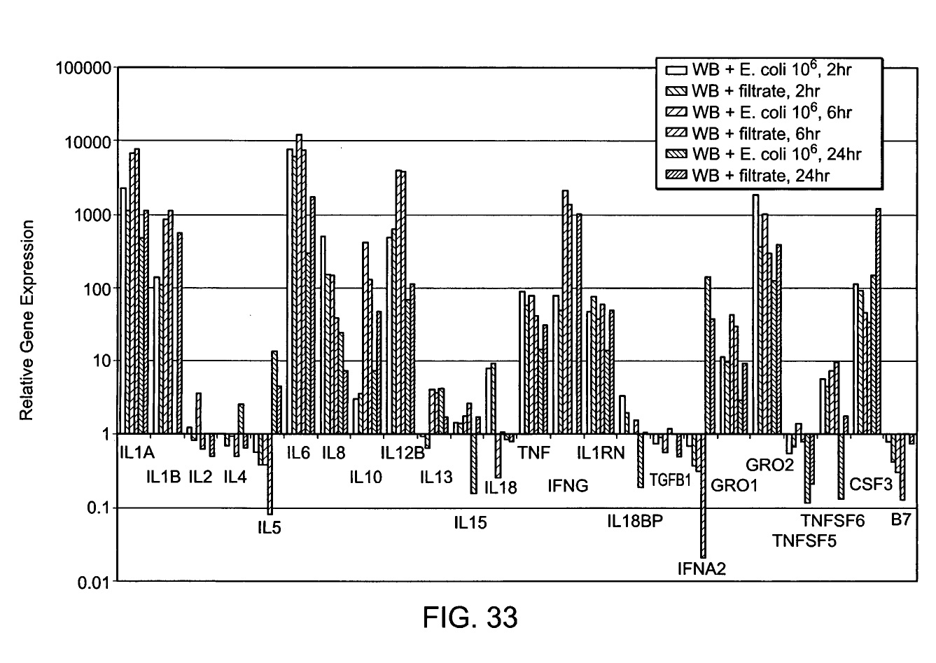

FIG. 33 shows the response of the Inflammation 48A loci to the administration of a stimulus of E. coli (again in the concentration just after administration of 10⁶ CFU/mL) and to the administration of a stimulus of an E. coli filtrate containing E. coli bacteria by products but lacking E. coli bacteria. The responses were monitored at 2, 6, and 24 hours after administration. It can be seen, for example, that the responses over time of loci IL1B, IL18 and CSF3 to E.coli and to E. coli filtrate are different.

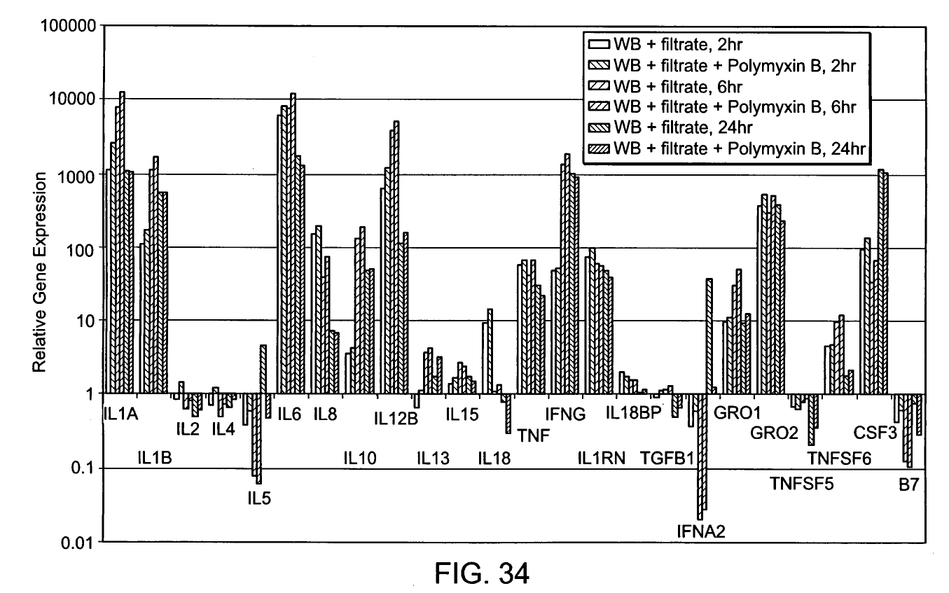

FIG. 34 is similar to FIG. 33, but here the compared responses are to stimuli from E. coli filtrate alone and from E. coli filtrate to which has been added polymyxin B, an antibiotic known to bind to lipopolysaccharide (LPS). An examination of the response of IL1B, for example, shows that presence of polymyxin B did not affect the response of the locus to E. coli filtrate, thereby indicating that LPS does not appear to be a factor in the response of IL1B to E. coli filtrate.

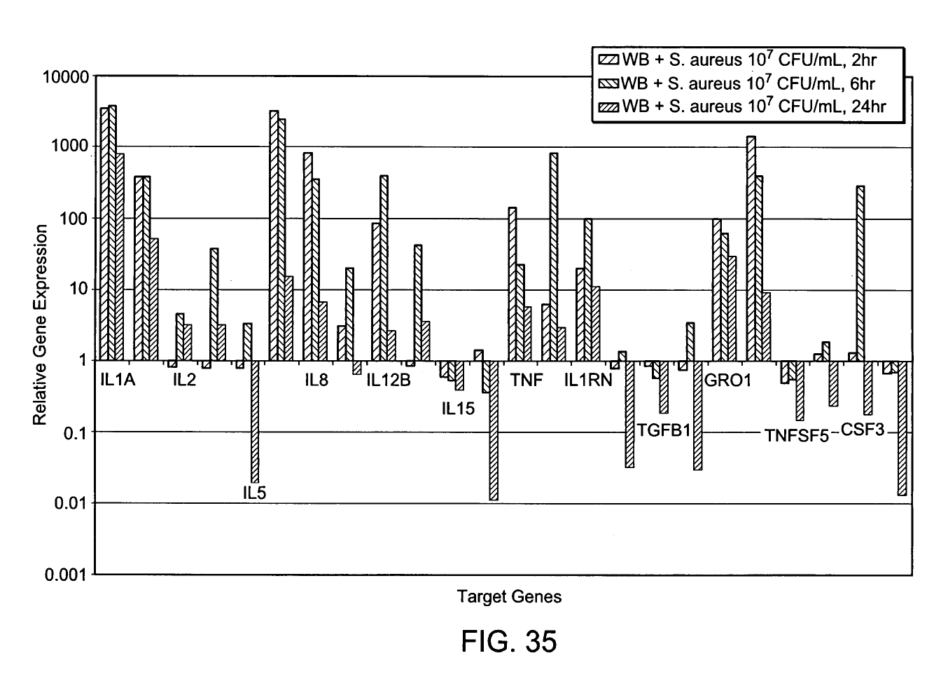

FIG. 35 illustrates the responses of the Inflammation 48A loci over time of whole blood to a stimulus of S. aureus (with a concentration just after administration of 10⁷ CFU/mL) monitored at 2, 6, and 24 hours after administration. It can be seen that response over time can involve both direction and magnitude of change in expression. (See for example, IL5 and IL18.)

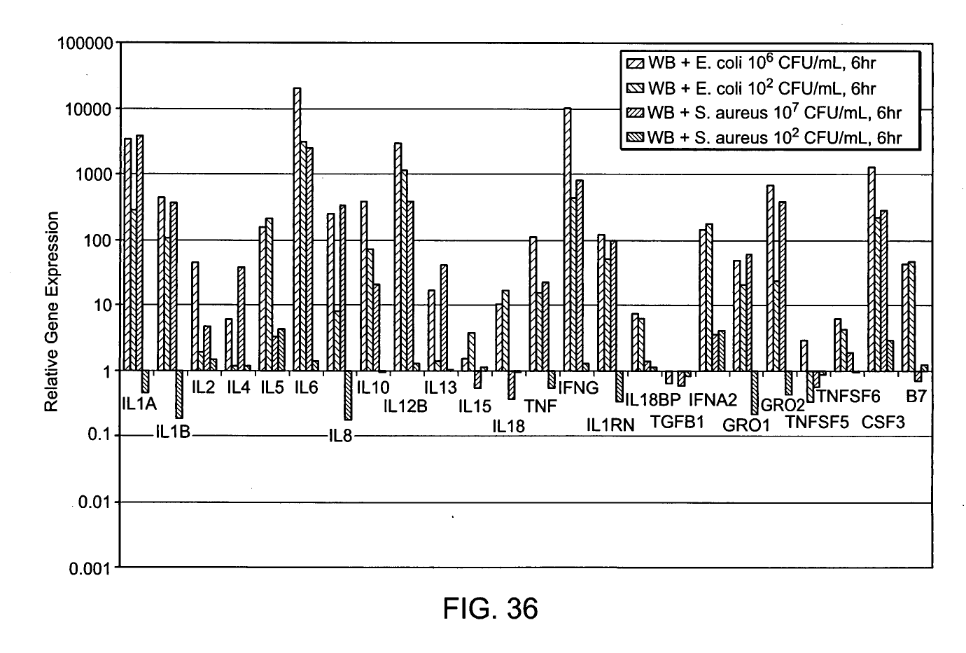

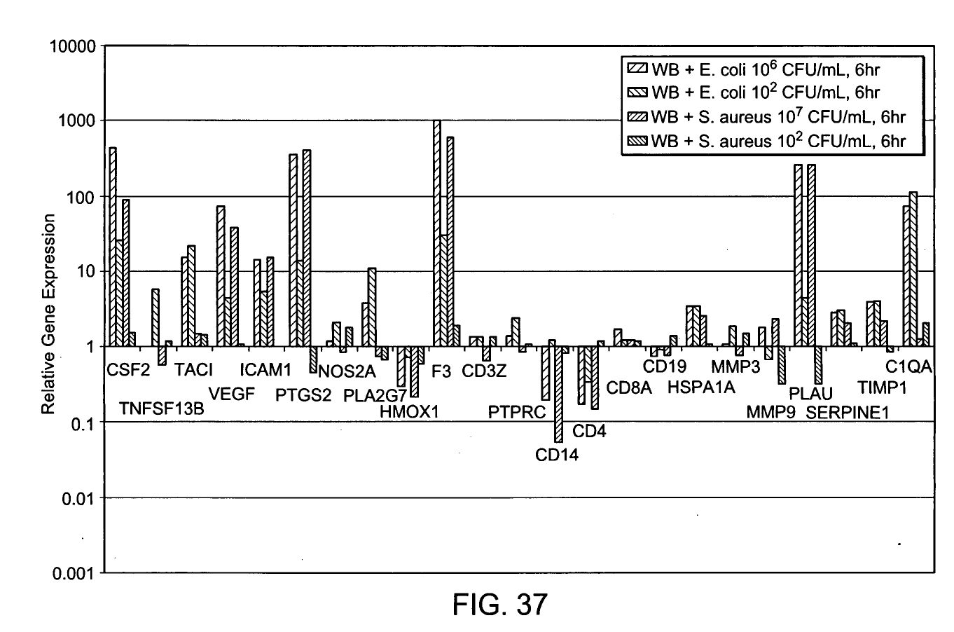

FIGS. 36 and 37 show the responses, of the Inflammation 48A and 48B loci respectively, monitored at 6 hours to stimuli from E. coli (at concentrations of 10⁶ and 10² CFU/mL immediately after administration) and from S. aureus (at concentrations of 10⁷ and 10² CFU/mL immediately after administration). It can be seen, among other things, that in various loci, such as B7 (FIG. 36), TACI, PLA2G7, and C1QA (FIG. 37), E. coli produces a much more pronounced response than S. aureus. The data suggest strongly that Gene Expression Profiles can be used to identify with high sensitivity the presence of gram negative bacteria and to discriminate against gram positive bacteria.

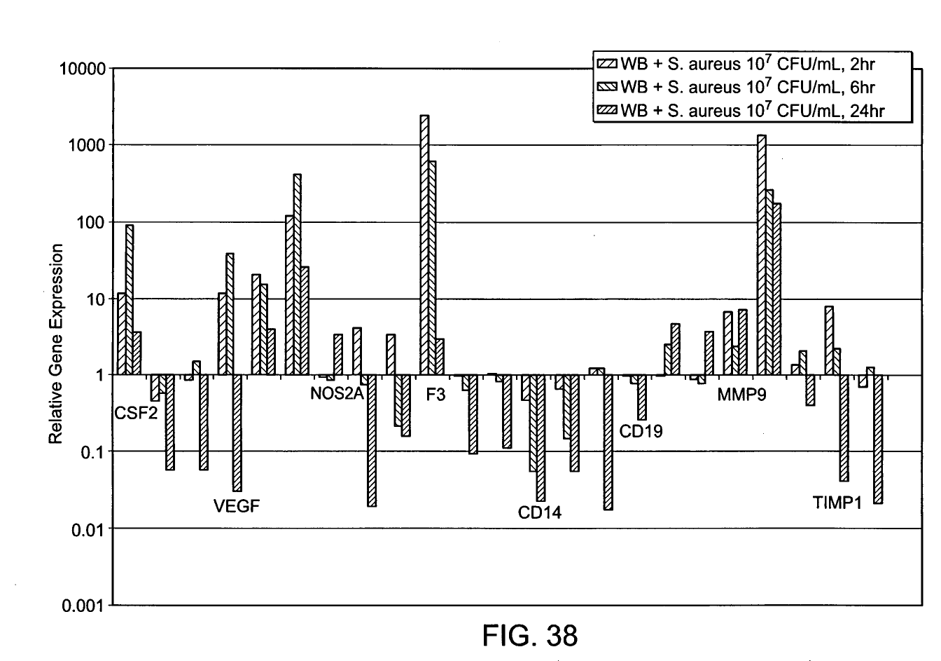

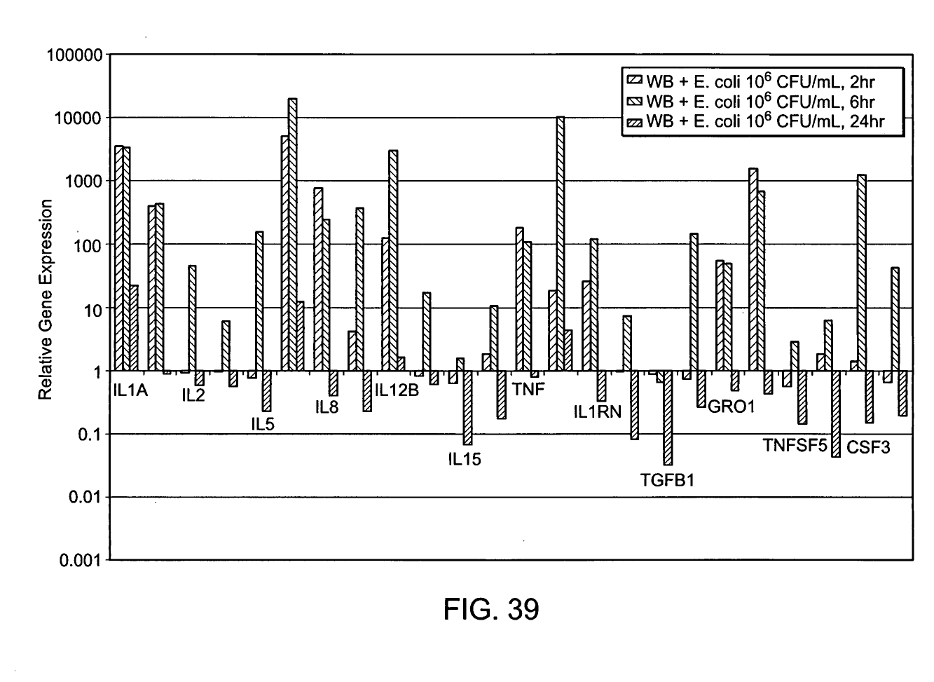

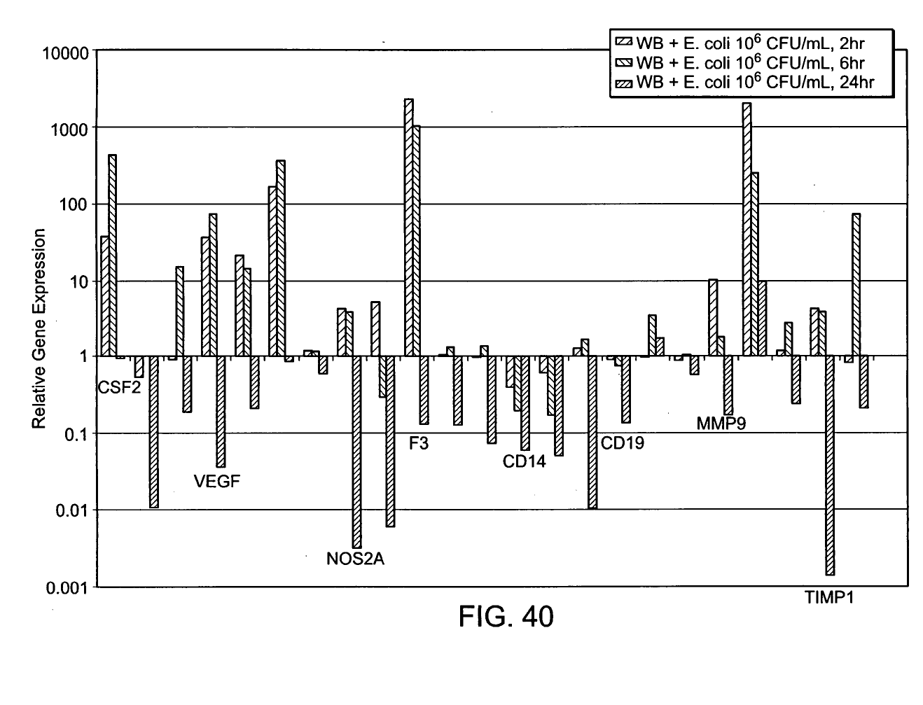

FIGS. 38 and 39 show the responses, of the Inflammation 48B and 48A loci respectively, monitored 2, 6, and 24 hours after administration, to stimuli of high concentrations of S. aureus and E. coli respectively (at respective concentrations of 10⁷ and 10⁶ CFU/mL immediately after administration). The responses over time at many loci involve changes in magnitude and direction. FIG. 40 is similar to FIG. 39, but shows the responses of the Inflammation 48B loci.

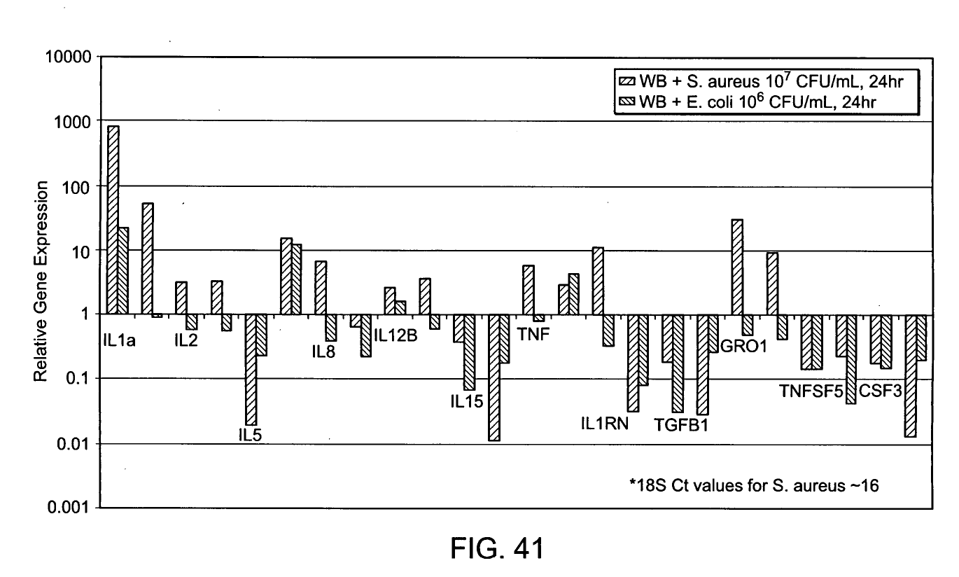

FIG. 41 similarly shows the responses of the Inflammation 48A loci monitored at 24 hours after administration to stimuli high concentrations of S. aureus and E. coli respectively (at respective concentrations of 10⁷ and 10⁶ CFU/mL immediately after administration). As in the case of FIGS. 20 and 21, responses at some loci, such as GRO1 and GRO2, discriminate between type of infection.

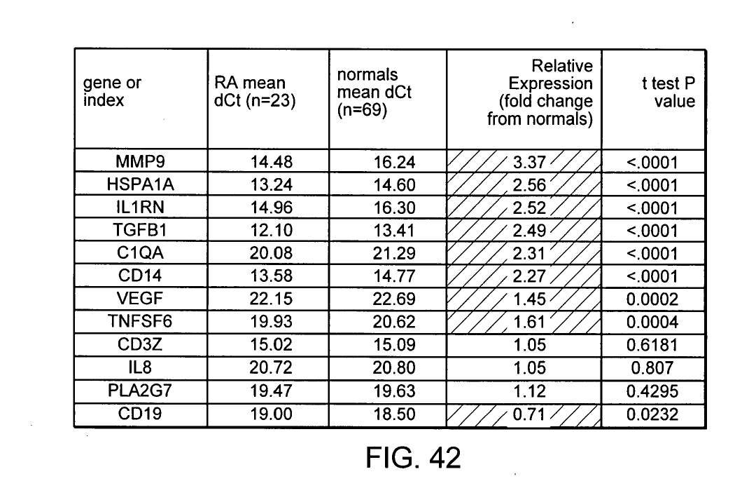

FIG. 42 illustrates application of a statistical T-test to identify potential members of a signature gene expression panel that is capable of distinguishing between normal subjects and subjects suffering from unstable rheumatoid arthritis. The grayed boxes show genes that are individually highly effective (t test P values noted in the box to the right in each case) in distinguishing between the two sets of subjects, and thus indicative of potential members of a signature gene expression panel for rheumatoid arthritis.

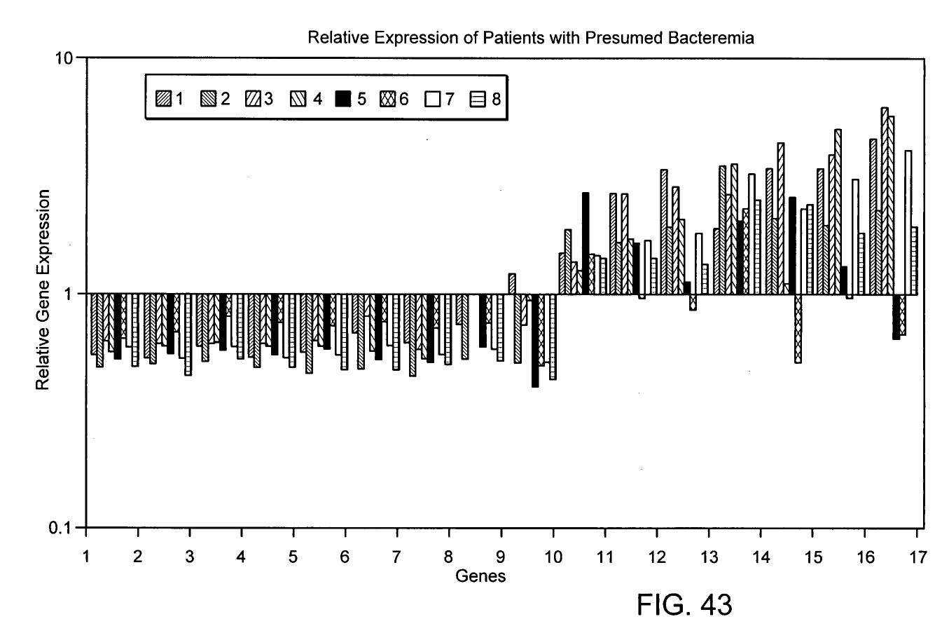

FIG. 43 illustrates, for a panel of 17 genes, the expression levels for 8 patients presumed to have bacteremia. The data are suggestive of the prospect that patients with bacteremia have a characteristic pattern of gene expression.

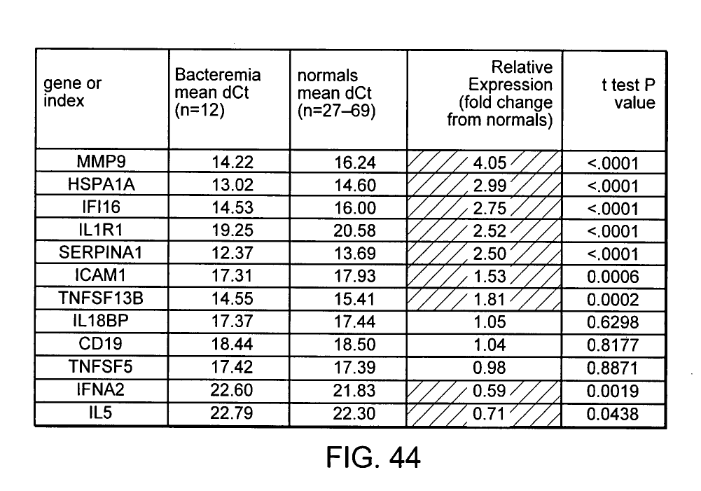

FIG. 44 illustrates application of a statistical T-test to identify potential members of a signature gene expression panel that is capable of distinguishing between normal subjects and subjects suffering from bacteremia. The grayed boxes show genes that are individually highly effective (t test P values noted in the box to the right in each case) in distinguishing between the two sets of subjects, and thus indicative of potential members of a signature gene expression panel for bacteremia.

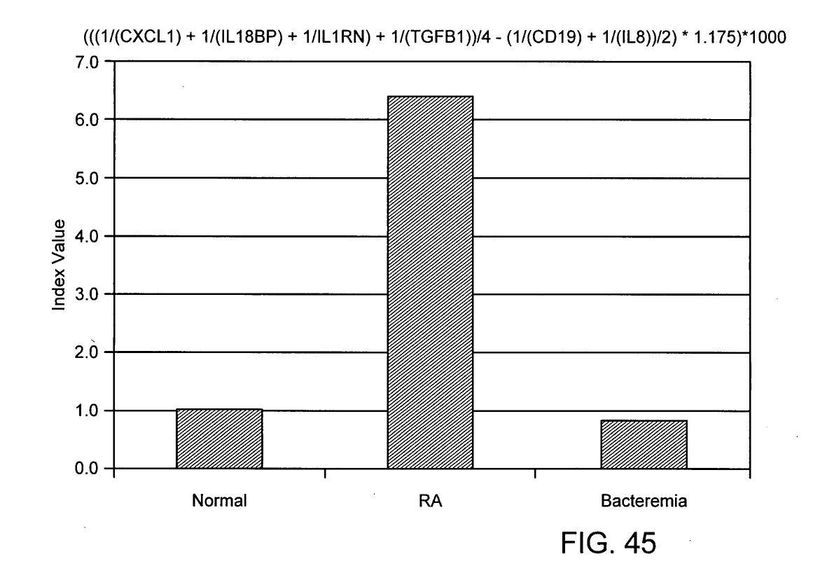

FIG. 45 illustrates application of an algorithm (shown in the figure), providing an index pertinent to rheumatoid arthritis (RA) as applied respectively to normal subjects, RA patients, and bacteremia patients. The index easily distinguishes RA subjects from both normal subjects and bacteremia subjects.

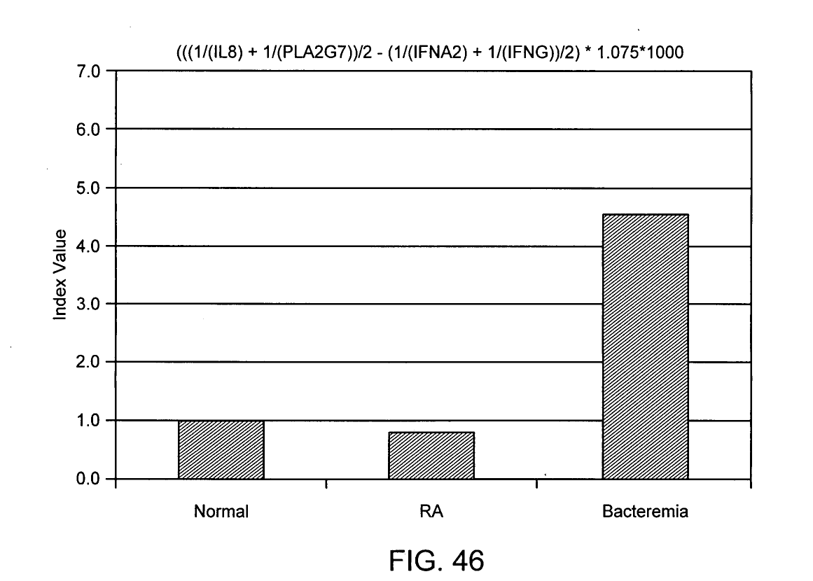

FIG. 46 illustrates application of an algorithm (shown in the figure), providing an index pertinent to bacteremia as applied respectively to normal subjects, rheumatoid arthritis patients, and bacteremia patients. The index easily distinguishes bacteremia subjects from both normal subjects and rheumatoid arthritis subjects.

### Example 7

A female subject with a long, documented history of relapsing, remitting multiple sclerosis sought medical attention from a neurologist for increasing lower trunk muscle weakness (Visit 1, May 22, 2002). Blood was drawn for several assays and the subject was given 5 mg prednisone at that visit. Increasing weakness and spreading of the involvement caused subject to return to the neurologist 6 days later. Blood was drawn and the subject was started on 100 mg prednisone and tapered to 5 mg over one week. The subject reported that her muscle weakness subsided rapidly. The subject was seen for a routine visit (visit 3) more than 2 months later (Jul. 15, 2002). The patient reported no signs of illness at that visit.

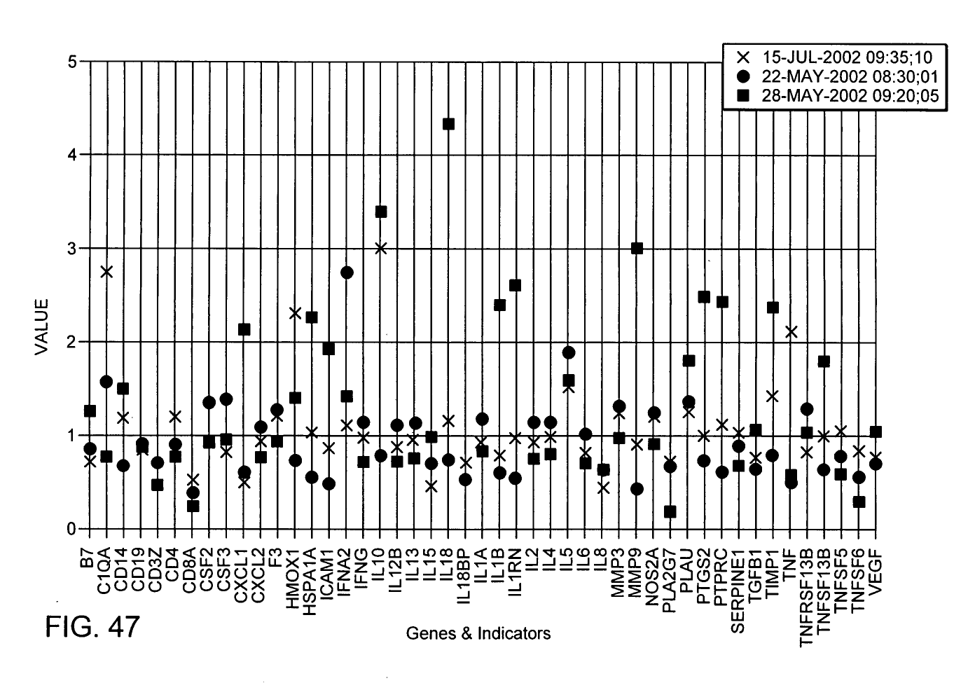

Results of high precision gene expression analysis are shown below in FIG. 47. The “y” axis reports the gene expression level in standard deviation units compared to the Source Precision Medicine Normal Reference Population Value for that gene locus at dates May 22, 2002 (before prednisone treatment), May 28, 2002 (after 5 mg treatment on May 22) and Jul. 15, 2002 (after 100 mg prednisone treatment on May 28, tapering to 5 mg within one week). Expression Results for several genes from the 73 gene locus Multiple Sclerosis Precision Profile (selected from genes in Table 1) are shown along the “x” axis. Some gene loci, for example IL18; IL1B; MMP9; PTGS2, reflect the severity of the signs while other loci, for example IL10, show effects induced by the steroid treatment. Other loci reflect the non-relapsing TIMP1; TNF; HMOX1.

### Example 8

Samples of whole blood from patients with relapsing remitting multiple sclerosis (RRMS) are collected while their disease is clinically inactive. Additional samples are collected during a clinical exacerbation of the MS (or attack). Levels of gene expression of mediators of inflammatory processes are examined before, during, and after the episode, whether or not anti-inflammatory treatment is employed. The post-attack samples are then compared to samples obtained at baseline and those obtained during the exacerbation, prior to initiation of any anti-inflammatory medication. The results of this study are then compared to a database of normal subjects to identify and select diagnostic and prognostic markers of MS activity to be used in Gene Expression Panels for characterizing and evaluating MS according to described embodiments. Selected markers are then tested in additional trials in patients known to have MS, and those suspected of having MS. By using genes selected to be especially probative in characterizing MS and inflammation related to MS, such conditions may be identified in patients using the herein-described gene expression profile techniques and methods of characterizing multiple sclerosis or inflammatory conditions related to multiple sclerosis in a subject based on a sample from the subject. In such a way it is possible to evaluate, diagnose and characterize MS and inflammatory conditions related to MS in a subject, or population of subjects.

In this system, RRMS subjects experiencing a clinical exacerbation will show altered inflammatory-immune response gene expression compared to RRMS patients during remission and healthy subjects. Additionally, gene expression changes will be evident in patients who have exacerbations coincident with initiation and completion of treatment.

This system thus provides a gene expression assay system for monitoring MS patients that is predictive of disease progression and treatment responsiveness. In using this system, gene expression profile data sets are determined and prepared from inflammation and immune-response related genes (mRNA and protein) in whole blood samples taken from RRMS patients before, during and after clinical exacerbation. Samples taken during an exacerbation are collected prior to treatment for the attack. Gene expression results are then correlated with relevant clinical indices as described.

In addition, the observed data in the gene expression profile data sets is compared to reference profile data sets determined from samples from undiagnosed healthy subjects (normals), gene expression profiles for other chronic immune-related genes, and to profile data sets determined for the individual patients during and after the attack. If desired, a subset of the selected identified genes is coupled with appropriate predictive biomedical algorithms for use in predicting and monitoring RRMS disease activity.

In particular embodiments, a study is conducted with approximately 15-20 patients, or 50 to 100 patients. Patients are required to have an existing diagnosis of RRMS and be clinically stable for at least thirty days prior to enrollment. They may be using disease-modifying medication (Interferon or Glatirimer Acetate). All patients are sampled at baseline, defined as a time when the subject is not currently experiencing an attack (see inclusion criteria). Those who experience significant neurological symptoms, suggestive of a clinical exacerbation, are sampled prior to any treatment for the attack. If the patient is found to have a clinical exacerbation, then a repeat sample is obtained four weeks later, regardless of whether the patient receives steroids or other treatment for the exacerbation.

A clinical exacerbation is defined as the appearance of a new symptom or worsening/reoccurrence of an old symptom, attributed to RRMS, lasting at least 24 hours in the absence of fever, and preceded by stability or improvement for at least 30 days.

Each subject is asked to provide a complete medical history including any existing laboratory test results (i.e. MRI, EDSS scores, blood chemistry, hematology, etc) relevant to the patient's MS contained within the patient's medical records. Additional test results (ordered while the subject is enrolled in the study) relating to the treatment of the patient's MS are collected and correlated with gene expression analysis.

Subjects in the study meet all of the following criteria:

1. Male or Female subjects at least 18 years old with clinically documented active Relapsing-Remitting MS (RRMS) characterized by clearly defined acute attacks followed by full or partial recovery to the pre-existing level of disability, and by a lack of disease progression in the periods between attacks.

2. Subjects are clinically stable for a minimum of 30 days or for a time period determined at the clinician's discretion.

3. Patients are stable (at least three-months) on Interferon therapy or Glatiramer Acetate or are therapy naïve or without the above mentioned therapy for 4 weeks.

4. Subjects must be willing to give written informed consent and to comply with the requirements of the study protocol.

Subjects are excluded from the study if they meet any of the following criteria:

1. Primary progressive multiple sclerosis (PPMS).

2. Immunosuppressive therapy (such as azathioprine and MTX) within three months of study participation. Subjects having prior treatment with cyclophosphamide, total lymphoid irradiation, mitoxantrone, cladribine, or bone marrow transplantation, regardless of duration, are also excluded.

3. Corticosteroid therapy within four weeks of participation of the study.

4. Use of any investigational drug with the intent to treat MS or the symptoms of MS within six months of participation in this trial (agents for the symptomatic treatment of MS, e.g., 4-aminopyridine <4-AP>, may be allowed following discussion with Clinician).

5. Infection or risk factors for severe infections, including: excessive immunosuppression including human immunodeficiency virus (HIV) infection; severe, recurrent, or persistent infections (such as Hepatitis B or C, recurrent urinary tract infection or pneumonia); evidence of current inactive or active tuberculosis (TB) infection including recent exposure to M. tuberculosis (converters to a positive purified protein derivative); subjects with a positive PPD or a chest X-ray suggestive of prior TB infection; active Lyme disease; active syphilis; any significant infection requiring hospitalization or IV antibiotics in the month prior to study participation; infection requiring treatment with antibiotics in the two weeks prior to study participation.

6. Any of the following risk factors for development of malignancy: history of lymphoma or leukemia; treatment of cutaneous squamous-cell or basal cell carcinoma within 2 years of enrollment into the study; other malignancy within 5 years; disease associated with an increased risk of malignancy.

7. Other diseases (in addition to MS) that produce neurologic manifestations, such as amyotrophic lateral sclerosis, Gullain-Barre syndrome, muscular dystrophy, etc.)

8. Pregnant or lactating females.

### Example 9

In other embodiments, studies are designed to identify possible markers of disease activity in multiple sclerosis (MS) to aid in selecting genes for particular Gene Expression Panels. Similar to the previously-described example, the results of this study are compared to a database of gene expression profile data sets determined and obtained from samples from healthy subjects, and the results are used to identify possible markers of MS activity to be used in Gene Expression Panels for characterizing and evaluating MS according to described embodiments. Selected markers are then tested in additional trials to assess their predictive value.

Approximately 30 patients are used this study, although other studies may use 50 or 100 subjects. Initially, a smaller number of patients are evaluated, and gene expression profile data sets are determined for these patients and the expression profiles of selected inflammatory markers are assessed. Additional subjects are added to the study after preliminary evidence for particular disease activity markers is obtained so that a larger or more particular panel of genes is selected for determining profile data sets for the full number of subjects in the study.

Patients who are not receiving disease-modifying therapy such as interferon are of particular interest but inclusion of patients receiving such therapy is also useful. Patients are asked to give blood at two timepoints—first at enrollment and then again at 3-12 months after enrollment. Clinical data relating to present and history of disease activity, concomitant medications, lab and MRI results, as well as general health assessment questionnaires may be also be collected.

In this embodiment, patients meeting the following specific criteria are desirable for the study:

1. Patients having MS that meets the criteria of McDonald et al.

2. Patients with clinically active disease as shown by ≧1 exacerbation in previous 12 months.

3. Patients not in acute relapse

4. Patients willing to provide up to 10 ml of blood at up to 3 time points In addition, patients with known hepatitis or HIV infection are not eligible. The enrollment samples from suitable subjects were collected prior to the patient receiving any disease modifying therapy. The later samples are collected 3-12 months after the patients start therapy. Preliminary data suggests that gene expression may be used to track drug response and that only a plurality or several genetic markers is required to identify MS in a population of samples.

### Example 10

Yet another embodiment provides a study for identify biomarkers for use in a specific Gene Expression Panel for MS, wherein the genes/biomarkers are selected to evaluate dosing and safety of a new compound developed for treating MS, and to track drug response. The embodiment provides a multi-center, randomized, double blind, placebo-controlled trial to evaluate a new drug therapy in patients with multiple sclerosis.

As in above examples, 20 to 30 subjects are enrolled in this study, or alternatively 50 or 100 subjects or more. Only patients who exhibit stable MS for three months prior to the study are selected for the trial. Stable disease is defined as the absence of progression and relapse. Subjects enrolled in this study have been removed from disease modifying therapy for at least 1 month. A subject's clinical status is monitored throughout the study by MRI and hematology and blood chemistries.

Throughout the study patients receive all medications necessary for management of their MS, including high-dose corticosteroids for management of relapses and introduction of standard treatments for MS. Initiation of such treatments will confound assessment of the trial's endpoints. Consequently, patients who require such treatment will be removed from the new drug therapy phase of the trial but will continue to be followed for safety, immune response, and gene expression.

Blood samples for gene expression analysis are collected at screening/baseline (prior to initiation of drug), several times during the treatment phase and several times during follow-up (post-treatment phase). Gene expression results are compared within subjects, between subjects, and to Source Precision Medicine profile data sets determined to be what are termed “Normals”—i.e., a baseline profile dataset determined for a population of healthy (undiagnosed) individuals who do not have MS or other inflammatory conditions, disease, infections. The results will also be evaluated to compare and contrast gene expression between different timepoints. This study is used to track individual and population response to the drug, and to correlate clinical symptoms (i.e. disease progression, disease remittance, adverse events) with gene expression.

Baseline samples from a subset of patients have been analyzed. The preliminary data from the baseline samples suggest that that only a plurality of or optionally several specific genetic markers is required to identify MS across a population of samples. The study may also be used to track drug response and clinical endpoints.

### Example 11

Still another embodiment provides a study for testing a new experimental treatment for MS. The study may enroll up to 200 MS subjects or more in a Phase 2, multi-center, randomized, double-blind, parallel group, placebo-controlled, dose finding, safety, tolerability, and efficacy study. Samples for gene expression are collected at baseline and at several timepoints during the study. Samples are compared between subjects, within individual subjects, and to Source Precision Medicine profile data sets determined to be what are termed “Normals”—i.e., a baseline profile dataset determined for a population of healthy (undiagnosed) individuals who do not have MS or other inflammatory conditions, disease, infections. The gene expression profile data sets are then assessed for their ability to track individual response to therapy, for identifying a subset of genes that exhibit altered gene expression in MS and/or are affected by the drug treatment. Clinical data collected during the study include: MRIs, disease progression tests (EDSS, MSFC, ambulation tests, auditory testing, dexterity testing), medical history, concomitant medications, adverse events, physical exam, hematology and chemistry labs, urinalysis, and immunologic testing.

Subjects enrolled in the study are asked to discontinue any MS disease modifying therapies they may be using for their disease for at least 3 months prior to dosing with the study drug or drugs.

These data support our conclusion that Gene Expression Profiles with sufficient precision and calibration as described herein (1) can determine subsets of individuals with a known biological condition, particularly individuals with multiple sclerosis or individuals with inflammatory conditions related to multiple sclerosis; (2) may be used to monitor the response of patients to therapy; (3) may be used to assess the efficacy and safety of therapy; and (4) may used to guide the medical management of a patient by adjusting therapy to bring one or more relevant Gene Expression Profiles closer to a target set of values, which may be normative values or other desired or achievable values. We have shown that Gene Expression Profiles may provide meaningful information even when derived from ex vivo treatment of blood or other tissue. We have also shown that Gene Expression Profiles derived from peripheral whole blood are informative of a wide range of conditions neither directly nor typically associated with blood.

Gene Expression Profiles can be used for characterization and monitoring of treatment efficacy of individuals with multiple sclerosis, or individuals with inflammatory conditions related to multiple sclerosis.

Furthermore, in embodiments of the present invention, Gene Expression Profiles can also be used for characterization and early identification (including pre-symptomatic states) of infectious disease, such as sepsis. This characterization includes discriminating between infected and uninfected individuals, bacterial and viral infections, specific subtypes of pathogenic agents, stages of the natural history of infection (e.g., early or late), and prognosis. Use of the algorithmic and statistical approaches discussed above to achieve such identification and to discriminate in such fashion is within the scope of various embodiments herein.

TABLE 1
Multiple Sclerosis or Inflammatory Conditions Related to Multiple Sclerosis
Gene Expression Panel

| Symbol | Name | Classification | Description |
|---|---|---|---|
| APAF1 | Apoptotic Protease | Protease | Cytochrome c binds to APAF1, triggering |
| | Activating Factor 1 | activating | activation of CASP3, leading to apoptosis. |
| | | peptide | May also facilitate procaspase 9 auto activation. |
| ARG2 | Arginase II | Enzyme/redox | Catalyzes the hydrolysis of arginine to ornithine |
| | | | and urea; may play a role in down regulation of |
| | | | nitric oxide synthesis |
| BCL2 | B-cell CLL/ | Apoptosis | Blocks apoptosis by interfering with the |
| | lymphoma 2 | Inhibitor - cell | activation of caspases |
| | | cycle control - | |
| | | oncogenesis | |
| BPI | Bactericidal/permeability- | Membrane- | LPS binding protein; cytotoxic for many gram |
| | increasing | bound protease | negative organisms; found in myeloid cells |
| | protein | | |
| C1QA | Complement | Proteinase/ | Serum complement system; forms C1 complex |
| | component 1, q | proteinase | with the proenzymes c1r and c1s |
| | subcomponent, alpha | inhibitor | |
| | polypeptide | | |
| CALCA | Calcitonin/calcitonin- | cell-signaling | AKA CALC1; Promotes rapid incorporation of |
| | related poplypeptide, | and activation | calcium into bone |
| | alpha | | |
| CASP1 | Caspase 1 | Proteinase | Activates IL1B; stimulates apoptosis |
| CASP3 | Caspase 3 | Proteinase/ | Involved in activation cascade of caspases |
| | | Proteinase | responsible for apoptosis - cleaves CASP6, |
| | | Inhibitor | CASP7, CASP9 |
| CASP9 | Caspase 9 | Proteinase | Binds with APAF1 to become activated; |
| | | | cleaves and activates CASP3 |
| CCL1 | Chemokine (C—C | Cytokines- | Secreted by activated T cells; chemotactic for |
| | Motif) ligand 1 | chemokines- | monocytes, but not neutrophils; binds to CCR8 |
| | | growth factors | |
| CCL2 | Chemokine (C—C | Cytokines- | CCR2 chemokine; Recruits monocytes to areas |
| | Motif) ligand 2 | chemokines- | of injury and infection; Upregulated in liver |
| | | growth factors | inflammation; Stimulates IL-4 production; |
| | | | Implicated in diseases involving monocyte, |
| | | | basophil infiltration of tissue (e.g. psoriasis, |
| | | | rheumatoid arthritis, atherosclerosis) |
| CCL3 | Chemokine (C—C | Cytokines- | AKA: MIP1-alpha; monkine that binds to |
| | motif) ligand 3 | chemokines- | CCR1, CCR4 and CCR5; major HIV- |
| | | growth factors | suppressive factor produced by CD8 cells. |
| CCL4 | Chemokine (C—C | Cytokines- | Inflammatory and chemotactic monokine; binds |
| | Motif) ligand 4 | chemokines- | to CCR5 and CCR8 |
| | | growth factors | |
| CCL5 | Chemokine (C—C | Cytokines- | Binds to CCR1, CCR3, and CCR5 and is a |
| | Motif) ligand 5 | chemokines- | chemoattractant for blood monocytes, memory |
| | | growth factors | T-helper cells and eosinophils; A major HIV- |
| | | | suppressive factor produced by CD8-positive T- |
| | | | cells |
| CCR1 | chemokine (C—C | chemokine | A member of the beta chemokine receptor |
| | motif) receptor 1 | receptor | family (seven transmembrane protein). Binds |
| | | | SCYA3/MIP-1a, SCYA5/RANTES, MCP-3, |
| | | | HCC-1, 2, and 4, and MPIF-1. Plays role in |
| | | | dendritic cell migration to inflammation sites |
| | | | and recruitment of monocytes. |
| CCR3 | Chemokine (C—C | Chemokine | C—C type chemokine receptor (Eotaxin |
| | motif) receptor 3 | receptor | receptor) binds to Eotaxin, Eotaxin-3, MCP-3, |
| | | | MCP-4, SCYA5/RANTES and mip-1 delta |
| | | | thereby mediating intracellular calcium flux. |
| | | | Alternative co-receptor with CD4 for HIV-1 |
| | | | infection. Involved in recruitment of |
| | | | eosinophils. Primarily a Th2 cell chemokine |
| | | | receptor. |
| CCR5 | chemokine (C—C | chemokine | Binds to CCL3/MIP-1a and CCL5/RANTES. |
| | motif) receptor 5 | receptor | An important co-receptor for macrophage- |
| | | | tropic virus, including HIV, to enter cells. |
| CD14 | CD14 antigen | Cell Marker | LPS receptor used as marker for monocytes |
| CD19 | CD19 antigen | Cell Marker | AKA Leu 12; B cell growth factor |
| CD3Z | CD3 antigen, zeta | Cell Marker | T-cell surface glycoprotein |
| | polypeptide | | |
| CD4 | CD4 antigen (p55) | Cell Marker | Helper T-cell marker |
| CD86 | CD 86 Antigen (cD | Cell signaling | AKA B7-2; membrane protein found in B |
| | 28 antigen ligand) | and activation | lymphocytes and monocytes; co-stimulatory |
| | | | signal necessary for T lymphocyte proliferation |
| | | | through IL2 production. |
| CD8A | CD8 antigen, alpha | Cell Marker | Suppressor T cell marker |
| | polypeptide | | |
| CKS2 | CDC28 protein | Cell signaling | Essential for function of cyclin-dependent |
| | kinase regulatory | and activation | kinases |
| | subunit 2 | | |
| CRP | C-reactive protein | acute phase | the function of CRP relates to its ability to |
| | | protein | recognize specifically foreign pathogens |
| | | | and damaged cells of the host and to initiate |
| | | | their elimination by interacting with |
| | | | humoral and cellular effector systems in the |
| | | | blood |
| CSF2 | Granulocyte- | Cytokines- | AKA GM-CSF; Hematopoietic growth factor; |
| | monocyte colony | chemokines- | stimulates growth and differentiation of |
| | stimulating factor | growth factors | hematopoietic precursor cells from various |
| | | | lineages, including granulocytes, macrophages, |
| | | | eosinophils, and erythrocytes |
| CSF3 | Colony stimulating | Cytokines- | AKA GCSF controls production ifferentiation |
| | factor 3 (granulocyte) | chemokines- | and function of granulocytes. |
| | | growth factors | |
| CXCL3 | Chemokine | Cytokines- | Chemotactic pro-inflammatory activation- |
| | (C—X—C-motif) | chemokines- | inducible cytokine, acting primarily upon |
| | ligand 3 | growth factors | hemopoietic cells in immunoregulatory |
| | | | processes, may also play a role in inflammation |
| | | | and exert its effects on endothelial cells in an |
| | | | autocrine fashion. |
| CXCL10 | Chemokine (C—X—C | Cytokines- | AKA: Gamma IP10; interferon inducible |
| | motif) ligand 10 | chemokines- | cytokine IP10; SCYB10; Ligand for CXCR3; |
| | | growth factors | binding causes stimulation of monocytes, NK |
| | | | cells; induces T cell migration |
| CXCR3 | chemokine (C—X—C | cytokines- | Binds to SCYB10/IP-10, SCYB9/MIG, |
| | motif) receptor 3 | chemokines- | SCYB11/1-TAC. Binding of chemokines to |
| | | growth factors | CXCR3 results in integrin activation, |
| | | | cytoskeletal changes and chemotactic |
| | | | migration. |
| DPP4 | Dipeptidyl-peptidase 4 | Membrane | Removes dipeptides from unmodified, n- |
| | | protein; | terminus prolines; has role in T cell activation |
| | | exopeptidase | |
| DTR | Diphtheria toxin | cell signaling, | Thought to be involved in macrophage- |
| | receptor (heparin- | mitogen | mediated cellular proliferation. DTR is a potent |
| | binding epidermal | | mitogen and chemotactic factor for fibroblasts |
| | growth factor-like | | and smooth muscle cells, but not endothelial |
| | growth factor) | | cells. |
| ELA2 | Elastase 2, neutrophil | Protease | Modifies the functions of NK cells, monocytes |
| | | | and granulocytes |
| F3 | F3 | enzyme/redox | AKA thromboplastin, Coagulation Factor 3; |
| | | | cell surface glycoprotein responsible for |
| | | | coagulation catalysis |
| FCGR1A | Fc fragment of IgG, | Membrane | Membrane receptor for CD64; found in |
| | high affinity receptor | protein | monocytes, macrophages and neutrophils |
| | IA | | |
| FTL | Ferritin, light | iron chelator | Intracellular, iron storage protein |
| | polypeptide | | |
| GZMB | Granzyme B | proteinase | AKA CTLA1; Necessary for target cell lysis in |
| | | | cell-mediated immune responses. Crucial for |
| | | | the rapid induction of target cell apoptosis by |
| | | | cytotoxic T cells. Inhibition of the GZMB- |
| | | | IGF2R (receptor for GZMB) interaction |
| | | | prevented GZMB cell surface binding, uptake, |
| | | | and the induction of apoptosis. |
| HLA-DRA | Major | Membrane | Anchored heterodimeric molecule; cell-surface |
| | Histocompatability | protein | antigen presenting complex |
| | Complex; class II, | | |
| | DR alpha | | |
| HMOX1 | Heme oxygenase | Enzyme/ | Endotoxin inducible |
| | (decycling) 1 | Redox | |
| HSPA1A | Heat shock protein 70 | Cell Signaling | heat shock protein 70 kDa; Molecular |
| | | and activation | chaperone, stabilizes AU rich mRNA |
| HIST1H1C | Histo 1, Hic | Basic nuclear | responsible for the nucleosome structure |
| | | protein | within the chromosomal fiber in |
| | | | eukaryotes; may attribute to modification of |
| | | | nitrotyrosine-containing proteins and their |
| | | | immunoreactivity to antibodies against |
| | | | nitrotyrosine. |
| ICAM1 | Intercellular adhesion | Cell Adhesion/ | Endothelial cell surface molecule; regulates cell |
| | molecule 1 | Matrix | adhesion and trafficking, unregulated during |
| | | Protein | cytokine stimulation |
| IFI16 | Gamma interferon | Cell signaling | Transcriptional repressor |
| | inducible protein 16 | and activation | |
| IFNA2 | Interferon, alpha 2 | Cytokines- | interferon produced by macrophages with |
| | | chemokines- | antiviral effects |
| | | growth factors | |
| IFNG | Interferon, Gamma | Cytokines/ | Pro- and anti-inflammatory activity; TH1 |
| | | Chemokines/ | cytokine; nonspecific inflammatory mediator; |
| | | Growth | produced by activated T-cells. |
| | | Factors | |
| IL10 | Interleukin 10 | Cytokines- | Anti-inflammatory; TH2; suppresses production |
| | | chemokines- | of proinflammatory cytokines |
| | | growth factors | |
| IL12B | Interleukin 12 p40 | Cytokines- | Proinflammatory; mediator of innate immunity, |
| | | chemokines- | TH1 cytokine, requires co-stimulation with IL- |
| | | growth factors | 18 to induce IFN-g |
| IL13 | Interleukin 13 | Cytokines/ | Inhibits inflammatory cytokine production |
| | | Chemokines/ | |
| | | Growth | |
| | | Factors | |
| IL18 | Interleukin 18 | Cytokines- | Proinflammatory, TH1, innate and acquired |
| | | chemokines- | immunity, promotes apoptosis, requires co- |
| | | growth factors | stimulation with IL-1 or IL-2 to induce TH1 |
| | | | cytokines in T- and NK-cells |
| IL18R1 | Interleukin 18 | Membrane | Receptor for interleukin 18; binding the agonist |
| | receptor 1 | protein | leads to activation of NFKB-B; belongs to IL1 |
| | | | family but does not bind IL1A or IL1B. |
| IL1A | Interleukin 1, alpha | Cytokines- | Proinflammatory; constitutively and inducibly |
| | | chemokines- | expressed in variety of cells. Generally |
| | | growth factors | cytosolic and released only during severe |
| | | | inflammatory disease |
| IL1B | Interleukin 1, beta | Cytokines- | Proinflammatory; constitutively and inducibly |
| | | chemokines- | expressed by many cell types, secreted |
| | | growth factors | |
| IL1R1 | Interleukin 1 | Cell signaling | AKA: CD12 or IL1R1RA; Binds all three |
| | receptor, type I | and activation | forms of interleukin-1 (IL1A, IL1B and |
| | | | IL1RA). Binding of agonist leads to NFKB |
| | | | activation |
| IL1RN | Interleukin 1 | Cytokines/ | IL1 receptor antagonist; Anti-inflammatory; |
| | Receptor Antagonist | Chemokines/ | inhibits binding of IL-1 to IL-1 receptor by |
| | | Growth | binding to receptor without stimulating IL-1- |
| | | Factors | like activity |
| IL2 | Interleukin 2 | Cytokines/ | T-cell growth factor, expressed by activated T- |
| | | Chemokines/ | cells, regulates lymphocyte activation and |
| | | Growth | differentiation; inhibits apoptosis, TH1 cytokine |
| | | Factors | |
| IL4 | Interleukin 4 | Cytokines/ | Anti-inflammatory; TH2; suppresses |
| | | Chemokines/ | proinflammatory cytokines, increases |
| | | Growth | expression of IL-1RN, regulates lymphocyte |
| | | Factors | activation |
| IL5 | Interleukin 5 | Cytokines/ | Eosinophil stimulatory factor; stimulates late B |
| | | Chemokines/ | cell differentiation to secretion of Ig |
| | | Growth | |
| | | Factors | |
| IL6 | Interleukin 6 | Cytokines- | Pro- and anti-inflammatory activity, TH2 |
| | (interferon, beta 2) | chemokines- | cytokine, regulates hematopoietic system and |
| | | growth factors | activation of innate response |
| IL8 | Interleukin 8 | Cytokines- | Proinflammatory, major secondary |
| | | chemokines- | inflammatory mediator, cell adhesion, signal |
| | | growth factor | transduction, cell-cell signaling, angiogenesis, |
| | | | synthesized by a wide variety of cell types |
| IL15 | Interleukin 15 | cytokines- | Proinflammatory, mediates T-cell activation, |
| | | chemokines- | inhibits apoptosis, synergizes with IL-2 to |
| | | growth factors | induce IFN-g and TNF-a |
| IRF5 | interferon regulatory | Transcription | possess a novel helix-turn-helix DNA-binding |
| | factor 5 | factor | motif and mediate virus- and interferon (IFN)- |
| | | | induced signaling pathways. |
| IRF7 | Interferon regulatory | Transcription | Regulates transcription of interferon genes |
| | factor 7 | Factor | through DNA sequence-specific binding. |
| | | | Diverse roles include virus-mediated activation |
| | | | of interferon, and modulation of cell growth, |
| | | | differentiation, apoptosis, and immune system |
| | | | activity. |
| ITGA-4 | integrin alpha 4 | integrin | receptor for fibronectin and VCAM1; triggers |
| | | | homotypic aggregation for VLA4 positive |
| | | | leukocytes; participates in cytolytic T-cell |
| | | | interactions with target cells. |
| ITGAM | Integrin, alpha M; | integrin | AKA: Complement receptor, type 3, alpha |
| | complement receptor | | subunit; neutrophil adherence receptor; role in |
| | | | adherence of neutrophils and monocytes to |
| | | | activate endothelium |
| LBP | Lipopolysaccharide | membrane | Acute phase protein; membrane protein that |
| | binding protein | protein | binds to Lipid a moity of bacterial LPS |
| LTA | LTA (lymphotoxin | Cytokine | Cytokine secreted by lymphocytes and |
| | alpha) | | cytotoxic for a range of tumor cells; active in |
| | | | vitro and in vivo |
| LTB | Lymphotoxin beta | Cytokine | Inducer of inflammatory response and normal |
| | (TNFSF3) | | lymphoid tissue development |
| JUN | v-jun avian sarcoma | Transcription | Proto-oncoprotein; component of transcription |
| | virus 17 oncogene | factor-DNA | factor AP-1 that interacts directly with target |
| | homolog | binding | DNA sequences to regulate gene expression |
| MBL2 | Mannose-binding | lectin | AKA: MBP1; mannose binding protein C |
| | protein | | precursor |
| MIF | Macrophage | Cell signaling | AKA; GIF; lymphokine, regulators macrophage |
| | migration inhibitory | and growth | functions through suppression of anti- |
| | factor | factor | inflammatory effects of glucocorticoids |
| MMP9 | Matrix | proteinase | AKA gelatinase B; degrades extracellular |
| | metalloproteinase 9 | | matrix molecules, secreted by IL-8-stimulated |
| | | | neutrophils |
| MMP3 | Matrix | proteinase | capable of degrading proteoglycan, fibronectin, |
| | metalloproteinase 3 | | laminin, and type IV collagen, but not |
| | | | interstitial type I collagen. |
| MX1 | Myxovirus resistance | peptide | Cytoplasmic protein induced by influenza; |
| | 1; interferon | | associated with MS |
| | inducible protein p78 | | |
| N33 | Putative prostate | Tumor | Integral membrane protein. Associated with |
| | cancer tumor | Suppressor | homozygous deletion in metastatic prostate |
| | suppressor | | cancer. |
| NFKB1 | Nuclear factor of | Transcription | p105 is the precursor of the p50 subunit of the |
| | kappa light | Factor | nuclear factor NFKB, which binds to the kappa- |
| | polypeptide gene | | b consensus sequence located in the enhancer |
| | enhancer in B-cells 1 | | region of genes involved in immune response |
| | (p105) | | and acute phase reactions; the precursor does |
| | | | not bind DNA itself |
| NFKBIB | Nuclear factor of | Transcription | Inhibits/regulates NFKB complex activity by |
| | kappa light | Regulator | trapping NFKB in the cytoplasm. |
| | polypeptide gene | | Phosphorylated serine residues mark the |
| | enhancer in B-cells | | NFKBIB protein for destruction thereby |
| | inhibitor, beta | | allowing activation of the NFKB complex. |
| NOS1 | nitric oxide synthase | enzyme/redox | synthesizes nitric oxide from L-arginine and |
| | 1 (neuronal) | | molecular oxygen, regulates skeletal muscle |
| | | | vasoconstriction, body fluid homeostasis, |
| | | | neuroendocrine physiology, smooth muscle |
| | | | motility, and sexual function |
| NOS3 | Nitric oxide synthase 3 | enzyme/redox | enyzme found in endothelial cells mediating |
| | | | smooth muscle relation; promotes clotting |
| | | | through the activation of platelets |
| PAFAH1B1 | Platelet activating | Enyzme | Inactivates platelet activating factor by |
| | factor | | removing the acetyl group |
| | acetylhydrolase, | | |
| | isoform !b, alpha | | |
| | subunit; 45 kDa | | |
| PF4 | Platelet Factor 4 | Chemokine | PF4 is released during platelet aggregation and |
| | (SCYB4) | | is chemotactic for neutrophils and monocytes. |
| | | | PF4's major physiologic role appears to be |
| | | | neutralization of heparin-like molecules on the |
| | | | endothelial surface of blood vessels, thereby |
| | | | inhibiting local antithrombin III activity and |
| | | | promoting coagulation. |
| PI3 | Proteinase inhibitor 3 | Proteinase | aka SKALP; Proteinase inhibitor found in |
| | skin derived | inhibitor- | epidermis of several inflammatory skin |
| | | protein | diseases; it's expression can be used as a marker |
| | | binding- | of skin irritancy |
| | | extracellular | |
| | | matrix | |
| PLA2G7 | Phospholipase A2, | Enzyme/ | Platelet activating factor |
| | group VII (platelet | Redox | |
| | activating factor | | |
| | acetylhydrolase, | | |
| | plasma) | | |
| PLAU | Plasminogen | proteinase | AKA uPA; cleaves plasminogen to plasmin (a |
| | activator, urokinase | | protease responsible for nonspecific |
| | | | extracellular matrix degradation; UPA |
| | | | stimulates cell migration via a UPA |
| | | | receptor |
| PLAUR | plasminogen | Membrane | key molecule in the regulation of cell- |
| | activator, urokinase | protein; | surface plasminogen activation; also |
| | receptor | receptor | involved in cell signaling. |
| PTGS2 | Prostaglandin- | Enzyme | Key enzyme in prostaglandin biosynthesis and |
| | endoperoxide | | induction of inflammation |
| | synthase 2 | | |
| PTX3 | Pentaxin-related | Acute Phase | AKA TSG-14; Pentaxin 3; Similar to the |
| | gene, rapidly induced | Protein | pentaxin subclass of inflammatory acute-phase |
| | by IL-1 beta | | proteins; novel marker of inflammatory |
| | | | reactions |
| RAD52 | RAD52 (S. cerevisiae) | DNA binding | Involved in DNA double-stranded break repair |
| | homolog | proteinsor | and meiotic/mitotic recombination |
| SERPINE1 | Serine (or cysteine) | Proteinase/ | Plasminogen activator inhibitor-1/PAI-1 |
| | protease inhibitor, | Proteinase | |
| | clade B (ovalbumin), | Inhibitor | |
| | member 1 | | |
| SFTPD | Surfactant, | extracellular | AKA: PSPD; mannose-binding protein; |
| | pulomonary | lipoprotein | suggested role in innate immunity and |
| | associated protein D | | surfactant metabolism |
| SLC7A1 | Solute carrier family | Membrane | High affinity, low capacity permease invovled |
| | 7, member 1 | protein; | in the transport of positively charged amino |
| | | permease | acids |
| SPP1 | secreted | cell signaling | binds vitronectin; protein ligand of CD44, |
| | phosphoprotein 1 | and activation | cytokine for type 1 responses mediated by |
| | (osteopontin) | | macrophages |
| STAT3 | Signal transduction | Transcription | AKA APRF: Transcription factor for acute |
| | and activator of | factor | phase response genes; rapidly activated in |
| | transcription 3 | | response to certain cytokines and growth |
| | | | factors; binds to IL6 response elements |
| TGFBR2 | Transforming growth | Membrane | AKA: TGFR2; membrane protein involved in |
| | factor, beta receptor | protein | cell signaling and activation, ser/thr protease; |
| | II | | binds to DAXX. |
| TIMP1 | Tissue inhibitor of | Proteinase/ | Irreversibly binds and inhibits |
| | metalloproteinase 1 | Proteinase | metalloproteinases, such as collagenase |
| | | Inhibitor | |
| TLR2 | toll-like receptor 2 | cell signaling | mediator of petidoglycan and lipotechoic acid |
| | | and activation | induced signaling |
| TLR4 | Toll-like receptor 4 | Cell signaling | mediator of LPS induced signaling |
| | | and activation | |
| TNF | Tumor necrosis factor | Cytokine/tumor | Negative regulation of insulin action. Produced |
| | | necrosis | in excess by adipose tissue of obese individuals - |
| | | factor receptor | increases IRS-1 phosphorylation and |
| | | ligand | decreases insulin receptor kinase activity. Pro- |
| | | | inflammatory; TH₁ cytokine; Mediates host |
| | | | response to bacterial stimulus; Regulates cell |
| | | | growth & differentiation |
| TNFRSF7 | Tumor necrosis factor | Membrane | Receptor for CD27L; may play a role in |
| | receptor superfamily, | protein; | activation of T cells |
| | member 7 | receptor | |
| TNFSF13B | Tumor necrosis factor | Cytokines- | B cell activating factor, TNF family |
| | (ligand) superfamily, | chemokines- | |
| | member 13b | growth factors | |
| TNFRSF13B | Tumor necrosis factor | Cytokines- | B cell activating factor, TNF family |
| | receptor superfamily, | chemokines- | |
| | member 13, subunit | growth factors | |
| | beta | | |
| TNFSF5 | Tumor necrosis factor | Cytokines- | Ligand for CD40; expressed on the surface of T |
| | (ligand) superfamily, | chemokines- | cells. It regulates B cell function by engaging |
| | member 5 | growth factors | CD40 on the B cell surface. |
| TNFSF6 | Tumor necrosis factor | Cytokines- | AKA FasL; Ligand for FAS antigen; transduces |
| | (ligand) superfamily, | chemokines- | apoptotic signals into cells |
| | member 6 | growth factors | |
| TREM1 | Triggering receptor | cell signaling | Member of the Ig superfamily; receptor |
| | expressed on myeloid | and activation | exclusively expressed on myeloid cells. |
| | cells 1 | | TREM1 mediates activation of neutrophils and |
| | | | monocytes and may have a predominant role in |
| | | | inflammatory responses |
| VEGF | vascular endothelial | cytokines- | VPF; Induces vascular permeability, endothelial |
| | growth factor | chemokines- | cell proliferation, angiogenesis. Producted by |
| | | growth factors | monocytes |

1. A method for determining a profile data set for a subject with multiple sclerosis or inflammatory conditions related to multiple sclerosis based on a sample from the subject, the sample providing a source of RNAs, the method comprising:
using amplification for measuring the amount of RNA corresponding to at least 2 constituents from Table 1 and
arriving at a measure of each constituent,
wherein the profile data set comprises the measure of each constituent and wherein amplification is performed under measurement conditions that are substantially repeatable.

2. A method according to claim 1, wherein the subject has presumptive signs of a multiple sclerosis including at least one of: altered sensory, motor, visual or proprioceptive system with at least one of numbness or weakness in one or more limbs, often occurring on one side of the body at a time or the lower half of the body, partial or complete loss of vision, frequently in one eye at a time and often with pain during eye movement, double vision or blurring of vision, tingling or pain in numb areas of the body, electric-shock sensations that occur with certain head movements, tremor, lack of coordination or unsteady gait, fatigue, dizziness, muscle stiffness or spasticity, slurred speech, paralysis, problems with bladder, bowel or sexual function, and mental changes such as forgetfulness or difficulties with concentration, relative to medical standards.

3. (canceled)

4. A method for determining a profile data set according to claim 1, wherein the measurement conditions that are substantially repeatable are within a degree of repeatability of better than five percent.

5. (canceled)

6. A method for determining a profile data set according to claim 1, wherein efficiencies of amplification for all constituents are substantially similar.

7-9. (canceled)

10. A method of characterizing multiple sclerosis or inflammatory conditions related to multiple sclerosis in a subject, based on a sample from the subject, the sample providing a source of RNAs, the method comprising:
assessing a profile data set of a plurality of members, each member being a quantitative measure of the amount of a distinct RNA constituent in a panel of constituents selected so that measurement of the constituents enables characterization of the presumptive signs of a multiple sclerosis, wherein such measure for each constituent is obtained under measurement conditions that are substantially repeatable.

11. A method according to claim 10, wherein the subject has presumptive signs of a multiple sclerosis including at least one of: altered sensory, motor, visual or proprioceptive system with at least one of numbness or weakness in one or more limbs, often occurring on one side of the body at a time or the lower half of the body, partial or complete loss of vision, frequently in one eye at a time and often with pain during eye movement, double vision or blurring of vision, tingling or pain in numb areas of the body, electric-shock sensations that occur with certain head movements, tremor, lack of coordination or unsteady gait, fatigue, dizziness, muscle stiffness or spasticity, slurred speech, paralysis, problems with bladder, bowel or sexual function, and mental changes such as forgetfulness or difficulties with concentration, relative to medical standards.

12. A method for characterizing multiple sclerosis or inflammatory conditions related to multiple sclerosis in a subject according to claim 10, wherein assessing further comprises:
comparing the profile data set to a baseline profile data set for the panel, wherein the baseline profile data set is related to the multiple sclerosis or inflammatory conditions related to multiple sclerosis to be characterized.

13. (cancelled)

14. A method according to claim 10, wherein the multiple sclerosis or inflammatory conditions related to multiple sclerosis are with respect to a localized tissue of the subject and the sample is derived from a tissue of fluid of a type distinct from that of the localized tissue.

15-16. (canceled)

17. A method for evaluating multiple sclerosis or inflammatory conditions related to multiple sclerosis in a subject based on a first sample from the subject, the sample providing a source of RNAs, the method comprising:
deriving from the first sample a first profile data set, the profile data set including a plurality of members, each member being a quantitative measure of the amount of a distinct RNA constituent in a panel of constituents selected so that measurement of the constituents enables evaluation of the multiple sclerosis or inflammatory conditions related to multiple sclerosis wherein such measure for each constituent is obtained under measurement conditions that are substantially repeatable; and
producing a calibrated profile data set for the panel, wherein each member of the calibrated profile data set is a function of a corresponding member of the first profile data set and a corresponding member of a baseline profile data set for the panel, and wherein the baseline profile data set is related to the multiple sclerosis or inflammatory conditions related to multiple sclerosis to be evaluated,
the calibrated profile data set being a comparison between the first profile data set and the baseline profile data set, thereby providing evaluation of the multiple sclerosis or inflammatory conditions related to multiple sclerosis of the subject.

18. A method according to claim 17, wherein the subject has presumptive signs of a multiple sclerosis including at least one of: altered sensory, motor, visual or proprioceptive system with at least one of numbness or weakness in one or more limbs, often occurring on one side of the body at a time or the lower half of the body, partial or complete loss of vision, frequently in one eye at a time and often with pain during eye movement, double vision or blurring of vision, tingling or pain in numb areas of the body, electric-shock sensations that occur with certain head movements, tremor, lack of coordination or unsteady gait, fatigue, dizziness, muscle stiffness or spasticity, slurred speech, paralysis, problems with bladder, bowel or sexual function, and mental changes such as forgetfulness or difficulties with concentration, relative to medical standards.

19. A method according to claim 17, wherein the baseline profile data set is derived from one or more other samples from the same subject taken under circumstances different from those of the first sample.

20. A method according to claim 19, wherein the circumstances are selected from the group consisting of (i) the time at which the first sample is taken, (ii) the site from which the first sample is taken, (iii) the biological condition of the subject when the first sample is taken.

21-24. (canceled)

25. A method according to claim 17, wherein the first sample is derived from blood and the baseline profile data set is derived from tissue or body fluid of the subject other than blood.

26. A method according to claim 17, wherein the first sample is derived from tissue or body fluid of the subject and the baseline profile data set is derived from blood.

27. A method according to claim 19, wherein the baseline profile data set is derived from one or more other samples from the same subject, taken when the subject is in a biological condition different from that in which the subject was at the time the first sample was taken, with respect to at least one of age, nutritional history, medical condition, clinical indicator, medication, physical activity, body mass, and environmental exposure.

28. A method according to claim 17, wherein the baseline profile data set is derived from one or more other samples from one or more different subjects.

29. A method according to claim 28, wherein the one or more different subjects have in common with the subject at least one of age group, gender, ethnicity, geographic location, nutritional history, medical condition, clinical indicator, medication, physical activity, body mass, and environmental exposure.

30. A method according to claim 29, wherein a clinical indicator has been used to assess multiple sclerosis or inflammatory conditions related to multiple sclerosis of the one or more different subjects, further comprising: interpreting the calibrated profile data set in the context of at least one other clinical indicator.

31. A method according to claim 30, wherein the at least one other clinical indicator is selected from the group consisting of blood chemistry, urinalysis, X-ray or other radiological or metabolic imaging technique, other chemical assays, and physical findings.

32-38. (canceled)

39. A method according to claim 17, wherein the quantitative measure is determined by amplification, and the measurement conditions are such that efficiencies of amplification for all constituents differ by less than approximately 2 percent.

40-179. (canceled)
